(19) United States
(12) Patent Application Publication
(10) Pub. No.: US 2005/0118286 A1
(43) Pub. Date: Jun. 2, 2005

(54) Compositions and methods for treatment of nervous system disorders

(75) Inventors: Stephen C. Suffin, Sherman Oaks, CA (US); W. Hamlin Emory, Malibu, CA (US); Leonard Brandt, San Juan Capistrano, CA (US)

Correspondence Address: MEDLEN & CARROLL, LLP;Suite 350, 101 Howard Street, San Francisco, CA, 94105, US

(73) Assignee: CNS Response

(21) Appl. No.: 10/972,188

(22) Filed: Oct. 22, 2004

Related U.S. Application Data
(63) Continuation of application No. 10/697,497, filed on Oct. 30, 2003.

(51) Int. Cl. A61K 38/05; A61K 31/5377; A61K 31/522; A61K 31/445; A61K 31/137; A61K 31/138; A61K 31/403; A61K 35/78

(52) U.S. Cl. 424/752; 514/18; 514/263.31; 514/411; 514/317; 514/649; 514/651; 514/618; 514/235.5

(57) ABSTRACT

The present invention contemplates compositions and methods to treat patients having a nervous system disorder with a formulation comprising an anticonvulsant and a neuroactive modulator. Also described is a method to predict the probability of a significant recovery when a treating an individual patient having a nervous system disorder with a formulation comprising an anticonvulsant and a neuroactive modulator. Specifically, methods for predicting patient prognosis include, but are not limited to, quantitative electroencephalography, psychometric test batteries, biological indicators, brain metabolic indicators, genotype profiles, neuroimaging, objective test measurements and multi-modalities. The present invention also discloses a device providing an organized dispensation of the above formulations such that the patient or medical personnel may easily and accurately decrease the daily dosage of a third drug and increase the daily dosage of a formulation comprising an anticonvulsant and a neuroactive modulator.

### FIELD OF INVENTION

In one embodiment, this invention relates to predicting the probability of a significant recovery following pharmaceutical treatment of nervous system disorders. In one embodiment, this invention relates to predicting the probability of a significant recovery from a nervous system disorder by a combination of at least two pharmaceutical formulations. In another embodiment, this invention relates to predicting the probability of a significant recovery following the treatment of nervous system disorders by at least one pharmaceutical formulation combined with a medical device. In another embodiment, this invention relates to predicting the probability of a significant recovery following the treatment of nervous system disorders by a combination of an anticonvulsant and a neuroactive modulator.

### BACKGROUND

Nervous system disorders are known to encompass a wide variety of clinically significant conditions. In general, primary psychiatric disorders are categorized according to the Diagnostic and Statistical Manual of Mental Disorders, 4th Edition (i.e., referred to hereinafter as DSM-IV) and may be represented as: i) Disorders Usually First Diagnosed in Infancy, Childhood, or Adolescence; ii) Cognitive Disorders; Mental Disorders Due to a General Medical Condition; iii) Substance-Related Disorders; iv) Schizophrenia and Other Psychotic Disorders; v) Mood Disorders; vi) Anxiety Disorders; vii) Somatoform Disorders; Factitious Disorder; Dissociative Disorders; viii) Sexual and Gender Identity Disorders; ix) Eating Disorders; Sleep Disorders; x) Impulse-Control Disorders Not Elsewhere Classified; Adjustment Disorder; and xi) Personality Disorders. Neurologically based diseases, however, are also properly defined in terms of a nervous system disorder. Current clinical treatment for both psychiatric disorders and neurological diseases is generally pharmaceutically-oriented. However, in psychiatric disorders an emphasis is also placed upon a critical patient psychotherapy.

The use of prescription drugs for psychiatric disorders is generally recognized, for example: i) neuroleptic or antipsychotic drugs for severe psychotic illness, ii) mood-stabilizing or antidepressant drugs to generally treat affective disorders, and iii) antianxiety or sedative drugs to treat anxiety states or other related conditions and vi) stimulants to treat hyperactive or attention deficit disorders. Successful long-term treatment, however, is problematic due to physiological adaptations involving tolerance, addiction and refractoriness. In addition to these shortcomings, problems involving less-than-dramatic efficacy is not unusual. Baldessarini R. J., “Drugs and Treatment Of Psychiatric Disorders” In: Goodman and Gilman's The Pharmacological Basis Of Therapeutics, Eighth Edition, Goodman et al., Eds, Permagon Press, New York (1990).

Traditionally, nervous system disorders have been treated by sequentially administering a single drug (i.e., monotherapy) where partially effective monotherapeutic drugs are combined until a fully effective combination is found (i.e., a trial and error method, inherently incorporating a large degree of random chance). For example, the most commonly used drugs for depressive disorders include the tricyclic antidepressants, selective serotonin reuptake inhibitors, selective norepinephrine reuptake inhibitors, lithium carbonate, and the monoamine oxidase inhibitors. These drugs are suggested to increase the synaptic level of neurotransmitters, most notably, norepinephrine, serotonin and dopamine. Though some success has been achieved in the treatment of depression using a monotherapy approach, however, a significant number of patients are either non-responsive (i.e., refractory) or whose symptomology actually worsens following the standard course of monotherapy.

It is well acknowledged in the art that the treatment of nervous system disorders is complicated by diagnostic uncertainties. Successful efforts to maximize the association between specific clinical syndromes and predictable responses indicate that treatment of nervous system disorders may one day transition from a state of art, to one of science. Efforts to solve the problem of patient response and improved efficacy of drug therapy has become a primary focus of nervous system treatment and a reliable method to predict patient response prior to treatment is clearly need. Also, often what is clearly needed is a safe and effective pharmaceutical combination designed for long-term treatment in patients exhibiting nervous system disorders.

### SUMMARY

In one embodiment, this invention relates to predicting the probability of a significant recovery following pharmaceutical treatment of nervous system disorders. In one embodiment, this invention relates to predicting the probability of a significant recovery from a nervous system disorder by a pharmaceutical formulation. In another embodiment, this invention relates to predicting the probability of a significant recovery following the treatment of nervous system disorders by at least one pharmaceutical formulation combined with a medical device. In another embodiment, this invention relates to predicting the probability of a significant recovery following the treatment of nervous system disorders by a formulation comprising an anticonvulsant and a neuroactive modulator.

One advantage of the present invention contemplates a composition comprising a formulation comprising an anticonvulsant and a neuroactive modulator. In one embodiment, the formulation comprises oxcarbazepine and the neuroactive modulator. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion. In one embodiment, the monoaminergic reuptake inhibitor comprises a noradrenergic/dopaminergic reuptake inhibitor. In one embodiment, the noradrenergic/dopaminergic reuptake inhibitor comprises hydroxybupropion. In another embodiment, the monoaminergic reuptake inhibitor is a selective noradrenergic reuptake inhibitor. In one embodiment, the selective noradrenergic reuptake inhibitor comprises an optically pure (S,S)-hydroxybupropion. In another embodiment, the form of said formulation includes, but is not limited to, tablets, oral liquids, intrapulmonary liquids, capsules, transdermal patches, polymer-coated tablets, liposomes, microspheres, aerosols, fast-dissolve compounds or sterile injectable solutions.

One advantage of the present invention contemplates a pharmaceutical formulation comprising oxcarbazepine and an antidepressant, wherein said antidepressant is selected from the group comprising bupropion, bupropion derivatives or bupropion metabolites. In one embodiment, the pharmaceutical formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the form of the formulation is selected from the group comprising tablets, capsules, oral liquids, intrapulmonary liquids, transdermal patches, polymer-coated tablets, microparticles, nanoparticles, aerosols, fast-dissolve compounds or sterile injectable solutions.

Another advantage of the present invention contemplates a pharmaceutical formulation comprising oxcarbazepine and a neurotransmitter reuptake inhibitor, wherein said inhibitor is selected from the group comprising a dopaminergic reuptake inhibitor, a noradrenergic/serotonergic reuptake inhibitor, a glutaminergic reuptake inhibitor, a glycine reuptake inhibitor and a GABA reuptake inhibitor. In one embodiment, the pharmaceutical formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the form of the pharmaceutical formulation is selected from the group comprising tablets, capsules, oral liquids, intrapulmonary liquids, transdermal patches, polymer-coated tablets, microparticles, nanoparticles, aerosols, fast-dissolve compounds or sterile injectable solutions.

Another advantage of the present invention contemplates a pharmaceutical formulation comprising oxcarbazepine and a noradrenergic reuptake inhibitor, wherein said inhibitor is selected from the group comprising imipramine, amitryptyline, desiprimine, clomipramine, desmethyldlomipramine, nortryptyline, doxepine, protryptyline, maprotiline, nisoxetine, tomoxetine, robxetine and lofepramine. In one embodiment, the formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the form of the formulation is selected from the group comprises tablets, capsules, oral liquids, intrapulmonary liquids, transdermal patches, polymer-coated tablets, microparticles, nanoparticles, aerosols, fast-dissolve compounds or sterile injectable solutions.

Another advantage of the present invention contemplates a pharmaceutical formulation comprising oxcarbazepine and a selective serotonergic reuptake inhibitor, wherein the inhibitor is selected from the group comprising fluoxetine, sertraline, paroxetine, fluvoxamine, nefazodone, hyperforin and RO-15-808. In one embodiment, the formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the form of the formulation is selected from the group comprising tablets, capsules, oral liquids, intrapulmonary liquids, transdermal patches, polymer-coated tablets, microparticles, nanoparticles, aerosols, fast-dissolve compounds or sterile injectable solutions.

Another advantage of the present invention contemplates a pharmaceutical formulation comprising oxcarbazepine and a neuroactive modulator, wherein the neuroactive modulator is selected from the group comprising a neurotransmitter metabolic inhibitor, an acetylcholine receptor agent, a glycine receptor agent, a GABA receptor agent, an NMDA receptor agent. In one embodiment, the pharmaceutical formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the form of the formulation is selected from the group comprising tablets, capsules, oral liquids, intrapulmonary liquids, transdermal patches, polymer-coated tablets, microparticles, nanoparticles, aerosols, fast-dissolve compounds or sterile injectable solutions.

Another advantage of the present invention contemplates a method of treatment, comprising: i) providing a patient exhibiting at least one symptom of a nervous system disorder; and ii) administering to said patient a formulation comprising an anticonvulsant and a neuroactive modulator such that at least one symptom of the nervous system disorder is reduced. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the formulation comprises oxcarbazepine and the neuroactive modulator. In one embodiment, the formulation further comprises a third drug, wherein said third drug comprises selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents or nutriceuticals. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor includes, but is not limited to, a monoaminergic, glycinergic, glutaminergic or GABAeric reuptake inhibitor. In one embodiment, the monoaminergic reuptake inhibitor comprises bupropion. In another embodiment, the formulation comprises a compounded formulation. In another embodiment, the compounded formulation further comprises said third drug. In one embodiment, the anticonvulsant, the neuroactive modulator and/or the third drug are sequentially administered within forty-eight hours, preferably within twenty-four hours and more preferably within twelve hours. In another embodiment, the formulation comprises a divided daily dose ratio between the anticonvulsant and the monoaminergic reuptake inhibitor wherein said ratio ranges approximately between 4000/25-150/750 milligrams. In one embodiment, the anticonvulsant includes, but is not limited to, oxcarbazepine, 10-OH-carbazepine and carbazepine-10,11-trans-diol. In one embodiment, the monoaminergic reuptake inhibitor comprises bupropion. In another embodiment, the formulation comprises a divided daily dose ratio between oxcarbazepine and bupropion wherein said ratio includes, but not is limited to, 4000/25, 3700/75, 3400/125, 3100/175, 2800/325, 2500/375, 2200/425, 1900/475, 1600/525, 1300/575, 1000/625, 700/675, 400/725 or 150/750 milligrams. In one embodiment, the form of the formulation includes, but is not limited to, tablets, capsules, oral liquids, intrapulmonary liquids, transdermal patches, polymer-coated tablets, liposomes, microspheres, aerosols, fast-dissolve compounds and a sterile injectable solutions.

Another advantage of the present invention contemplates a method of treatment, comprising: i) providing a patient exhibiting at least one symptom of a nervous system disorder; and ii) administering to said patient a formulation comprising an anticonvulsant and a selective serotonergic reuptake inhibitor such that at least one of said symptoms of said nervous system disorder is reduced. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the anticonvulsant comprises oxcarbazepine. In another embodiment, the formulation comprises a compounded formulation. In one embodiment, the anticonvulsant and the selective serotonin reuptake inhibitor are sequentially administered within forty-eight hours, preferably within twenty-four hours and more preferably within twelve hours. In one embodiment, the form of the formulation includes, but is not limited to, tablets, capsules, oral liquids, intrapulmonary liquids, transdermal patches, polymer-coated tablets, liposomes, microspheres, aerosols, fast-dissolve compounds and sterile injectable solutions. In one embodiment, the formulation comprises a divided daily dose ratio between the oxcarbazepine and the selective serotonergic reuptake inhibitor wherein said ratio ranges from approximately 4000/5-150/250 milligrams. In another embodiment, the formulation comprises a divided daily dose ratio between the oxcarbazepine and the selective serotonergic reuptake inhibitor wherein said ratio includes, but is not limited to, 4000/25, 3700/40, 3400/55, 3100/70, 2800/85, 2500/100, 2200/115, 1900/130, 1600/145, 1300/160, 1000/175, 700/190, 400/225 or 150/250 milligrams.

Another advantage of the present invention contemplates a method of treatment, comprising: i) providing a patient exhibiting at least one symptom of a nervous system disorder; ii) administering said patient with a formulation comprising an anticonvulsant; and, iii) treating said patient with a neurostimulation device such that at least one of said symptoms of said nervous system disorder is reduced. In one embodiment, the anticonvulsant comprises oxcarbazepine. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the electrostimulation device includes, but is not limited to, subepidermal implantation, nerve implantation (i.e., for example, a peripheral nervous system nerve, a central nervous system nerve) or electroconvulsant therapy. In one embodiment, the form of the anticonvulsant formulation includes, but is not limited to, tablets, capsules, oral liquids, intrapulmonary liquids, transdermal patches, polymer-coated tablets, liposomes, microspheres, aerosols, fast-dissolve compounds and sterile injectable solutions. In one embodiment, the formulation comprises a divided daily dose of oxcarbazepine ranging from approximately 4000-250 milligrams, preferably, from approximately, 3000-1000 mgs, more preferably from approximately 2500-1500 milligrams.

Another advantage of the present invention contemplates a method of treatment, comprising: i) providing a patient exhibiting at least one symptom of a nervous system disorder and is being treated with a dose of a third drug, wherein said patient is non-remissive; ii) administering to said patient a formulation comprising a divided daily dose of an anticonvulsant and a divided daily dose of a neuroactive modulator such that at least one symptom of the nervous system disorder is reduced. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the formulation further comprises said third drug. In one embodiment, the anticonvulsant, the neuroactive modulator and/or the third drug are sequentially administered within forty-eight hours, preferably within twenty-four hours and more preferably within twelve hours. In one embodiment, the method further comprises step (c) decreasing the daily divided dose of the third drug. In another embodiment, the administering of step (b) is performed over a period of time such that the daily divided dose of the oxcarbazepine and the bupropion is increased. In one embodiment, the nervous system disorder comprises depression. In one embodiment, the third drug is selected from the group comprising selective serotonin reuptake inhibitors, monoamine oxidase inhibitors, antipsychotic drugs, antianxiety/anxiolytic drugs, barbituates, stimulants, antiparkinsonian drugs, analgestic drugs, cardiac agents or nutriceuticals. In one embodiment, the non-remissive patient is refractory to the third drug. In another embodiment, the non-remissive patient has an insignificant response to the third drug. In one embodiment, the non-remissive patient is identified by neuroelectrophysiological measurements, including, but not limited to, power, coherence, symmetry, frequency and relative power. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion. In one embodiment, the formulation is administered as a compounded formulation. In another embodiment, the compounded formulation further comprises the third drug. In one embodiment, the form of the formulation or compounded formulation includes, but is not limited to, tablets, capsules, oral liquids, intrapulmonary liquids, transdermal patches, polymer-coated tablets, liposomes, microspheres, aerosols, fast-dissolve compounds and sterile injectable solutions.

Another advantage of the present invention contemplates a method of treatment, comprising: i) providing a patient exhibiting at least one symptom of a nervous system disorder; and ii) administering to said patient a formulation comprising an anticonvulsant and fluoxetine such that at least one of said symptoms of said nervous system disorder is reduced. In one embodiment, the anticonvulsant comprises oxcarbazepine. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In another embodiment, the fluoxetine is administered in a low dose regimen (i.e., for example, comprising doses lower than current Physician's Desk Reference recommendations and those appearing in future editions). In one embodiment, the low dose regimen comprises a divided daily dose of approximately between 10 mg-30 mg that is converted into a weekly dose of approximately 10 mg-30 mg. In one embodiment, the weekly dose is given in equal divided daily doses. In another embodiment, the weekly dose is given in a single dose. In one embodiment, the anticonvulsant and fluoxetine are sequentially administered within forty-eight hours, preferably within twenty-four hours and more preferably within twelve hours. In one embodiment, the formulation comprises a compounded formulation. In one embodiment, the form of the formulation includes, but is not limited to, tablets, capsules, oral liquids, intrapulmonary liquids, transdermal patches, polymer-coated tablets, liposomes, microspheres, aerosols, fast-dissolve compounds and sterile injectable solutions. In one embodiment, the formulation comprises a divided daily dose ratio between fluoxetine and oxcarbazepine wherein said ratio ranges between approximately 5/2500-100/500 milligrams. In another embodiment, the formulation comprises a divided daily dose ratio between fluoxetine and oxcarbazepine wherein said ratio is selected from the group comprising 5/2500, 10/2400, 20/2200, 30/2000, 40/1750, 50/1500, 60/1000, 70/750, 80/600, and 100/550 milligrams.

Another advantage of the present invention contemplates a method of treatment, comprising: i) providing a patient exhibiting at least one symptom of a nervous system disorder; and ii) administering to said patient a formulation comprising venlafaxine and a nutriceutical such that at least one of said symptoms of said nervous system disorder is reduced. In one embodiment, the nutriceutical includes, but is not limited to, Tryptophan-Phenylalanine-Glutamine, ginko biloba, essential fatty acid omega 3, essential fatty acid omega 6 and essential fatty acid omega 9.

Another advantage of the present invention contemplates a method of treatment, comprising: i) providing a patient exhibiting at least one symptom of a nervous system disorder; and ii) administering to said patient a formulation comprising venlafaxine and a stimulant compound such that at least one of said symptoms of said nervous system disorder is reduced.

Another advantage of the present invention contemplates a method of treatment, comprising: i) providing a patient exhibiting at least one symptom of a nervous system disorder; and ii) administering to said patient a formulation comprising a cardiac agent and a stimulant such that at least one of said symptoms of said nervous system disorder is reduced. In one embodiment, the stimulant includes, but is not limited to, amphetamine, dextroamphetamine, methamphetamine, modafinil (Provigil), methylphenidate, atomoxetine, ephedrine, caffeine, theophylline, theobromine, Tryptophan-Phenylalanine-Glutamine and ginko biloba.

Another advantage of the present invention contemplates a method of treatment, comprising: i) providing a patient exhibiting at least one symptom of a nervous system disorder; and ii) administering to said patient a formulation comprising a cardiac agent and a monoamine oxidase inhibitor such that at least one of said symptoms of said nervous system disorder is reduced. In one embodiment, the monoamine oxidase inhibitor comprises selegiline and meclobomide.

Yet another advantage of the present invention contemplates a method, comprising: i) providing; a) a convalescent population database comprising a first plurality of neuroelectrical scores and a patient outcome measure; b) a normative population database comprising a second plurality of neuroelectrical scores; and c) a clinical database comprising a third plurality of neuroelectrical scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category for a drug formulation, wherein the probability response category is selected from the group comprising sensitive, intermediate and resistive. In one embodiment, the patient outcome measure comprises a CGI score. In one embodiment, the method further comprises treating the patient when classified within the probability response category selected from the group comprising sensitive and intermediate with a formulation comprising an anticonvulsant and a neuroactive modulator, such that at least one symptom of the nervous system disorder is reduced. In one embodiment, the neuroelectrical score comprises data collected during tests including, but not limited to, electroencephalographic, electrophysiologic, magnetic resonance, positron emission or neurologic examinations. In one embodiment, the neuroelectrical score includes, but is not limited to, multivariate Z scores, univariate Z scores (i.e., standard deviations), probability scores and raw data. In one embodiment, the nervous system disorder may be diagnosed by measurements including, but is not limited to, electroencephalographic, electrophysiological, neurological, biochemical or behavioral or intrapulmonary. In one embodiment, the diagnosed nervous system disorder includes, but is not limited to, at least one neurobehavioral or intrapulmonary, neuropsychological, neurophysiological, or behavioral or intrapulmonary symptom. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoamine reuptake inhibitor. In one embodiment, the monoamine reuptake inhibitor comprises bupropion.

Still yet another advantage of the present invention contemplates a method, comprising: i) providing; a) a convalescent population database comprising a first plurality of neuroelectrical scores and a patient outcome measure; b) a normative population database comprising a second plurality of neuroelectrical scores; and c) a clinical database comprising a third plurality of neuroelectrical scores derived from an individual non-remissive patient to administration of a third drug, wherein the non-remissive patient is exhibiting at least one symptom of a nervous system disorder; ii) comparing the individual non-remissive patient scores with the normative database such that an abberant individual non-remissive patient score is identified; and iii) comparing the abberant individual non-remissive patient score with the convalescent database such that the non-remissive patient is classified within a probability response category for a drug formulation, wherein the probability response category is selected from the group comprising sensitive, intermediate and resistive. In one embodiment, the patient outcome measure comprises a CGI score. In one embodiment, the method further comprises treating the non-remissive patient that is classified within the prognosis category selected from the group comprising sensitive and intermediate with a pharmaceutical formulation comprising an anticonvulsant and a neuroactive modulator, wherein at least one symptom of the nervous system disorder is reduced. In one embodiment, the nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders. In one embodiment, the non-remissive patient is refractory to the third drug. In another embodiment, the non-remissive patient has an insignificant response to the third drug. In one embodiment, the non-remissive patient is identified by neuroelectrophysiological measurements, including, but not limited to, power, frequency, coherence, symmetry and relative power. In one embodiment, the neuroelectrical score comprises data collected during tests including, but not limited to, electroencephalographic, electrophysiologic, magnetic resonance, positron emission or neurologic examinations. In one embodiment, the neuroelectrical score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the neuroactive modulator includes, but is not limited to, a neurotransmitter reuptake inhibitor, a neurotransmitter receptor agent or a neurotransmitter metabolic inhibitor. In one embodiment, the anticonvulsant comprises oxcarbazepine. In one embodiment, the neurotransmitter reuptake inhibitor comprises a monoaminergic reuptake inhibitor. In one embodiment, the monoaminergic reuptake inhibitor comprises bupropion. In one embodiment, the third drug includes, but is not limited to, selective serotonergic reuptake inhibitors, antipsychotics, antianxiety agents, barbiturates, antiparkinsonians, analgesics, cardiac drugs, stimulants, monoamine oxidase inhibitors or nutraceuticals. In one embodiment, the pharmaceutical formulation is administered as a compounded formulation. In another embodiment, the compounded formulation further comprises the third drug. In one embodiment, the formulation comprising an anticonvulsant and a neuroactive modulator is administered sequentially within forty-eight hours, more preferably within twenty-four hours and most preferably within twelve hours. In one embodiment, the form of the formulation and/or compounded formulation includes, but are not limited to, a tablet, capsule, oral liquid, intrapulmonary liquid, transdermal patch, polymer-coated tablet, liposomes, microspheres, aerosol, fast-dissolve compounds and sterile injectable solution.

A further advantage of the present invention contemplates a method, comprising: i) providing; a) a convalescent population database comprising a first plurality of psychometric test battery scores and a patient outcome measure; b) a normative population database comprising a second plurality of pyschometric test battery scores; and c) a clinical database comprising a third plurality of psychometric test battery scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category for a drug formulation, wherein the probability response category is selected from the group comprising sensitive, intermediate and resistive. In one embodiment, the patient outcome measure comprises a CGI score. In one embodiment, the method further comprises treating the patient that is classified within the probability response category selected from the group comprising sensitive and intermediate with a pharmaceutical formulation comprising an anticonvulsant and a neuroactive modulator, such that at least one symptom of the nervous system disorder is reduced. In one embodiment, the psychometric test battery score comprises data collected during tests including, but not limited to, intelligence, cognitive, depression, visual interpretation or auditory examinations. In one embodiment, the psychometric test battery score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Yet still a further advantage of the present invention contemplates a method comprising: i) providing; a) a convalescent population database comprising a first plurality of biological indicator scores and a patient outcome measure; b) a normative population database comprising a second plurality of biological indicator scores; and c) a clinical database comprising a third plurality of biological indicator scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category for a drug formulation, wherein the probability response category is selected from the group comprising sensitive, intermediate and resistive. In one embodiment, the patient outcome measure comprises a CGI score. In one embodiment, the method further comprises treating the patient that is classified within the probability response category selected from the group comprising sensitive and intermediate with a pharmaceutical formulation comprising an anti-convulsant and a neuroactive modulator, wherein at least one symptom of the nervous system disorder is reduced. In one embodiment, the biological indicator score comprises data collected during tests using biological samples including, but not limited to, whole blood, serum, saliva, humoral or intrapulmonary secretions, urine, feces, tissue biopsies, proteins, hormones, fatty acids, sterols, nucleic acids, cerebrospinal fluid pressure, blood pressure, heart rate, electrolytes or minerals. In one embodiment, the biological indicator score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In another embodiment, the monoaminergic reuptake inhibitor comprises bupropion.

Another further advantage of the present invention contemplates a method, comprising: i) providing; a) a convalescent population database comprising a first plurality of regional brain cognitive indicator scores and a patient outcome measure; b) a normative population database comprising a second plurality of regional brain cognitive indicator scores; and c) a clinical database comprising a third plurality of regional brain cognitive indicator scores derived from an individual patient exhibiting at least one symptom of a nervous system disorder; ii) comparing the individual patient scores with the normative database such that an abberant individual patient score is identified; iii) comparing the abberant individual patient score to the convalescent database such that the patient is classified within a probability response category is selected from the group comprising sensitive, intermediate and resistive. In one embodiment, the patient outcome measure comprises a CGI score. In one embodiment, the method further comprises treating the patient that is classified within the probability response category selected from the group comprising sensitive and resistive with a pharmaceutical formulation comprising an anticonvulsant and a neuroactive modulator, wherein at least one symptom of the nervous system disorder is reduced. In one embodiment, the regional brain cognitive indicator score comprises data determined by methods including, but not limited to, glucose utilization, radiolabled medicine scanning, X-ray, PET, magnetic responance (i.e., for example, FMRI or NMRI), magnetoencephalography (MEEG) or SPECT. In one embodiment, the regional brain cognitive indicator score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Still yet another further advantage of the present invention contemplates a method, comprising: i) providing, a) a convalescent population database comprising a first plurality of genotype allelic profile scores and a patient outcome measure; b) a normative population database comprising a second plurality of genotype allelic profile scores; and c) a clinical database comprising a third plurality of genotype allelic profile scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category for a drug formulation, wherein the prognosis category is selected from the group comprising sensitive, intermediate and resistive. In one embodiment, the patient outcome measure comprises a CGI score. In one embodiment, the method further comprises treating the patient classified within the probability response category selected from the group comprising sensitive and intermediate with a pharmaceutical formulation comprising an anticonvulsant and a neuroactive modulator, wherein at least one symptom of the nervous system disorder is reduced. In one embodiment, the genotype allelic profile score is determined by methods including, but not limited to, phenotyping, protein electrophoresis, Western blots, amino acid sequencing, genotyping, Northern blots, nucleic acid hybridization or nucleic acid sequencing. In one embodiment, the genotype allelic profile score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Still yet another further advantage of the present invention contemplates a method, comprising: i) providing; a) a convalescent population database comprising a first plurality of anatomical neuroimaging scores and a patient outcome measure; b) a normative database comprising a second plurality of anatomical neuroimaging scores and c) a clinical database comprising a third plurality of anatomical neuroimaging scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category for a drug formulation, wherein the probability response category is selected from the group comprising sensitive, intermediate and resistive. In one embodiment, the patient outcome measure comprises a CGI score. In one embodiment, the method further comprises treating the patient classified within the probability response category selected from the group comprising sensitive and intermediate with a pharmaceutical formulation comprising an anticonvulsant and a neuroactive modulator, wherein at least one symptom of the nervous system disorder is reduced. In one embodiment, the anatomical neuroimaging score comprises data from methods including, but not limited to, ultrasound, X-ray, radionulcide scanning, CAT, MRI, LORETA or VARETA. In one embodiment, the anatomical neuroimaging score includes, but is not limited to, multivariate Z scores, univariate Z scores (i.e., standard deviations), probability scores and raw data. In one embodiment, the nervous system disorder includes, but is riot limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent and a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Still yet another further advantage of the present invention contemplates a method, comprising: i) providing; a) a convalescent population database comprising a first plurality of objective symptom measurement scores and a patient outcome measure; b) a normative population database comprising a second plurality of objective symptom measurement scores; and c) a clinical database comprising a third plurality of objective symptom measurement scores derived from an individual patient exhibiting at least one symptom of the nervous system disorder; ii) comparing the individual patient scores with the normative database such that an abberant individual patient score is identified; and iii) comparing the abberant individual patient score with the convalescent database such that the patient is classified within a probability response category, wherein the probability response category is selected from the group comprising sensitive, intermediate and resistive. In one embodiment, the patient outcome measure comprises a CGI score. In one embodiment, the method further comprises treating the patient classified within the probability response category selected from the group comprising sensitive and intermediate with a pharmaceutical formulation comprising an anticonvulsant and a neuroactive modulator, wherein at least one symptom of the nervous system disorder is reduced. In one embodiment, the objective symptom measurement score is determined by a method including, but not limited to, Actigraph or self-report questionnaires. In one embodiment, the objective symptom measurement score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Still yet another further advantage of the present invention contemplates a method, comprising: i) providing; a) a convalescent database comprising a first plurality of multi-modality measurement scores and a patient outcome measure; b) a normative database comprising a second plurality of multimodality measurement scores; and c) a clinical database comprising a third plurality of multimodality measurement scores derived from an individual patient exhibiting at least one symptom of a nervous system disorder; ii) comparing the individual patient scores with the normative database such that an abberant individual patient score is identified; and iii) comparing the abberant individual patient score with the convalescent database such that the patient is classified within a probability response category, wherein the probability response category is selected from the group comprising sensitive, intermediate and resistive. In one embodiment, the patient outcome measure comprises a CGI score. In one embodiment, the method further comprises treating the patient classified within the prognosis category selected from the group comprising sensitive and intermediate with a pharmaceutical formulation comprising an anticonvulsant and the neuroactive modulator, wherein at least one symptom of the nervous system disorder is reduced. In one embodiment, the multimodality measurement scores are determined by combined methodologies including, but not limited to, electroencephalographic/heart rate, electroencephalographic/blood pressure, electroencephalographic/electrophysiological, electroencephalographic/biological etc. In one embodiment, the multimodality measurement score includes, but is not limited to, multivariate Z scores, univariate Z scores (i.e., standard deviations), probability scores and raw data. In one embodiment, the nervous system disorder includes, but is not limited to, at least one neurobehavioral or intrapulmonary, neuropsychological, neurophysiological or behavioral or intrapulmonary symptom. In one embodiment, the anticonvulsant comprises oxcarbazepine. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor comprises a monoaminergic reuptake inhibitor. In one embodiment, the monoaminergic reuptake inhibitor comprises bupropion.

Yet another advantage of the present invention contemplates a method, comprising: i) providing; a) a convalescent population database comprising a first plurality of neuroelectrical scores and a patient outcome measure; b) a normative population database comprising a second plurality of neuroelectrical scores; and c) a clinical database comprising a third plurality of neuroelectrical scores derived from an individual patient exhibiting at least one symptom of a nervous system disorder; ii) comparing the individual patient scores with the normative database such that an abberant individual patient score is not identified; and iii) excluding said individual patient from comparing said third plurality of neuroelectrical scores with said convalescent database.

Yet another advantage of the present invention contemplates a method, comprising: i) providing; a) a convalescent population database comprising a first plurality of neuroelectrical scores and a patient outcome measure; b) a normative population database comprising a second plurality of neuroelectrical scores; and c) a clinical database comprising a third plurality of neuroelectrical scores derived from an individual patient exhibiting at least one symptom of a nervous system disorder; ii) comparing the individual patient scores with the normative database such that an abberant individual patient score is identified; and iii) comparing the abberant individual patient score with the convalescent database under conditions that identify a formulation having an effacacious response for said nervous system disorder.

Another advantage of the present invention contemplates a device, comprising: i) a platform having a plurality of compartments wherein the compartments contain at least one pharmaceutical formulation comprising an anticonvulsant and a neuroactive modulator; ii) an aperture extending through the platform configured to align with one of the compartments thus dispensing the formulation from the compartment; iii) an advancing mechanism connected to the platform wherein the platform is translocated such that the formulation becomes aligned with the aperture; and iii) an exterior coding system marked on the compartments wherein each compartment is uniquely identified. In one embodiment, the platform is circular. In another embodiment, the platform is square. In another embodiment, the platform is rectangular. In another embodiment, the platform is cylindrical. In one embodiment, the pharmaceutical formulation further comprises a third drug. In one embodiment, the third drug includes, but is not limited to, selective serotonergic reuptake inhibitors, antipsychotics, antianxiety agents, barbiturates, antiparkinsonians, analgesics, cardiac drugs, stimulants, monoamine oxidase inhibitors or nutraceuticals. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor is a monoaminergic reuptake inhibitor. In another embodiment, the formulation comprises the selective serotonin reuptake inhibitor, the oxcarbazepine and the monoaminergic reuptake inhibitor. In one embodiment, the formulation comprises a compounded formulation. In another embodiment, the compounded formulation further comprises sertraline. In one embodiment, the formulation and/or compounded formulation includes, but is not limited to, a tablet, bi-layer tablet, tri-layer tablet, capsule, an oral liquid, intrapulmonary liquid, aerosol, bi-compartment capsule, tri-compartment capsule, and fast-dissolve compound.

Another advantage of the present invention contemplates a device comprising a blister package containing a plurality of pharmaceutical formulations. In one embodiment, the blister package comprises a dome structure that retains a pharmaceutical formulation on the surface of a backing material. In one embodiment, an unadministered pharmaceutical formulation is visible within the blister package following the indicated administration day. In one embodiment, a blister package comprises a single formulation or a plurality of formulations capable of identifying administration on a daily basis. In one embodiment, blister packages organize identical tablets by rows. In another embodiment, the row organization of identical tablets are marked on the backing comprising a coding system that results in the specific identification of each formulation present on the blister package. In one embodiment, the blister package comprises a coding system that references days, months, and years. In one embodiment, the pharmaceutical formulation further comprises at least one third drug. In one embodiment, the third drug includes, but is not limited to, selective serotonergic reuptake inhibitors, antipsychotics, antianxiety agents, barbiturates, antiparkinsonians, analgesics, cardiac drugs, stimulants, monoamine oxidase inhibitors or nutraceuticals. In one embodiment, the neuroactive modulator includes, but is not limited to, a neurotransmitter reuptake inhibitor, a neurotransmitter receptor agent or a neurotransmitter metabolic inhibitor. In one embodiment, the neurotransmitter reuptake inhibitor is a monoaminergic reuptake inhibitor. In another embodiment, the formulation comprises the selective serotonin reuptake inhibitor, the oxcarbazepine and the monoaminergic reuptake inhibitor. In one embodiment, the formulation comprises a compounded formulation. In another embodiment, the compounded formulation further comprises sertraline. In one embodiment, the form of the formulation includes, but is not limited to, a tablet, bi-layer tablet, tri-layer tablet, capsule, oral liquids, intrapulmonary liquids, aerosol, bi-compartment capsule, tri-compartment capsule, and fast-dissolve compound.

Definitions

The terminology used herein is intended for interpretation according to common usage and definition in the related art, in addition to specific clarifications regarding the following:

The term “nervous system disorder”, as used herein, refers to any psychiatric disorder or neurological disorder.

The term “psychiatric disorder”, as used herein, refers to any abnormal central or peripheral nervous system condition defined and classified in the DSM IV. For example, such “nervous system disorders” include, but are not limited to: i) Disorders Usually First Diagnosed in Infancy, Childhood, or Adolescence; ii) Cognitive Disorders; Mental Disorders Due to a General Medical Condition; iii) Substance-Related Disorders; iv) Schizophrenia and Other Psychotic Disorders; v) Mood Disorders; vi) Anxiety Disorders; vii) Somatoform Disorders; Factitious Disorder; Dissociative Disorders; viii) Sexual and Gender Identity Disorders; ix) Eating Disorders; Sleep Disorders; x) Impulse-Control Disorders Not Elsewhere Classified; Adjustment Disorder; or xi) Personality Disorders. In one embodiment, a “psychiatric disorder” comprises a “neurobehavioral or intrapulmonary disorder”. In another embodiment, a “psychiatric disorder” comprises a “neurophysiological disorder”.

The term “neurobehavioral or intrapulmonary disorders”, as used herein, refers to any medically relevant condition significantly involving either the peripheral or central nervous system. The present invention specifically contemplates, but is not limited to, neurobehavioral or intrapulmonary disorders such as delusions, schizophrenia, affective disorders, neuroses, psychoses, anxiety, chemical dependency, eating disorders (i.e., for example bulimia and anorexia), attention deficit disorder, attention deficit hyperactivity disorder, and other similar conditions as defined in the current DSM-IV and future editions.

The term “neurophysiological disorder”, as used herein, refers to any condition comprising abnormal behavior, abnormal cognition and/or abnormal movement that has an identifiable physiological basis.

The term “neurological disorder” or “neurological disease”, as used herein, refers to any debilitating mental or physical condition involving symptomology related to motor function, cognitive function, cognition and/or pain. In one embodiment, the primary etiology of the neurological disorder comprises either the peripheral or central nervous system. Specifically, neurological disorders are contemplated as including, but not limited to, alzheimer's, epilepsy, parkinson's, huntington's, dyslexia, migraine, pain, neuropathy, stroke, or facial nerve lesions.

The term “anticonvulsants”, as used herein, refers to a pharmaceutical compound that affects either the central or peripheral nervous system to protect against spontaneous and uncontrollable depolarization. In one embodiment, “anticonvulsants” include, but are not limited to, acetazolamide, apo-carbamazepine, apo-diazepam, apo-lorazepam, apo-primidone, ativan, carbamazepine, oxcarbazepine, clobazam, clonazapam, depakene, depakote, diamox, diazemuls, diazepam, dilantin, diphenylhydantoin, divalproex sodium, epitol, epival, ethotoin, ethosuximide, felbamate, frisium, gabapentin, keppra, klonopin, lamictal, lamotrigine, levetiracetam, lorazepam, mazepine, mogadon, myidone, mysoline, neurontin, nitrazepam, novocarbamaz, novo-lorazepam, nu-loraz, paraldehyde, phenobarbital, mephobarbital, phenytoin, mephenytoin, phenacemide, primidone, progabide, pyridoxine, pyridoxine hydrochloride, rivotril, sabril, sertan, sodium valproate, tegretol, tiagabine, topamax, topiramate, trimethadione, trileptal, valium, valproate sodium, valproic acid, vigabatrin, vitamin B6 or vivol.

The term “neuroactive modulator”, as used herein, refers to any compound that modifies neuronal activity. The term “neuroactive modulator” includes, but is not limited to, neurotransmitter reuptake inhibitors, neurotransmitter receptor agents, or neurotransmitter metabolic inhibitors.

The term “neurotransmitter”, as used herein, refers to any compound comprising the following properties: i) localization in the pre-synaptic terminal; ii) synthesized in the neuron; iii) released upon neuronal depolarization; iv) presence of specific post-synaptic receptors that produce electrical potentials; and v) interruption of neurotransmitter synthesis, release or receptor activation prevents normal intercellular communication.

The term “neurotransmitter reuptake inhibitors”, as used herein, comprises any chemical or peptide that reduces the ability of a pre- or postsynaptic membrane to remove neurotransmitter compounds from the synaptic cleft. For example, neurotransmitter reuptake inhibitors may effect neurons including, but not limited to, monoaminergic, glycinergic, glutaminergic or GABAergic neurons.

The term “monoaminergic reuptake inhibitors”, as used herein, comprises any chemical or peptide having a free amine substituent that acts on the pre- or postsynaptic membrane that blocks the transport of a monoaminergic neurotransmitter from the synaptic cleft into the neuron. Monoaminergic neurotransmitters effected by these inhibitors include, but are not limited to, norepinephrine, dopamine and serotonin. In one embodiment, “monoaminergic reuptake inhibitors” comprise “noradrenergic reuptake inhibitors” that include, but are not limited to, imipramine, amitryptyline, desipramine, clomipramine, desmethyldlomipramine, nortryptyline, doxepine, protryptyline, maprotiline, nisoxetine, tomoxetine, reboxetine, viloxazine, or lofepramine. In another embodiment, “monoaminergic reuptake inhibitors” comprise “dopaminergic reuptake inhibitors” that include, but are not limited to, maxindol, cocaine, nomefensine, amineptine, medifoxamine, GBR12909, GBR12783 and GBR13069. In another embodiment, “monoaminergic reuptake inhibitors” comprise “noradrenergic/serotonergic reuptake inhibitors, including, but not limited to venlafaxine, milnacipran and duloxetine. In another embodiment, “monoaminergic reuptake inhibitors” comprise “selective serotonergic reuptake inhibitors” (i.e., SSRIs) that include, but are not limited to, fluoxetine, sertraline, citalopram, paroxetine, fluvoxamine, nefazodone, hyperforin or Ro-15-8081.

The term “glutaminergic reuptake inhibitors”, as used herein, refers to any compound or peptide that is capable of reducing the amount of glutamate that is removed from the synaptic cleft by a pre- or postsynaptic membrane. In one embodiment, “glutaminergic reuptake inhibitor” include, but are not limited to, aminocaproic acid or lithium carbonate.

The term “glycine reuptake inhibitors”, as used herein, refers to any compound or peptide that is capable of reducing the amount of glycine that is removed from the synaptic cleft by a pre- or postsynaptic membrane. In one embodiment, “glycine reuptake inhibitors” include, but are not limited to, ALX 5407, sarcosine, or 5,5-diaryl-2-amino-4-pentenoates.

The term “GABA reuptake inhibitors”, as used herein, refers to any compound or peptide that is capable of reducing the amount of gamma-amino-butyric acid (GABA) that is removed from the synaptic cleft by a pre- or postsynaptic membrane. In one embodiment, “GABA reuptake inhibitors” include, but are not limited to, tiagabine.

The term “neurotransmitter receptor agents”, as used herein, refers to any compound that modifies the postsynaptic binding efficacy of a neurotransmitter. “Neurotransmitter receptor agents” include, but are not limited to, monoamine receptor agents, acetylcholine receptor agents, glycine receptor agents, GABA receptor agents or NMDA receptor agents.

The term “monoamine receptor agents”, as used herein, refers to any compound that modifies the postsynaptic binding efficacy of monoaminergic neurotransmitters. The monoaminergic neurotransmitters include, but are not limited to, norepinephrine, dopamine or serotonin. “Monoamine receptor agents” include, but are not limited to, clonidine, dopamine, dobutamine, prenalteraol, xamoterol, propranolol, atenolol, betaxolol, nadolol, carvedilol, sotolol, timolol, labetolol, acebutolol, pindolol, esmolol, metoprolol, bisoporol, bucindolol, mexiletine, phenoxybenzamine, pindolol, flexinoxan, sunepitron, buspirone, azapirone, gepirone, ipsapirone, 8-hydroxy-2-(di-n-propylamino)tetralin, lissuride, roxindole, salbutamol, clenbuterol, SR58611A, M100907, ORG 5222, U-101387, methysergide, cyproheptadine, metergoline, ritanserin, trazodone, nefazodone, carbodopa, levodopa, mianserin, imidazoline, idazoxan, benzodioxinopyrrole, fluparoxan, R47243, iloperidone, benzamide, amisulpride, sulpiride, flupenthixol, haloperidol, fluphenazine, zuclopenthixol, risperidone, ziprasidone, sertindole, melperone, perphenazine, chlorpromazine, levomepromazine, quetiapine, thioridazine, clozapine, zotepine or olanzapine.

The term “acetylcholine receptor agents”, as used herein, refers to any compound that modifies the postsynaptic binding efficacy of cholinergic neurotransmitters. “Acetylcholine receptor agents” include, but are not limited to, carbachol, methacholine, bethanechol, pilocarpine, arecholine, nicotine, nicotinic alkaloids, muscarine, alpha-latrotoxin, atropine, benzotropine, hyoscyamine, ipratropium, scopolamine, trihexyphenidyl, botulinum toxin, alpha-bungarotoxin, d-tubocurarine, methotramine, mecamylamine or pirenzepine.

The term “glycine receptor agents”, as used herein, refers to any compound that modifies the postsynaptic binding efficacy of glycinergic neurotransmitters. “Glycine receptor agents” include, but are not limited to, glycine, beta-alanine, taurine, d-cycloserine, strychine, (+/−)-3-amino-1-hydroxy-2-pyrrolidone, 1-aminocyclopropanecarboxylic acid, 2-aminostrychnine, RU-5135, 5,6,7,8-tetrahydro-4H-isoxazolo[5,4-c]azepin-3-ol, norharmane, or PK-8165.

The term “GABA receptor agents”, as used herein, refers to any compound that modifies the postsynaptic binding efficacy of GABAergic neurotransmitters. “GABA receptor agents” includes, but are not limited to, baclofen, bicuculline, pagoclone, benzodiazepines, chloride ion or barbiturates.

The term “NMDA receptor agents”, as used herein, refers to any compound that modifies the postsynaptic binding efficacy of glutamate. “NMDA receptor agents” include, but are not limited to, glutamate, 2-amino-7-phosphoheptanoic acid (i.e., binding at the glycine regulation site), carbamazepine, tacrine, phencyclidine, ketamine, dizolcipine, N-methyl-d-aspartic acid (NMDA), MK-801, LY-215490, LY-274614, LY-233536, LY-215490, LY-233053, LY-293558, ibotenate, (tetrazol-5-yl)-glycine, 4-methylene-L-glutamate, D-AP5, D-AP7, (R)-4-Oxo-AP5 or CGS 19755.

The term “neurotransmitter metabolic inhibitors”, as used herein, refers to any compound that interfers with synthetic or degradative enzymes of a neurotransmitter. In should be understood that the enzymes may be located either intra- or extracellularly. “Neurotransmitter metabolic inhibitors” include, but are not limited to, monoamine metabolic inhibitors, acetylcholine metabolic inhibitors, glutamate metabolic inhibitors, glycine metabolic inhibitors or GABA metabolic inhibitors.

The term “monoamine metabolic inhibitors”, as used herein, refers to any compound that interfers with synthetic or degradative enzymes of monoaminergic neurotransmitters. The monoaminergic neurotransmitters include, but are not limited to, norepinephrine, dopamine or serotonin. “Monoamine metabolic inhibitors” include, but are not limited to, “monoamine oxidase inhibitors” and “catechol-O-methyltransferase inhibitors”.

The term “catechol-O-methyltransferase inhibitors”, as used herein, refers to any compound that interfers with the synthesis of the enzyme, catechol-O-methyltransferase. In one embodiment, “catechol-O-methyltransferase inhibitors” include, but are not limited to, tolcapone or entacapone.

The term “acetylcholine metabolic inhibitors”, as used herein, refers to any compound that interfers with the synthetic or degradative enzymes of cholinergic neurotransmitters. “Acetylcholine metabolic inhibitors” include, but are not limited to, neostigmine, edrophonium, ambenonium, physostigmine, pyridostigmine, tacrine, donepezil, rivastigmine, oxotremorine, epibatidine, organophosphates or nerve gas.

The term “GABA metabolic inhibitors”, as used herein, refers to any compound that interfers with the synthetic or degradative enzymes of GABAergic neurotransmitters. “GABA metabolic inhibitors” include, but are not limited to, vigabatrin.

The term “third drug”, as used herein, refers to any pharmaceutical formulation prescribed for the treatment of any clinically diagnosed disease or medical condition. In one embodiment, a third drug includes, but is not limited to, selective serotonin reuptake inhibitors, monoamine oxidase inhibitors, antipsychotic drugs, antianxiety/anxiolytic drugs, barbiturates, stimulants, antiparkinsonian drugs, analgesic drugs, cardiac agents, or nutriceuticals.

The term “monoamine oxidase inhibitors”, as used herein, refers to any compound that is capable of inhibiting the enzyme monoamine oxidase that results in an elevated synaptic level of monoamine neurotransmitters. In one embodiment, “monoamine oxidase inhibitors” include, but are not limited to, pargyline, lazabemide, sufinaminde, selegiline, moclobemide, brofaromine, befloxatone, clorgyline, phenelzine, nialamide or tranylcypromine.

The term “antipsychotic drugs”, as used herein, refers to any substance that lessens the symptoms of a psychotic disorder. In one embodiment, “antipsychotic drugs” include, but are not limited to, acetophenazine, benzamide amisulpride, buspirone, chlorprothizene, thiothizene, sulpiride, amisulpride, flupenthixol, haloperidol, fluphenazine, zuclopenthixol, risperidone, ziprasidone, sertindole, melperone, perphenazine, promazine, pimozide, meprobamate, mesoridazine, molindone, trazodone, chlorpromazine, triflupromazine, trifluoperazine, levomepromazine, lithium carbonate, loxapine, quetiapine, thorazine, thioridazine, clozapine, zotepine or olanzapine.

The term “antianxiety/anxiolytic drugs”, as used herein, refers to any substance that lessens the symptoms of anxiety. In one embodiment, the “antianxiety/anxiolytic drugs” include, but are not limited to, alprazolam, chlordiazepoxide, clonazepam, chlorazepate, diazepam, halazepam, flurazepam, lorazepam, oxazepam, temazepam, and triazolam.

The term “barbiturates”, as used herein, refers to any compound comprising a barbiturate ring structure. In one embodiment, “barbiturates” include, but are not limited to, amobarbital, aprobarbital, butabarbital, butalbital, mephobarbital, pentobarbital, phenobarbital, secobarbital and talbutal.

The term “stimulants”, as used herein, refers to any substance that increases cognitive capability. In one embodiment, “stimulants” include, but are not limited to, amphetamine, dextroamphetamine, methamphetamine, modafinil (Provigil), methylphenidate, atomoxetine, ephedrine, caffeine, theophylline or theobromine.

The term “antiparkinsonian drugs”, as used herein, refers to any substance that reduces at least one symptom of parkinson's disease. In one embodiment,“antiparkinsonian drugs” include, but are not limited to, levodopa, carbidopa, benserazide, amantadine, apomorphine, dopamine, pergolide, bromocriptine, lisuride, benzotropine, trihexyphenidyl, procyclidine, biperiden, ethopropazine, and diphenydramine.

The term “analgesic drugs”, as used herein, refers to any substance that reduces the perception of pain. In one embodiment, “analgesic drugs” include, but are not limited to, heroin, hydromorphone, oxymorphone, levorphanol, methadone, meperidine, fentanyl, codeine, hydrocodone, drocode, oxycodone, propoxyphene, buprenorphine, pentazocine, nalbuphine, butrophanol, salicyclic acid, aspirin, methyl salicylate, diflunisal, salsolate, apazone, acetaminophen, phenacetin, acetanilide, aniline, indomethacin, sulindac, mefenamic acid, meclofenamate, tolmetin, ibuprofen, naproxen, fenoprofen, ketoprofen, flurbiprofen, piroxicam, diclofenac, etodolac, nabumetone, aurothioglucose, gold sodium thomalate, auranofin, ergotamine, dihydroergotamine, ergonovine, ergoloid and bromocriptine.

The term “cardiac agents”, as used herein, refers to any substance that improves the performance of cardiac tissue. Cardiac performance may be improved by increasing or decreasing contractility or by synchronization of electrical potentials. In one embodiment, “cardiac agents” include, but are not limited to, digoxin, dopamine, dobutamine, prenalteraol, xamoterol, propranolol, atenolol, betaxolol, nadolol, carvedilol, sotolol, timolol, labetolol, acebutolol, pindolol, esmolol, metoprolol, bisoporol, bucindolol, mexiletine, phenoxybenzamine, pimobendan, sulmazole, levosimendan, dihydropyridine, amlodipine, mibefradil, vesnarinone, verapamil, nifedipine, nisoldipine, nicardipine, felodipine, isradipine, beperidil, amlodipine, lidocaine, phenytoin, procainamide, amioradone, bretylium, quinidine, disopyramide, amiodarone, flecainide, encainide, propafenone, magnesium, amrinone, milrinone, enoximone, piroximone, sulmazole, pimobendan, spironolactone, hydralazine, isosorbide dinitrate, nitroglycerin, endothelin-1, nitric oxide, candesartan, irbesartan, losartan or valsartan.

The term “nutriceuticals”, as used herein, refers to any substance that relies on natural products and/or remedies to treat nervous system disorders. In one embodiment, “nutriceuticals” may include, but are not limited to, amino acids, fatty acids and unisolated plant products, either alone or in combination. In another embodiment, “nutriceuticals” includes, but is not limited to, Tryptophane-Phenylalanine-Glutamine, ginko biloba, essential fatty acid omega 3, essential fatty acid omega 6 or essential fatty acid omega 9.

The term “symptom” or “symptoms”, as used herein, refers to any physical, mental or emotional manifestation that is characteristic in the differential diagnosis of a particular medical condition. For example, the symptomology of diseases and other medical conditions are compiled in publications such as the International Classification of Diseases (ICD) and the Diagnostic and Statistical Manual of Mental Disorders-IV (DSM-IV).

The term “delusion”, as used herein, refers to any mental condition that results in the perception of an altered reality. Specifically, delusion is contemplated to be, but not limited to, “delusions of grandeur”, psychoses or hallucinations.

The term “schizophrenia”, as used herein, refers to any idiopathic psychosis characterized by chronically disordered thinking and emotional withdrawal often associated with paranoid delusions and auditory hallucinations.

The term “mood disorder”, as used herein, refers to any mental condition that results in behavior patterns representing alterations in mood. Specifically, mood disorders are contemplated to be, but not limited to, unipolar depression or bipolar depression.

The term “personality disorder”, as used herein, refers to any condition, that may or may not respond to medical intervention, that include perversion and chronic dysfunction appearing in multiple forms during a patient's life. In one embodiment, characteristic symptoms include, but are not limited to, avoidance, paranoia, withdrawal and dependency. More generally, another embodiment reflects a pattern of behavior such as, but not limited to, chemical dependency, deviant eating patterns, hypochondriasis or antisocial behavior.

The term “deviant eating patterns”, as used herein, refer to any condition wherein a compulsive behavior pattern results in a significant increase or decrease in food consumption. Specifically, the present invention contemplates, but is not limited to, conditions such as bulimia and anorexia nervosa.

The term “depression”, as used herein, refers to any nervous system disorder and/or mental condition characterized by, but not limited to, the following symptoms: withdrawal, insomnia, hypersomnia, loss of appetite, altered daily rhythms of mood, activity, temperature and neuroendocrine function. For example, dsythymia, seasonal affective disorder and the like.

The term “neuroses”, as used herein, refers to any mild psychiatric disorder wherein the ability to comprehend is retained but suffering and disability are very severe. Other characteristics of neuroses include, but are not limited to, mood changes (i.e., for example, anxiety, panic, dysphoria) or limited abnormalities of thought (i.e., for example, obsessions, irrational fears) or of behavior (rituals or compulsions, pseudoneurological or hysterical conversion signs).

The term “psychoses”, as used herein, refers to any severe psychiatric disorder wherein there is a marked impairment of behavior, a serious inability to think coherently, or to comprehend reality. Psychoses may include organic conditions associated with a definable toxic, metabolic, or neuropathologic change characterized by confusion, disorientation, memory disturbances and behavioral or intrapulmonary disorganization.

The term “anxiety state”, as used herein, refers to any human emotion, closely allied with appropriate fear, often serving psychobiologically adaptive purposes that is a cardinal symptom of many psychiatric disorders. Specifically, anxiety is commonly associated with, but not limited to, neurotic depression, panic disorder, phobias, obsessive-compulsive disorders and other related personality disorders.

The term “patient”, as used herein, refers to any mammal, human or animal, that may benefit from the administration of a pharmaceutical compound.

The term “formulation” or “pharmaceutical formulation”, as used herein, refers to any composition intended for the administration of a pharmaceutical compound, or combination, including, but not limited to, any chemical or peptide, natural or synthetic, that is administered to a patient for medicinal purposes. Specifically, a formulation may comprise either a single compound or a plurality of compounds.

The term “compounded” or “compounded formulation”, as used herein, refers to any formulation containing a plurality of compounds, wherein the compounds may have the same, or different dosage ratios, and further wherein the compounds may be uniform (i.e., evenly mixed) or non-uniform (i.e., unevenly mixed, including but not limited to, separated tablet layers or separated capsule compartmentalization).

The term “tablets”, as used herein, refers to any solid formulation comprising at least one pharmaceutical compound intended for oral or intrapulmonary administration to a patient. In one embodiment, “tablets” may have multiple layers (i.e., multilayered tablets), wherein each layer comprises different pharmaceutical formulation.

The term “capsules”, as used herein, refers to any polymer film-based container comprising a single or plurality of compartments containing at least one pharmaceutical compound intended for oral or intrapulmonary administration to a patient. In one embodiment, “capules” may bave multiple compartments (i.e., multi-compartmentalized), wherein each compartment comprises a different pharmaceutical formulation.

The term “oral liquids”, as used herein, refers to any pourable composition that is absorbed by the gastrointestinal system (i.e., mouth, throat, stomach, intestines etc.).

The term “intrapulmonary liquids”, as used herein, refers to any pourable composition that is absorbed by the pulmonary system, (i.e., for example, the trachea, bronchial tree, alevoli and the like). In one embodiment, “intrapulmonary liquids” are administered to a patient using devices including, but not limited to, an intratracheal catheter or other pulmonary intubation system known to those having skill in the art.

The term “transdermal patches”, as used herein, refers to any sheet of material comprising at least one pharmaceutical compound intended for topical administration to a patient.

The term “polymer-coated tablets”, as used herein, refers to any exterior layer adhered to the surface of a tablet. Primarily, these exterior layers prevent gastrointestinal degradation (i.e., enteric coatings) or provide a mechanism for timed-release or sustained release formulations.

The term “liposomes”, as used herein, refers to any spherical composition comprising a lipid bilayer membrane that may, or may not, encase other compounds.

The term “microspheres”, as used herein, refers particularly to substantially spherical particles which can be a monolithic solid sphere or a small capsule. Microspheres preferably have a mean diameter of between 0.5 and 250 μm, preferably between 10 μm and 150 μm and more preferably between 10 and 100 μm as measured using a conventional light microscope.

The term “aerosols”, as used herein, refers to the administration of any drug to a patient by a mist or spray comprising liquid droplets. In one embodiment, the aerosol is administered intra-nasally and contacts the nasal passages including, but not limited to, the nasal sinus membranes. In another embodiment, the aerosol is administered intrapulmonarly and contacts pulmonary tissue (i.e., for example, the alevoli).

The term “fast-dissolving compounds”, as used herein, refers to any composition that dissolves or dissolutes in the oral or intrapulmonary cavity, and is absorbed through the sublingual membranes, within five minutes.

The term “sterile injectable solutions”, as used herein, refers to any composition that is suitable for delivery by direct dilution in the bloodstream of a patient.

The term “refractory”, as used herein, refers to any diagnosed psychological condition or symptom that is not alleviated following the administration of at least one pharmaceutical compound at a dose expected by one skilled in the art to be therapeutically effective.

The term “non-remissive”, as used herein, refers to a condition where a patient has not undergone any reduction of at least one symptom of a nervous system disorder. A non-remissive condition may result whether, or not, the patient has been administered a pharmaceutical or a nutriceutical compound (i.e., as a third or polytherapeutic regimen). In one embodiment, a non-remissive condition comprises a patient that has been administered a pharmaceutical or nutriceutical compound and has undergone an insignificant reduction of at least one symptom of a nervous system disorder.

The term “exhibiting”, as used herein, refers to any physical, mental or emotional expression of any symptom of any medical condition by a patient.

The term “sequentially”, as used herein, refers to any significant administration of one pharmaceutical or nutriceutical formulation prior to initiating the administration of a subsequent pharmaceutical or nutriceutical compound. In one embodiment, a plurality of formulations are sequentially administered within forty-eight hours, preferably within twenty-four hours and more preferably within twelve hours.

The term “divided daily dose”, as used herein, refers to any total quantity per day of a pharmaceutical or nutriceutical compound prescribed by medical personnel for any diagnosed condition, wherein the total quantity may be distributed in smaller, equal, doses throughout the day. The “divided daily dose” of two or more sequential formulations may be expressed by the term “divided daily dose ratio”, wherein each number represents the milligram divided daily dose of one formulation given on a particular day. For example, a formulation comprising oxcarbazapine and bupropion having a divided daily dose ratio of 4500/750 means that during a twenty-four hour period 4500 mg of oxcarbazepine and 750 mg of bupropion are administered to a patient.

The term “neuroelectrical”, as used herein, refers to information collected by any electroencephalographic analysis (abbreviated as EEG) as used herein, comprising any method, recognized in the art of neurology, to record brain wave patterns.

The term “artifact-free”, as used herein, refers to the collection of any neuroelectrical data that contains exclusively only information reflective of the functioning of the nervous system.

The term “absolute power”, as used herein, refers to any measure of the strength of brain electrical activity.

The term “relative power”, as used herein, refers to any measure of how brain activity is distributed.

The term, “symmetry”, as used herein, refers to any measure of the balance of the observed brain activity between hemispheres.

The term, “coherence”, as used herein, refers to any measure of the coordination of the observed brain activity.

The term, “frequency”, as used herein, refers to the average frequency of any electrical power within any major frequency band (i.e., for example, delta, theta, alpha or beta frequency bands).

The term “raw data”, as used herein, refers to any single number or score, that results from an administration of a quantitative testing procedure. Raw data scores act to rank order a patient's response or performance for comparison to others who have undergone the same testing procedure. Further, raw data scores may be subjected to various statistical calculations known in the art to produce probability score statements such as, but not limited to, univariate analysis or multivariate analysis.

The term “univariate score” or “probability score”, as used herein refers, to any single number, based on a statistical analysis of raw data scores, that reflect the relationship of a specific patient to any one particular group of individuals. For example, a univariate Z score is analogous to the statistical definition of standard deviation that determines the distribution of a data population around the mean value.

The term “multivariable Z score”, “multivariate Z score” or “composite Z score”, as used herein, refers to any single number, based on quantitative multivariate analysis, which reflects the overall statistical assessment of an individual patient's clinical condition based upon an integrated statistical calculation of a plurality of qualitatively unique univariate Z scores and/or raw data scores.

The term “database”, as used herein, refers to any organized collection of quantitative measurements comprising scores unique to an identified population. It is expected to be understood by those skilled in the art that a database may further comprise clinical observations either with or without associated non-parametric classification scores.

The term “probability response category”, as used herein, refers to any set of delimiting quantitative predictors (i.e., for example, QEEG scores, psychometric test battery scores, biological indicator scores etc.) that are associated with the probability of a significant response when following a specific course of treatment. For example, a probability response category may be, but not limited to; i) “sensitive” if an individual patient's clinical data scores are classified within a population having at least an 80% probability of a significant response with a specific pharmaceutical formulation, a “sensitive” category may be further subdivided into various levels (i.e., for example, Level 1 showing a 100-90% probability and Level 2 showing a 90-80% probability); ii) “intermediate” if an individual patient's clinical data scores are classified within a population having between approximately 20% -80% probability of a significant response with a specific pharmaceutical formulation, an “intermediate” category may be further subdivided into various levels (i.e., for example, Level 3 showing a 80-65% probability, Level 4 showing a 65-50% probability, Level 5 showing a 50-35% probability and Level 6 showing a 35-20% probability); and iii) “resistive” if an individual patient's clinical data scores are classified within a population having less than a 20% probability of a significant response with a specific pharmaceutical formulation, a “resistive” category may be further subdivided into various levels (i.e., for example, Level 7 showing a 20-10% probability and Level 8 showing a 10-0% probability).

The term, “patient outcome measure”, as used herein, refers to any clinical information that signifies a patient response to a pharmaceutical therapy regimen. For example, an outcome measure may include, but is not limited, to a Clinical Global Improvement score, qualitative non-parametric assessments or written annotations.

The term, “significant response”, as used herein, refers to any patient exhibiting a change in Clinical Global Improvement (CGI) of two (2) levels or more as a result of a pharmaceutical therapy regimen.

The term, “CGI score”, as used herein, refers to a quantitative assessment of patient response based upon the level of response to a pharmaceutical therapy regimen based upon a Clinical Global Improvement. One of skill in the art should recognize that the Clinical Global Improvement scale as used herein is similar, but not identical, to the Cognitive Global Impression scale.

The term “population”, as used herein, refers to any group of individuals selected for comparison to another population or single individual.

The term “convalescent population”, as used herein, refers to any group of persons having clinical improvement of a specific clinical condition subsequent to a specific formulation or combination of formulations.

The term “normative population”, as used herein, refers to any group of persons that have not been treated for any specific clinical condition.

The term “individual patient score”, as used herein, refers to any clinical measurement or determination having relevance to the expression of a symptom of a disease or medical condition.

The term “abberant”, as used herein, refers to any clinical variable that is outside of a normally considered normal range. In one embodiment, “abberant” refers to any value for a test for which similar values of a convalescent database show frequency of responses of medication(s) that are higher or lower than the background (i.e., random chance) rate of responsivity.

The term “psychometric test battery”, as used herein, refers to any written, oral or intrapulmonary, tactile or visual stimulus wherein the response of the patient is recorded. A comparison and analysis of all responses in a test battery provide medical personnel with information for a diagnosis and prognosis of any disease or medical condition.

The term “biological indicator”, as used herein, refers to any specific chemical or other biochemical compound, either organic or protein, that provides information for diagnosis and prognosis of any disease or medical condition when sampled from fluids or tissues of a patient.

The term “brain cognitive indicator”, as used herein, refers to any metabolic assay that measures the activity level of a central neuron. For example, a metabolic assay may include, but not be limited to, glucose utilization or radiolabeled medicines (i.e., dopamine tags).

The term “glucose utilization”, as used herein, refers to the measurement of the metabolism of glucose in central nervous system neurons as a measure of brain activity. Glucose utilization may be used as a cognitive indicator as a predictor of overall cognitive function.

The term “radiolabeled medicines”, as used herein, refers to the activity measurement of biochemical pathways by a substrate of the pathway comprising a radioactive label. Such a compound may, for instance, accumulate at a particular step in a biochemical pathway such that it rate of appearance is reflective of biochemical activity.

The term “genotype allelic profile”, as used herein, refers to any specific combination of genes, reflecting the known biodiversity within the genes, which are responsible for symptomology, or lack thereof, in a patient that provides information for diagnosis and prognosis of any disease or medical condition.

The term “brain neuroimaging”, as used herein, refers to any method that results in a graphical presentation of the morphological and anatomical structure of the central nervous system. The methods may include, but are not limited to, positron emission transmission (PET), magnetic resonance imaging (MRI), functional magnetic resonance imaging (fMRI), single photon emission computed tomography (SPECT), X-ray using deoxyglucose-6 phosphate, low resolution emission tomography (LORETA), variable resolution emission tomography (VARETA), computer assisted tomography (CAT), EEG imaging or ultrasound scanning.

The term “objective symptom measurement”, as used herein, refers to any method that results in the collection of clinical data. These methods may include, but are not limited, to actigraph, Optax functionality testing and self-reporting questionnaires.

The term “multi-modality”, as used herein, refers to any collection of clinical data from at least two independent tests that results in a differential diagnosis of a disease or medical condition that either clinical test, alone, is unable to provide. Preferable combined methodologies may include, but are not limited to, combinations of electroencephalogram (EEG)/electrocardiogram (EKG), EEG/heart rate & blood pressure, EEG/biological indicators or EEG/cognitive indicators.

The term “platform”, as used herein, refers to any solid material configured to hold a plurality of pharmaceutical compounds.

The term “compartment”, as used herein, refers to any area on a platform wherein one pharmaceutical compound may be stored without risk of translocation relative to another pharmaceutical compound.

The term “aperture”, as used herein, refers to any configuration joining the platform and compartment such that a pharmaceutical formulation is dispensed.

The term “advancing mechanism”, as used herein, refers to any configuration moving the relative positions between the platform and compartment such that the next pharmaceutical formulation becomes aligned with the aperture.

The term “coding system”, as used herein, refers to any method that uniquely identifies a particular compartment.

The term “stabilizing”, as used herein, refers to the return of any neurotransmitter pathway activity to homeostasis. Specifically, the present invention contemplates neurotransmitter pathway stabilization to occur by, but not limited to, a pharmaceutical formulation comprising an anticonvulsant and a neuroactive modulator.

### BRIEF DESCRIPTION OF THE FIGURES

FIG. 1 is a perspective view of one possible embodiment of a pharmaceutical formulation dispensing device showing a notched skirt and tablet platform provided in a cut away view.

FIG. 2 displays one embodiment of a drug response probability flow chart.

FIG. 3 depicts exemplary data from a normative population database showing EEG responses to: i) antidepressants (Panel A) and ii) stimulants (Panel B).

FIG. 4 depicts an exemplary convalescent database. The X axis represents the numerical value of one multivariable score ascending from left-to-right. The Y axis represents the number of patients exhibiting any single multivariable score. The open squares plot a patient that are known not respond to a particular drug therapy (i.e., for example, an antidepressant). The crosshatched squares plot a patient that are known not to respond to a particular drug therapy (i.e., for example, an antidepressant).

FIG. 5 depicts exemplary data showing an averaged multivariate score calculated from approximately 30 multivariables collected from the same patient. The open circles represent an averaged multivariate score for patients not responding to a particular drug therapy (i.e., for example, antidepressants). The closed circles represent an averaged multivariate score for patients responding to a particular drug therapy (i.e., for example, antidepressants).

FIG. 6 depicts exemplary EEG data from patients exhibiting at least one symptom of an affective disorder: Squares: Theta wave from seven (7) patients responding to stimulants; Closed Circles: Alpha wave from thirty five (35) patients responding to antidepressants.

FIG. 7 depicts exemplary EEG data from patients exhibiting at least one symptom of an attentional disorder: Squares: Theta wave from fourteen (14) patients responding to stimulants; Closed Circles: Alpha wave from twenty five (25) patients responding to antidepressants.

### DETAILED DESCRIPTION

This invention relates to predicting the probability of a significant recovery following pharmaceutical treatment of nervous system disorders. In one embodiment, this invention relates to predicting the probability of a significant recovery from a nervous system disorder by a pharmaceutical formulation. In another embodiment, this invention relates to predicting the probability of a significant recovery following the treatment of nervous system disorders by at least one pharmaceutical formulation combined with a medical device. In another embodiment, this invention relates to predicting the probability of a significant recovery following the treatment of nervous system disorders by a formulation comprising an anticonvulsant and a neuroactive modulator.

### Nervous System Disorders

Psychiatric investigation is premised on the interaction of an individual human being with their environment. The psychological understanding of human behavior is provided by psychodynamic observation supplemented by knowledge derived from phenomenological and neurobiological research. Phenomenology and neurobiology are primarily concerned with detecting correlations between clinical syndromes (i.e., a set of exhibited symptoms) and pathological brain states. Current techniques of brain imaging is aimed at elucidating neurophysiological processes and may provide a basis to combine structural neuropathology with neuropathophysiology. Nemiah J. C., “The Varieties Of Human Experience” Br J Psychiatry, 154:459-66 (1989). The present invention contemplates evaluating observations derived from patient studies to generate a probability analysis reflecting underlying brain function to predict drug responsivity. It is also contemplated that these scores are predictive of an individual patient's prognosis (i.e., for example, the probability of having a significant recovery) when administered a specific pharmaceutical formulation. The present invention also contemplates statistically selected combination drug therapy that is effective for nervous system disorders, wherein sometimes the disorder is defined as either a psychiatric disorder or a neurological disorder.

The present invention contemplates general categories of psychiatric disorders to include, but not limited to, i) Disorders Usually First Diagnosed in Infancy, Childhood, or Adolescence; ii) Cognitive Disorders; Mental Disorders Due to a General Medical Condition; iii) Substance-Related Disorders; iv) Schizophrenia and Other Psychotic Disorders; v) Mood Disorders; vi) Anxiety Disorders; vii) Somatoform Disorders; Factitious Disorder; Dissociative Disorders; viii) Sexual and Gender Identity Disorders; ix) Eating Disorders; Sleep Disorders; x) Impulse-Control Disorders Not Elsewhere Classified; Adjustment Disorder; or xi) Personality Disorders. In one embodiment, a “psychiatric disorder” comprises a “neurobehavioral or intrapulmonary disorder”. In another embodiment, a “psychiatric disorder” comprises a “neurophysiological disorder”.

The present invention contemplates the general categories of neurological disorders to include, but are not limited to, i) convulsant disorders, ii) Parkinson's disease, iii) dyslexia, iv) migraine, v) pain and vi) stroke.

While it is not required to understand the exact mechanism of the present invention, it is believed that a combination therapy of an anticonvulsant and a neuroactive modulator stabilizes all chemical neurotransmitter pathways in a common fashion. For example, in the treatment of depression, a combination therapy comprising a pharmaceutical formulation comprising an anticonvulsant and a monoaminergic reuptake inhibitor is equally effective in stabilizing reduced activity in noradrenergic neurotransmitter pathways as in serotonergic neurotransmitter pathways. The primary neurotransmitter pathways (i.e., adrenergic, dopaminergic, serotonergic, cholinergic, glycinergic, glutaminergic, GABAergic etc.) are believed responsible for nervous system disorders and the present invention, therefore, contemplates a drug combination having therapeutic benefit on the majority of these disorders, regardless of their exhibition of differential symptomology.

I. Psychiatric Disorders

Antipsychotic drugs exert some beneficial effects in virtually all types of psychotic illness, and, contrary to common misconception, are not selective for schizophrenia. Moreover, antidepressant drugs that are especially beneficial in severe depression can also exert useful effects on less severe depressive syndromes and on conditions that are not obviously depressive in nature (i.e., panic attacks, bulimia nervosa, chronic pain, obsessive-compulsive disorder, and attention deficit-hyperactivity disorders). Also, many currently used antipsychotic drugs exhibit numerous and unpleasant side effects. Thus, in general, drugs presently used for nervous system disorders are not disease-specific but they do provide limited clinical benefit for specific syndromes or complexes of symptoms.

A. Disorders Usually First Diagnosed in Infancy, Childhood, or Adolescence

One advantage of the present invention contemplates the treatment of a patient (i.e., for example, a child) having a nervous system disorder including, but not limited to, mental retardation, learning disorders, motor skills disorder, communication disorders, pervasive developmental disorders, attention-deficit and disruptive behavior disorders, feeding and eating disorders, tic disorders, elimination disorders, other disorders of infancy and childhood or adolescence disorders. While treatment of all disorders within the above categories are contemplated by this invention, a non-limiting exemplary discussion of two specific embodiments appears below.

1. Tourette's Syndrome

Tourette's syndrome is a chronic nervous system disorder comprising vocal and motor tics, wherein an associated coprolalia affects only a minority of patients. Many children with Tourette's syndrome have associated obsessive-compulsive disorder (OCD) and/or attention deficit hyperactivity disorder (ADHD). Specific symptoms of Tourette's syndrome include, but are not limited to, uncontrolled head and neck movements, inappropriate language and excessively loud vocalizations.

Atypical antipsychotic drugs, clozapine, sulpiride, olanzapine, and risperidone, have been administered in an attempt to reduce tic's in Tourette's syndrome as well as the conventional antipsychotic used for Tourette's, pimozide. Risperidone is seen to be as effective as pimozide, with less side effects, including a much reduced risk of heart arrhythmia. Sindo et al., “Treatment Of Tics In Tourette Syndrome With Atypical Antipsychotic Drugs”, Ugeskr Laeger, 164(32):3755-9 (2002). However, no atypical antipsychotic is clearly effective for motor abnormalities in Tourette's syndrome.

2. Attention Deficit (Hyperactivity) Disorder

Attention deficit (Hyperactivity) disorder (ADD or ADHD) is usually first evident in childhood and is characterized by symptoms including, but not limited to, excessive motor activity, difficulty in sustaining attention, impulsiveness, academic difficulties (i.e., under achievement), impaired interpersonal relationships, or excitability.

It is believed that catecholamines (i.e., for example, adrenergic monoaminergic neurotransmitters) are involved in the control of attention at the level of the cerebral cortex. A variety of stimulant drugs (i.e., for example, dextroamphetamine, methylphenidate and the like) are known to improve ADD and ADHD. Some children, however, do not respond to these stimulant drugs, and their treatment is generally discontinued after a one month trial therapy. Adverse effects of stimulant drugs in children include insomnia, abdominal pain, loss of appetite, and weight loss. Alternatively, other drugs such as tricyclic antidepressants, antipsychotic agents and clonidine have all been administered with variable success.

B. Cognitive Disorders; Mental Disorders Due to a General Medical Condition

Another advantage of the present invention contemplates the treatment of patients for nervous system disorders including, but not limited to, deliria, dementias, amnestic disorders or mental disorders due to a general medical condition. While treatment of all disorders within the above categories are contemplated by this invention, a non-limiting exemplary discussion of one specific embodiment appears below.

1. Alzheimer's

An Alzheimer's patient usually develops symptoms comprising defects in cognitive abilities (i.e., for example, an impaired memory or thinking difficulties) and at least one second symptom including, but not limited to, aphasia (i.e., for example, problems using language), apraxia (i.e., for example, trouble carrying out motor activity, despite intact motor functioning), agnosia (i.e., for example, despite intact sensory functioning, the patient fails to recognize or identify objects presented) or impaired executive functioning (i.e., for example, problems abstracting, organizing, planning or sequencing information). These symptoms materially impair work or social functioning and result in a decline of mental functioning that begins gradually and worsens steadily.

Alzheimer's disease is the most common form of dementia. Four million Americans currently suffer from the condition, and experts estimate that 22 million people around the world will be so afflicted by 2025. Until recently, researchers had little understanding of the disorder's cause, and consequently preventive or curative therapies are presently lacking. Current research in the fields of epidemiology, genetics, molecular and cell biology, and other disciplines, however, are now identifying some of the underlying mechanisms.

For example, microscopic views of specific brain regions have revealed a loss of nerve cells in the hippocampus (i.e., comprising a memory center), and the cerebral cortex which controls cognitive processes such as, reasoning, memory, and language. Most of the degenerating nerve cells are cholinergic, and third treatment with acetylcholinesterase inhibitors (i.e., tacrine and donepezil) is known to slow the development of the early stages of Alzheimer's. This approach, however, does not prevent the eventual significant loss of cholinergic neurons.

Alternative therapeutic approaches are directed to designing compounds that block the ability of either the beta- or the gamma-secretase enzyme that produces amyloid peptide, or to alleviate this peptide's effects. Alternatively, antioxidants such as vitamin E or nonsteroidal anti-inflammatory drugs have potential to alleviate some of the toxic effects of amyloid deposits. For example, amyloid peptide accumulation may be reduced by Congo red or glycoaminoglycans by breaking down the aggregations of amyloid peptide from within. Also, vaccines made of β-amyloid peptide have potential to reduce the number of plaques.

Aside from the limited effectiveness of attempting to improve the fidelity of the cholinergic pathways in the early stages of Alzheimer's disease, there is currently no third or drug combination approach that has any impact on the stabilization or reversal of an Alzheimer's patient.

C. Substance-Related Disorders

Another advantage of the present invention contemplates the treatment of patients for nervous system disorders including, but not limited to substance dependence, substance withdrawal, substance abuse or substance intoxication. In one embodiment, the substance comprises alcohol, amphetamine or its derivatives (i.e., for example, methamphetamine), caffeine, cannabis, cocaine, hallucinogens, inhalants, nicotine, opioids, phencyclidine or sedatives. While treatment of all substance-induced disorders within the above categories are contemplated by this invention, a non-limiting exemplary discussion appears below.

Drugs that alter an individual's mood and feeling generally result in some form of a dependency upon taking that particular drug in the absence of any medical indications. More importantly, the drugs are not taken to “feel better”, the drugs are needed to “feel normal”. The intensity of this “need”, or dependence, may vary from a mild desire to a “craving” or “compulsion” to use the drug, and when the availability of the drug is uncertain, individuals may exhibit a preoccupation with its procurement (i.e., drug-seeking behavior).

The phenomenon of tolerance occurs following the administration of a wide variety of drugs. Not only the more popularly abused drugs such as alcohol, opioids, and hypnotics but others such as anticholinergics, dopaminerigic antagonists and tricyclic antidepressants. Tolerance and dependency, therefore, is a general phenomenon observed with many substances, and many independent biochemical and physiological mechanisms are involved.

The specific indications for treatment of chemical dependency vary with the specific drug, as well as social and cultural factors used in determining the particular pattern of drug use. In general, treatment is generally advisable when adverse consequences affect employment, family or other important social relationships or when a compulsive drug user voluntarily seeks help. Initially, most drug treatments are limited to the withdrawal process from the abused substance. The particular techniques and withdrawal medications are specific for each class of drug and personality of the dependent individual. Following a successful withdrawal period, continued behavioral or intrapulmonary modification and treatment of various psychiatric disorders (i.e., for example depression, anxiety or antisocial behaviors etc.) may be required to fully rehabilitate a chemically dependent individual.

D. Schizophrenia and Other Psychotic Disorders

Another advantage of the present invention contemplates treatment of patients for nervous system disorders including, but not limited to, paranoia, disorganization, catatonia, undifferentiated behavior, residual behavior, schizophreniform disorder, schizoaffective disorder, delusional disorder, brief psychotic disorder, shared psychotic disorder, psychotic disorder due to a general medical condition or a substance-induced psychotic disorder. While treatment of all disorders within the above categories are contemplated by this invention, a non-limiting exemplary discussion of one specific embodiment appears below.

1. Schizophrenia

Schizophrenia effects approximately 1% of the world-wide population. The most prominent symptoms include, but are not limited to, delusions and/or hallucinations. Over the past decade, use of the atypical antipsychotic drugs clozapine, risperidone, ziprasidone, aripiprazole, olanzapine, and quetiapine are routinely used for the treatment of schizophrenia. Differences in efficacy and tolerability between existing atypical antipsychotic drugs require individualization of drug therapy for patients with schizophrenia or schizo-affective disorder. Specifically, an optimal drug choice depends on determining whether there are clinically important differences between these drugs, and new drugs such as, for example, ziprasidone.

Ziprasidone is an effective antipsychotic drug for both positive and negative symptoms of schizophrenia, and long-term use has been effective in preventing relapse. Ziprasidone has also been suggested to have a significant serotonergic effect thus indicating a potential usefulness in antidepressant or antianxiety/anxiolytic therapy. Although ziprasidone has been associated with a low incidence of many common side effects, it may cause transient hyperprolactinemia. Additionally, ziprasidone is more likely than other atypical antipsychotic drugs to increase the QTc interval (i.e., the EEG Q-T interval corrected for heart rate). For acute psychotic symptoms in patients with schizophrenia, schizoaffective disorder, or acute mania, ziprasidone is administered twice daily at a usual daily dose of 80 to 160 mg, whereas 40 mg/d may be an effective maintenance dose. Stimmel et al., “Ziprasidone: An Atypical Antipsychotic Drug For The Treatment Of Schizophrenia” Clin Ther, 24(1):21-37 (2002).

Clozapine is a commonly prescribed antipsychotic agent associated with adverse extrapyramidal side effects. A comparison of clozapine to other antipsychotic drugs for extrapyramidal side effect risk results in the following rank order: clozapine<quetiapine<olanzapine=ziprasidone. Overall the side effects of antipsychotics are very drug specific. For example, quetiapine is fairly well tolerated, olanzapine is not well tolerated, risperidone is poorly tolerated, and amisulpride and ziprasidone have not been well evaluated. With the exception of clozapine, and perhaps quetiapine, atypical antipsychotics have brought only a relative avoidance of extrapyramidal side effects, therefore, strongly encouraging continued searches for novel antipsychotic agents. Tarsy et al., “Effects Of Newer Antipsychotics On Extrapyramidal Function” CNS Drugs 16(l):23-45 (2002).

E. Mood Disorders

Another advantage of the present invention contemplates treatment of patients having nervous system disorders including, but not limited to, major depression, mania, hypomania, bipolar disorders, dysthymic disorders, cyclothymic disorders, mood disorders due to a general medical condition or a substance-induced mood disorder. While treatment of all disorders within the above categories are contemplated by this invention, a non-limiting exemplary discussion of three specific embodiments appear below.

1. Depression

Major depression is a common and disabling disorder with far-reaching social and economic implications. Unfortunately, major depression treatments by current antidepressants show a response rate of only 65-70%. A recent survey of those skilled in the art concluded that for severe depression, standard antidepressants (i.e., for example, bupropion, selective serotonin reuptake inhibitors (SSRIs) or venlafaxine) should be combined with lithium or divalproex. Specifically, those in the art have recommended that divalproex be given as a third drug during initial treatment phases and then combined with a second antidepressant. Sachs et al., “The Expert Consensus Guideline Series: Medication Treatment Of Bipolar Disorder 2000” Postgrad Med Apr; Spec No:1-104 (2000).

Depressive delusions comprise reoccurring symptoms related to feelings of guilt, anxiety, poverty or disease and may include paranoid delusions. These symptoms are consistent with a diagnosis of a condition such as, but not limited to, major depressive disorder, endogenous depression or melancholia. Likewise, preliminary states (i.e., hypochondriatic fears of guilt and poverty) are sufficient to provide a differential diagnosis away from an anxiety condition that is associated with neurotic depression or dysthymia. Delusion is a particularly serious form of depression requiring specific therapeutic procedures apart from conventional therapy of affective disorders. Tolle R., “Delusion In Depression” Nervenarzt, 69(11):956-60 (1998).

Non-remissive patients (i.e., refractory or having an insignificant response) remain a significant problem in the treatment of depression with current monotherapy regimens. For example, only 64% of patients refractory to nortriptyline responded when switched to another antidepressant. Flint et al., J Affect Disord 36:95-105 (1996). Currently, it is believed that approximately 30-35% of patients treated for depression are refractory to drug treatment.

A hit-or-miss strategy (i.e., trial and error) is currently used by most clinicians when treating a non-remissive SSRI patient. A recent survey of clinicians revealed that when encountering a non-remissive SSRI patient, 84% increased the SSRI dose, 10% combined the SSRI with another antidepressant and 7% chose an alternative third antidepressant. When the only alternative presented was choosing an alternative antidepressant, 52% chose a recently available antidepressant, 34% chose another SSRI, 10% chose a tricyclic antidepressant, 2% chose a noradrenergic/serotonergic neurotransmitter reuptake inhibitor, 1% chose a monoamine oxidase inhibitor, and 1% chose an undefined “other” antidepressant. Of the clinicians choosing to combine the SSRI with another antidepressant, 30% chose bupropion and 22% chose lithium. Mischoulon et al., “Strategies For Managing Depression Refractory To Selective Serotonin Reuptake Inhibitor Treatment: A Survey Of Clinicians” Can J Psychiatry 45(5):476-81 (2000). None of those skilled in the art considered combining an anticonvulsant with the SSRI or alternative antidepressant drug.

2. Antidepressant Drugs

The effective treatment of depression with traditional antidepressants (i.e., for example, tricyclic antidepressants or monoamine oxidase inhibitors) is routinely accompanied by significant side effects. These side effects are considered a result of anticholinergic, anti-α-adrenergic, anti-histaminic and quinidine-like interaction. The introduction of antidepressant drugs having a more targeted mechanism of action (i.e., for example, selective serotonin reuptake inhibitors, selective norepinephrine reuptake inhibitor, bupropion, venlafaxine or nefazodone) were expected to result in a reduction of these side effects. Despite these expectations, pharmacodynamic and pharmacokinetic studies demonstrate that the targeted antidepressants still exhibit significant side effects. Stoudemire A., “Expanding Psychopharmacologic Treatment Options For The Depressed Medical Patient” Psychosomatics, 36(2):S19-S26 (1995).

a. Selective Serotonin Reuptake Inhibitors

The selective serotonin reuptake inhibitors (SSRIs) are exemplified by citalopram, escitaloproam, fluoxetine, fluvoxamine, paroxetine and sertraline. The classic side effect symptoms of SSRIs include, but are not limited to, headache, nausea, and sexual dysfunction. Individual differences in side effect symptomology may distinguish fluoxetine (predominantly nervousness and restlessness), sertraline (predominantly diarrhea loose stools), and paroxetine (dry mouth). The SSRIs all inhibit certain cytochrome P450 isoenzymes involved in the metabolism of drugs (i.e., for example, tricyclic antidepressants) and, therefore, SSRIs increase plasma concentrations of concomitantly administered tricyclic antidepressants. Andrews et al., “Contemporary Management Of Depression” Am J Med 97(6A):24S-32S (1994).

Specifically, SSRIs vary widely in their qualitative and quantitative interaction with cytochrome P450 isozymes in the liver. The SSRIs inhibit cytochrome P450-2D6 and are listed here in order of decreasing potency: paroxetine>norfluoxetine>fluoxetine>sertraline>citalopram>fluvoxamine. Fluoxetine interferes with carbamazepine metabolism at the level of cytochrome P450-3A and would also be expected to inhibit drugs having a similar chemical structure (i.e., for example, oxcarbazepine, oxcarbazepine derivatives and metabolites thereof). Similarly, paroxetine is a substrate of cytochrome P450-2D6 and may have similar effects as fluoxetine. Baumann P., “Pharmacokinetic-Pharmacodynamic Relationship Of The Selective Serotonin Reuptake Inhibitors” Clin Pharmacokinet, 31(6):444-69 (1996).

SSRI's have been combined with other antidepressant drugs and some anticonvulsants. For example, patients refractory to fluoxetine have been given a combination of fluoxetine and lithium carbonate. Buspirone combined with either fluoxetine or citalopram may also improve the antidepressant response in patients initially refractory to a third SSRI. Appelberg et al., “Patients With Severe Depression May Benefit From Buspirone Augmentation Of Selective Serotonin Reuptake Inhibitors: Results From A Placebo-Controlled, Randomized, Double-Blind, Placebo Wash-In Study” J Clin Psychiatry, 62(6):448-452 (2001).

SSRI's have also been combined with antipsychotic drugs (e.g., olanzapine) to improve the antidepressant response in refractory patients. In one study, third fluoxetine or olanzapine treatment resulted in a minimal to modest clinical effect, whereas a significant improvement resulted when these two drugs were combined. Shelton et al., “A Novel Augmentation Strategy For Treating Resistant Major Depression” Am J Psychiatry 158:131-134 (2001).

SSRI's have also been combined with tricyclic antidepressants in refractory patients. However, adverse pharmacokinetic interactions (i.e., for example, increased tricyclic antidepressant plasma levels) and lack of significant clinical success argue against administering this combination. Taylor D., “Selective Serotonin Reuptake Inhibitors And Tricyclic Antidepressants In Combination. Interactions And Therapeutic Uses” Br J Psychiatry, 167:575-580 (1995). Tricyclic antidepressants are suggested for combination with norepinephrine reuptake inhibitors or atypical antipsychotic drugs. Shelton R. C., “Treatment Options For Refractory Depression” J Clin Psychiatry, 60 Suppl 4:57-61 (1999).

The present invention contemplates one embodiment for the treatment of a non-remissive SSRI patient with a novel and surprising combination of an anticonvulsant and an antidepressant drug (i.e., for example, a neurotransmitter reuptake inhibitor), wherein at least one symptom of depression is reduced.

b. Bupropion

Bupropion (i.e., m-chloro-α-(t-butylamino)propiophenone; marketed as WELLBUTRIN; WELLBUTRIN SR; and WELLBUTRIN XL) is highly hygroscopic and susceptible to decomposition. When formulated as a hydrochloride salt, bupropion is a water-soluble crystalline solid having a melting point of 233-234° C. In one embodiment, bupropion is compounded and formulated as a preparation that reduces degradation in order to prolong shelf-life.

Prevention of bupropion degradation may be achieved by incorporating stabilizers within the pharmaceutical formulation. Degradation stabilizers may be incorporated into bupropion formulations including, but not limited to, instant release tablets, sustained release tablets, suppositories, topical agents, oral or intrapulmonary liquids and capsules. Effective stabilizers for bupropion formulations include, but are not limited to, organic acids, organic bases, inorganic acids, carboxylic acids, dicarboxylic acids, fumaric acid, amino acid salts and sodium metabisulfite. Exemplary stabilized bupropion formulations are disclosed in Ruff et al., U.S. Pat. No. 5,731,000, Maitra et al., U.S. Pat. No. 5,968,553, Kulkarni et al., U.S. Pat. No. 6,242,496 and Han et al., U.S. Pat. No. 6,333,332, all of which are hereby incorporated by reference.

Mechanism of Action

Acid-free stabilizers are useful for pharmaceutical formulations of bupropion when reduced production costs are desired. Alternatively, increasing the size of the bupropion particles prior to tablet compounding increases stability. In one embodiment, the particle size may range between 75-900 microns in diameter. A variety of particle sized, coated and uncoated, bupropion hydrochloride acid-free stabilized formulations are disclosed in Chungi et al., U.S. Pat. No. 6,306,436 and is hereby incorporated by reference.

Bupropion is known as a monoaminergic reuptake inhibitor having antidepressant properties (i.e., for example, WELLBUTRIN XR: currently marketed as an instant release formulation). Mehta, “Meta Chloro Substituted-α-Butylamino-Propiophenones” U.S. Pat. No. 3,819,706; and Mehta, “Meta Chloro Or Fluoro Substituted Alpha-T-Butylaminopropriophenones In The Treatment Of Depression” U.S. Pat. No. 3,885,046 (both patents hereby incorporated by reference). The effectiveness of bupropion's antidepressant effect has been considered equivalent to paroxetine (an SSRI). Doraiswamy et al., “Quality Of Life In Geriatric Depression: A Comparison Of Remitters, Partial Responders, And Nonresponders” Am J Geriatr Psychiatry, 9(4):423-428 (2001). For example, in one case a third administration of bupropion successfully reversed a previously intractable depressed and suicidal patient. Katz S. E., “Bupropion Treatment Of Refractory Depression” J Clin Psychiatry, 7:51-52 (1987).

Bupropion is classified as an “atypical antidepressant” similar to nefazodone, trazodone and venlafaxine. While it is not required to know the exact mechanism by which an invention operates, it is believed that atypical antidepressants such as bupropion have multiple sites of action. As such, these atypical antidepressants are suggested to be an important alternative to refractory third SSRI treatment. Horst et al., “Mechanisms of Action And Clinical Characteristics Of Three Atypical Antidepressants: Venlafaxine, Nefazodone, Bupropion” J Affect Disord 51(3):237-254 (1998).

Bupropion is known in the art to be as effective as tricyclic antidepressants. One significant advantage of bupropion is the occurrence of fewer anticholinergic, orthostatic, and cardiac conductive side effects. The usual adult daily dose of bupropion hydrochloride is 300-750 mg given in three daily doses and is suggested as a proper alternative for patients refractory to traditional tricyclic antidepressant therapy. Bryant et al., “Review Of Bupropion” Clin Pharm, 2(6):525-537 (1983).

Mechanistically, bupropion differs both clinically and pharmacologically from either the tricyclic antidepressants or the monoamine oxidase inhibitors. Preskorn et al., “Evaluation Of Bupropion Hydrochloride: The First Of A New Class Of Atypical Antidepressants” Pharmacotherapy, 4(1):20-34 (1984). Initially, bupropion was proposed as a relatively dopamine-specific antidepressant. Goodnick P. J., “Pharmacokinetics Of Second Generation Antidepressants: Bupropion” Psychopharmacol Bull 27(4):513-519 (1991). Bupropion also appears to have an unusual, although not fully understood, noradrenergic link that may be related to an active metabolite of bupropion (i.e., for example, hydroxybupropion). Notably, none of bupropion's antidepressant activity has been associated with serotonergic activity. Ascher et al., “Bupropion: A Review Of Its Mechanism Of Antidepressant Activity” J Clin Psychiatry, 56(9):395-401 (1995). Recently characterized as a selective norepinephrine and dopamine reuptake inhibitor, bupropion is effective when co-administered with venlafaxine, clozapine, lithium, topiramate and sodium valproate. Erfurth et al., “Bupropion As Add-On Strategy In Difficult-To-Treat Bipolar Depressive Patients” Neuropsychobiology, 45 Suppl 1:33-36 (2002).

Bupropion therapy is associated with a risk of seizure development, which can be minimized by multiple daily doses. Andrews et al., “Contemporary Management Of Depression” Am J Med 97(6A):24S-32S (1994). Specifically, bupropion's seizure risk is due to a lowering of the epileptogenic potential and is not recommended for patients who are predisposed to seizures. James et al., “Bupropion: Overview And Prescribing Guidelines In Depression” South Med J 84(2):222-224 (1991). One would conclude, therefore, that the art teaches away from administering bupropion to an epileptic patient. One embodiment of the present invention, however, contemplates the administration of an anticonvulsant (i.e., oxcarbazepine) and a monoaminergic reuptake inhibitor (i.e., bupropion) to an epileptic patient exhibiting at least one symptom of a nervous system disorder such that at least one symptom of the nervous system disorder is reduced.

Pharmacokinetics

The pharmacokinetic profile of bupropion follows a first-order absorptive phase, having a biphasic elimination with a redistribution half-life of about one hour and an elimination half-life of 11-14 hours. Bupropion presents a wide tissue distribution and is extensively metabolized by oxidation and reduction reactions. The present invention contemplates a pharmaceutical formulation comprising bupropion and an anticonvulsant drug that has a significant advantage over other standard antidepressant combination therapies. Although it is not necessary to understand the mechanism of an invention, it is believed that bupropion does not have significant pharmacokinetic interactions with other known anticonvulsants. As identified above, some antidepressant combinations result in pharmacokinetic interactions that consequently generate adverse side effects. (i.e., for example, tricyclic antidepressants and SSRIs).

Chemically, bupropion hydrochloride is a trimethylated monocyclic phenylaminoketone antidepressant. Following oral or intrapulmonary administration, bupropion hydrochloride is rapidly and significantly absorbed. Bupropion metabolism involves the cytochrome P450 2B6 system, not the cytochrome P450 2D6 system. A potential pharmacokinetic interaction between bupropion and fluoxetine (an SSRI) is speculated to underlie delirium and seizures when the two drugs are coadministered. Other potential bupropion pharmacokinetic interactions involve carbamazepine, cimetidine, phenobarbital, and phenytoin, all known to produce changes in hepatic metabolizing enzymes. Rotzinger et al., “Metabolism Of Some “Second”-And “Fourth”-Generation Antidepressants: Iprindole, Viloxazine, Bupropion, Mianserin, Maprotiline, Trazodone, Nefazonone, and Venlafaxine” Cell Mol Neurobiol, 19(4):427-442 (1999).

Furthermore, numerous known metabolites of bupropion, in both racemic and optically pure enantiomers, also inhibit monoaminergic reuptake systems. The racemic mixture of hydroxybupropion is an effective inhibitor of both norepinephrine and dopamine uptake while the optically pure (S,S)-hydroxybupropion is an effective inhibitor of only norepinephrine uptake. Fang et al., U.S. Patent Application No. 2002/0052341 (Filed: Nov. 16, 2001). While not intending to limit the present invention, it is believed that the primary antidepressant effect of bupropion is by the inhibition of monoaminergic neurotransmitter reuptake systems, such as, but not limited to, dopamine and norepinephrine. On the other hand, bupropion is believed to have no effect on the serotonergic neurotransmitter reuptake system (i.e., bupropion is not an SSRI).

Combination Therapies

As discussed above, combinations of bupropion or tricyclic antidepressants and SSRIs have been administered to treat refractory depression. The rationale behind this combination therapy being that both the adrenergic and the serotonergic systems are stimulated simultaneously. Nelson J. C., “Augmentation Strategies With Serotonergic-Noradrenergic Combinations” J Clin Psychiatry, 59 Suppl 5:65-68 (1998).

A bupropion/venlafaxine combination successfully reversed a chronic and recurrent major depression that had proven refractory to the administration of several antidepressants. Fatemi et al., “Venlafaxine And Bupropion Combination Therapy In A Case Of Treatment-Resistant Depression” Ann Pharmacother 33(6):701-703 (1999).

A bupropion/paroxetine combination successfully treated patients experiencing ineffective or intolerable third courses of desipramine, paroxetine, fluoxetine or bupropion. In addition to alleviating the depressive symptoms, the bupropion/paroxetine combination also reduced the third side effects. Marshall et al., “Paroxetine/Bupropion Combination Treatment For Refractory Depression” J Clin Psychopharmacol, 16:80-81 (1996). This coadministration of bupropion/paroxetine was specifically motivated by Marshall's prior literature review identifying success of other bupropion/SSRI combinations (i.e., fluoxetine and sertraline). Unlike the surprising invention contemplated herein, this literature review did not suggest any combinations comprising bupropion and anticonvulsants.

A bupropion/tranylcypromine (a monoamine oxidase inhibitor) combination successfully reversed a previously intractable refractory depressive state after years of unsuccessful multi-drug combination regimens. Pierre et al., “Bupropion-Tranylcypromine Combination For Treatment-Refractory Depression” J Clin Psychiatry, 61:450-451 (2000).

Other known combinations of bupropion include: i) naloxone or naltrexone, Dante, U.S. Pat. No. 5,512,593, Dante, U.S. Pat. No. 5,817,665 and Dante, U.S. Pat. No. 6,034,091; ii) (R)-tofisopam, Landry et al., U.S. Pat. No. 6,080,736, iii) an NMDA-glycine site agonist, Tsai, U.S. Pat. No. 6,228,875 and Tsai, “Methods For Treating Neuropsychiatric Disorders” U.S. Patent Application No. 2002/0035145 (Filed: Apr. 13, 2001); iv) 5-methoxy-carbonylamino-N-acetyltryptamine, Oxenkrug, U.S. Pat. No. 6,239,162; and vi) 1-threo-methyphenidate, Midha et al., U.S. Pat. No. 6,395,752 (all patents hereby incorporated by reference).

Despite the many known combinations of bupropion for the treatment of depression, the inclusion of any anticonvulsant with bupropion is unknown in the current treatment of nervous system disorders.

3. Mania

Mania is characterized by symptoms of excessive elation, typically tinged with dysphoria or marked by irritability, severe insomnia, hyperactivity, uncontrollable speech and activity, and impaired judgement. Mania is normally treated with antipsychotic drugs (i.e., for example, haloperidol), lithium salts or certain anticonvulsants for longer-term prevention of recurrences.

The present invention contemplates one embodiment comprising a formulation comprising an anticonvulsant and a monoaminergic reuptake inhibitor (i.e., for example, oxcarbazepine and bupropion) such that at least one symptom of mania is reduced.

4. Bipolar Disorders

A bipolar syndrome is characterized by symptoms of an uncontrollable alternation between the states of depression and manic. The therapeutic strategy is similar to that of mania (supra).

The present invention contemplates one embodiment comprising a formulation comprising an anticonvulsant and a monoaminergic reuptake inhibitor (i.e., for example, oxcarbazepine and bupropion) at least one symptom of a bipolar disorder is reduced.

F. Anxiety Disorders

Another advantage of the present invention contemplates treatment of a patient having a nervous system disorder including, but not limited to, agoraphobia, panic attack, specific phobia, social phobia, obsessive-compulsive disorder, posttraumatic stress disorder, acute stress disorder, generalized anxiety disorder, anxiety disorder due to a general medical condition or a substance-induced anxiety disorder. While all disorders in the above categories are contemplated by the present invention an exemplary non-limiting discussion is presented below.

Anxiety is not only a primary symptom of many psychiatric disorders but is also an almost inevitable component of many medical and surgical situations. Indeed, it is a universal human emotion, closely allied with appropriate fear, and often serves as an important psychobiological adaptive function.

A most important clinical generalization is that anxiety is rather infrequently a “disease” in itself. Clinical anxiety is typically associated with the “psychoneurotic” disorders, and therefore, cannot be readily explained in biological or psychological terms. One hypothesis, however, suggests the involvement of overactivity of adrenergic systems in the central nervous system. Hoeh-Saric R., “Neurotransmitters In Anxiety” Arch. Gen. Psychiatry, 39:735-742 (1982); and Gorman et al., “Pharmacologic Provocation Of Panic Attacks” In: Psychopharmacology: The Third Generation of Progress, pp. 985-993, Eds. Meltzer, H., Raven Press, New York (1987).

In addition, symptoms of anxiety are commonly associated with depression, dysthymic disorder (i.e., neurotic depression), panic disorder, agoraphobia and other specific phobias, obsessive-compulsive disorder and many personality disorders. Sometimes, despite a significant evaluation of a patient (either with or without a primary diagnosis) it may be desirable to simultaneously treat the anxiety. In such situations, antianxiety medications are frequently and appropriately used. Hollister et al., “Benzodiazepines, Current Update” Psychosomatics, 21, Suppl:1-32 (1980); and Lader et al., “A Comparison Of Buspirone And Placebo In Relieving Benzodiazepine Withdrawal Symptoms” J. Clin. Psychopharmacol., 7:11-15 (1987).

Currently, the most useful antianxiety drugs are thought to be the benzodiazepines. The specific benzodiazepine chosen seems to make little difference in the clinical outcome. However, in patients with impaired hepatic function or in the elderly, oxazepam is currently favored over lorazepam and alprazolam but chlordiazepoxide or diazepam is extensively prescribed to children. Baldessarini R. J., “Drugs And The Treatment Of Psychiatric Disorders” In: Goodman and Gilman's The Pharmacological Basis Of Therapeutics, Eighth Edition, pp. 428-429, Eds: Gilman et al., Permagon Press, New York (1990).

Panic disorder and social phobia are among the most disabling of the anxiety disorders. The emotional cost to the patient is exceeded only by the economic costs to the community (i.e., reduced productivity, lost workdays, increased health care costs etc.). It is imperative, therefore, that the medical community focus on the accurate diagnosis and effective treatment of these potentially devastating conditions.

Pharmacologic treatments for panic disorder and social phobia having limited efficacy and significant side effects have been available since the early 1960s. The benzodiazepines are usually the drug of choice, but cognitive impairment, physiological dependence, drug abuse, and withdrawal phenomena warranted a continued search for newer agents with an improved safety profile. Specifically, the SSRIs or anticonvulsants are known effective treatments for the symptoms of panic disorder and generalized social phobia. However, it is not at all clear whether the SSRIs are effective in treating nongeneralized social phobia. Their side effect profiles still can cause significant discomfort. Anticonvulsants are now emerging as a very important group of drugs in the anxiety disorders, with gabapentin having been the most extensively studied in social phobia. Davidson et al., “Panic Disorder And Social Phobia: Current Treatments And New Strategies” Cleve Clin J Med, 65 Suppl 1:SI39-47 (1998).

The neurobiological functioning of patients exhibiting symptoms of social phobias is very much like that of asymptomatic individuals. In general, a comprehensive study of phobias is currently hampered by the following: i) a lack of any accepted theory to guide research and aid the interpretation of results; ii) current research comprises only static comparisons between subject groups; and iii) data analysis that is oblivious to great individual variations (i.e, appropriate statistical analysis protocols are not followed). Clearly, alternative approaches to study the neurobiology of social phobia are necessary. For example, continuous and situation-specific measurement where subjects are used as their own controls and neurobiological correlates of clinical improvement following psychotherapy would be beneficial. Dewar et al., “The Quest For Biological Correlates Of Social Phobia: An Interim Assessment” Acta Psychiatr Scand., 103(4):241-3 (2001).

Antidepressant medications are also effective in the treatment of social phobia. Monoamine oxidase inhibitors, however, are currently avoided due to dietary restrictions and a relatively high rate of adverse effects. Reversible inhibitors of monoamine oxidase have less side effects but are also less effective. Currently, the selective serotonin reuptake inhibitors (i.e., for example, paroxetine) are becoming popular for the treatment of generalized social phobia. Schneier F. R., “Treatment Of Social Phobia With Antidepressants” J Clin Psychiatry, 62 Suppl 1:43-49 (2001). Other drug classes that have been evaluated are the benzodiazepines and adrenergic beta-blockers (i.e., propranolol).

G. Somatoform Disorders; Factitious Disorder; Dissociative Disorders

Another advantage of the present invention contemplates the treatment of a patient having a nervous system disorder including, but not limited to, conversion disorder, somatization disorder, undifferentiated somatoform disorder, hypochondriasis, pain disorder, body dysmorphic disorder, factitious disorder, dissociative amnesia, dissociative fugue, dissociative identity disorder or depersonalization disorder. While all disorders in the above categories are contemplated by this invention an exemplary non-limiting discussion of one specific embodiment appears below.

1. Conversion Disorder

A conversion disorder comprises at least one symptom including, but not limited to, a sensory deficit or voluntary motor function deficit. In one embodiment, the deficit includes, but is not limited to, pain or sexual dysfunction. Generally, preceding emotional conflicts or other tension and/or stress initiate or worsen the symptoms such that conversion may comprise a psychological factor. The expression of symptoms are serious enough to warrant medical evaluation and usually impairs social, occupational or personal functioning.

H. Sexual and Gender Identity Disorders

Another advantage of the present invention contemplates the treatment of patients having a nervous system disorder including, but not limited to, hypoactive sexual desire disorder, sexual aversion disorder, female sexual arousal disorder, male erectile disorder, female orgasmic disorder, male orgasmic disorder, premature ejaculation, dyspareunia, vaginismus, sexual dysfunction due to a general medical condition, substance-induced sexual dysfunction, exhibitionism, fetishism, frotteurism, pedophilia, sexual masochism, sexual sadism, transvestic fetishism, voyeurism or gender identity disorder. While all disorders in the above categories are contemplated by the present invention an exemplary non-limiting discussion of four related embodiments appearing below.

1. Paraphilias

Paraphilia is defined as comprising four of the above sexual disorders: fetishism, pedophilia, sexual sadism, and voyeurism. Paraphilia and paraphilia-related disorders are known to be associated with other psychiatric disorders. In particular, these disorders include mood disorders, dysthymic disorder, major depression, anxiety disorders, social phobia, psychoactive substance abuse (i.e., for example, alcohol and cocaine). Attention deficit hyperactivity disorder (ADHD) is diagnosed in 35.8% of paraphiliacs thereby providing a statistically significantly association with sexual disorders. Kafka et al., “A DSM-IV Axis I Comorbidity Study Of Males (N=120) With Paraphilias And Paraphilia-Related Disorders” Sex Abuse, 14(4):349-66 (2002).

A combination of psychostimulants (i.e., for example, methylphenidate-SR) and a selective serotonin reuptake inhibitors (i.e., for example, fluoxetine) was assessed as a pharmacologic treatment for men with paraphilias and paraphilia-related disorders. All patients were assessed for mood disorders and attention-deficit/hyperactivity disorder (ADHD). While a third SSRI diminished paraphilia behavior the addition of methylphenidate-SR resulted in a significant additional improvement. Kafka et al., “Psychostimulant Augmentation During Treatment With Selective Serotonin Reuptake Inhibitors In Men With Paraphilias And Paraphilia-Related Disorders: A Case Series” J Clin Psychiatry, 61(9):664-70 (2000).

I. Eating Disorders; Sleep Disorders

Another advantage of the present invention contemplates the treatment of patients having a nervous system disorder including, but not limited to anorexia nervosa, bulimia nervosa, obesity, primary insomnia, primary hypersomnia, narcolepsy, breathing-related sleep disorder, circadian rhythm sleep disorder, nightmare disorder, sleep terror disorder, sleepwalking disorder, insomnia related to Axis I or Axis II disorder, hypersomnia related to Axis I or Axis II disorder, sleep disorder due to a general medical condition or substance-induced sleep disorder. While all disorders in the above categories are contemplated by the present invention an exemplary non-limiting discussion of three specific embodiments appear below.

1. Bulimia Nervosa

Bulimia nervosa is a common eating disorder, especially in adolescent women. Biological, psychological, and social factors are implicated in its onset and is important in determining a successful treatment. Diagnosis of the syndrome involves evaluation of symptoms regarding forced vomiting following eating, usually resulting from an obsessive desire for weight reduction. Screening tools, laboratory findings, and physical findings are helpful in making the diagnosis. Other nervous system disorders commonly associated with bulimia include, but are not limited to, affective disorders, addictive disorders, anxiety disorders, personality disorders, and anorexia nervosa.

The etiology of bulimia nervosa is complex and involves biological, psychological, social, and family factors. Treatment, therefore, is comprehensive, individualized, and multifaceted. While many patients respond well to a combination of an antidepressant and cognitive behavioral or intrapulmonary therapy many patients are non-remissive. Wells et al., “Bulimia Nervosa: An Update And Treatment Recommendations” Curr Opin Pediatr, 13(6):591-7 (2001).

Clinical trials using various antidepressants have been performed including: i) tricyclic antidepressants (i.e., for example, imipramine, desipramine and amitriptyline); ii) selective serotonin reuptake inhibitors (i.e., for example, fluoxetine); iii) monoamine oxidase inhibitors (i.e., for example, phenelzine, isocarboxazid and brofaromine); and iv) other classes of drugs (i.e., for example, mianserine, trazodone and bupropion) where all groups of drugs exhibited similar efficacy. Bacaltchuk et al., “Antidepressants Versus Placebo For People With Bulimia Nervosa” Cochrane Database Syst Rev, 4:CD003391 (2001) Neuroendocrine and neurotransmitter function is suspected to reflect treatment success of bulimia (and anorexia, infra) and tend to normalize after symptom remission. One possible exception, however, is the observation of elevated cerebrospinal fluid 5-hydroxyindoleacetic acid concentrations in recovering patients suggesting that serotonin activity is still elevated after symptom remission. Elevated serotonin activity is consistent with other related behaviors, such as obsessionality with symmetry and exactness, harm avoidance, perfectionism, and behavioral or intrapulmonary over-control. Serotonergic medications are known to suppress these symptoms independently of their antidepressant effects. Refractory SSRI treatment in ill bulimia subjects could be a consequence of an inadequate supply of nutrients, which is essential to normal serotonin synthesis and function. These data raise the possibility that a disturbance of serotonin activity may create a vulnerability for the expression of a cluster of symptoms that are common in bulimia nervosa and that nutritional factors may affect SSRI response in depression, obsessive-compulsive disorder, or other conditions characterized by disturbances in serotonergic pathways. Kaye et al., “Serotonin Neuronal Function And Selective Serotonin Reuptake Inhibitor Treatment In Anorexia And Bulimia Nervosa” Biol Psychiatry, 44(9):825-38 (1998).

Bupropion (i.e., for example, Wellbutrin XL) is contraindicated for bulimic patients because of increased risk of seizure. As such, it can be concluded that the art teaches away from formulations comprising bupropion and anticonvulsants as an effective therapeutic strategy to treat bulimic patients. The present invention contemplates the administration of a formulation comprising bupropion and a neuroactive modulator to a bulimic patient such that at least one symptom of bulimia is reduced.

2. Anorexia Nervosa

Anorexia nervosa is a disorder characterized by symptoms of abnormal eating behavior, inappropriate weight loss, and disturbances in attitudes and perceptions toward body weight and shape. Although progress has been made in the treatment of anorexia nervosa, a substantial portion of patients are non-remissive to most treatments.

Anorexia nervosa is a complex psychiatric disorder with significant morbidity and mortality. Despite the fact that anorexia nervosa is currently considered a nervous system disorder confined to a fat-phobic Western culture, it's recent identification in non-Western societies suggests anorexia nervosa can exist without an associated fear-of-fatness. Specifically, anorexia nervosa is regarded as a primary nervous system disorder having an organic basis that may, or may not, be associated with other nervous system disorders.

Multiple endocrine and metabolic bioadaptive changes occur after prolonged starvation, primarily conservation of energy and protein. The identification of these endocrine findings in patients with anorexia nervosa may be secondary to these bioadaptive mechanisms. However, anorexia nervosa differs from simple starvation in that both feeding-stimulatory (orexigenic) and feeding-inhibitory (anorexigenic) signalling is overactive, thus producing a “mixed” signal regarding the homeostatic balance between satiety and hunger. Therapeutic intervention using receptor antagonists are suggested to generate more successful and targeted psychopharmacological treatment for anorexia nervosa. Inui A., “Eating Behavior In Anorexia Nervosa—An Excess Of Both Orexigenic And Anorexigenic Signalling?” Mol Psychiatry, 6(6):620-4 (2001).

Fewer than twenty controlled clinical trials are currently known that evaluate the effectiveness of various types of psychotherapy in anorexia nervosa. Little empirical evidence is available, therefore, on which to base treatment decisions regarding any psychological treatments for anorexia nervosa. Those in the art conclude there is a desperate need for further research in this area. Kaplan A. S., “Psychological Treatments For Anorexia Nervosa: A Review Of Published Studies And Promising New Directions” Can J Psychiatry, 47(3):235-42 (2002).

Primary anorexia nervosa is commonly associated with obsessionality and compulsiveness involving disturbances in neurotransmitters, notably serotonin. Yaryura-Obias et al., “The Integration Of Primary Anorexia Nervosa And Obsessive-Compulsive Disorder” Eat Weight Disord, 6(4):174-80 (2001). Selective serotonin reuptake inhibitors (SSRIs) are not useful when anorexia nervosa subjects are malnourished or significantly below their ideal weight. Fluoxetine; however, will reduce relapse rates when given after weight restoration. Refractory SSRI treatment in ill anorexia nervosa subjects could be a consequence of an inadequate supply of nutrients, which is essential to normal serotonin synthesis and function. These data raise the possibility that a disturbance of serotonin activity may create a vulnerability for the expression of a cluster of symptoms that are common in anorexia nervosa and that nutritional factors may affect SSRI response in depression, obsessive-compulsive disorder, or other conditions characterized by disturbances in serotonergic pathways. Kaye et al., “Serotonin Neuronal Function And Selective Serotonin Reuptake Inhibitor Treatment In Anorexia And Bulimia Nervosa” Biol Psychiatry, 44(9):825-38 (1998).

Bupropion (i.e., Wellbutrin XL) is contraindicated for anorexia nervosa patients because of increased risk of seizure. As such, a combination of any bupropion formulation with any anticonvulsant represents a therapeutic strategy that is not consistent with the current skill in the art. Consequently, current research actively teaches away from using bupropion, in any formulation, either by itself or in combination with other drugs to treat anorexia nervosa patients. In one embodiment, the present invention contemplates administering a formulation comprising bupropion and a neuroactive modulator to a bulimic patient such that at least one symptom of bulimia nervosa is reduced.

3. Obesity

Overweight and obesity have reached epidemic proportions in the United States. More than 61 percent of Americans aged 20 years and older are overweight and one-fourth of American adults are obese (an estimated 97 million), putting them at serious risk for poor health (DHHS, 2001). Yet, trends show that obesity continues to increase at alarming rates in men and women in most population groups. Among children six to seventeen years old, there seems to be an “obesity” crisis. Since 1980, the number of overweight children has doubled, and the number of overweight adolescents has tripled. In addition to being a major health hazard, obesity is associated with approximately 300,000 deaths a year in this country. Montague M. C., “The Physiology Of Obesity” ABNF J 14(3):56-60 (2003).

Over the past decade, there has been a tremendous increase in the understanding of the molecular and neural mechanisms that control food intake and body weight. Molecular and neural substrates are known to control body weight homeostasis. Such mechanisms include, but are not limited to, behavioral or intrapulmonary, neuroendocrine, and autonomic regulatory regions of the central nervous system. Non-neural mechanims involve hormones such as leptin and ghrelin that interact with the central nervous system. Zigman et al., “Minireview: From Anorexia To Obesity—The Yin And Yang Of Body Weight Control” Endocrinology, 144(9):3749-3756 (2003).

Genetic and environmental influences are known to play important roles in the prevalence of obesity. Human genetics will continue to make an invaluable contribution to the study of human obesity by identifying critical molecular components of the human energy balance regulatory systems, pointing the way toward more targeted and effective therapies and assisting the prediction of individual responses to environmental manipulations. O'Rahilly et al., “Minireview: Human Obesity-Lessons From Monogenic Disorders” Endocrinology, 144(9):3757-3764 (2003).

Obesity assessment involves measurement of the body mass index, waist circumference, and the identification of other risk factors. Management should include diet and exercise. Selected patients can be offered pharmacotherapy, of which only sibutramine and orlistat are FDA-approved for long-term use. Bariatric surgery is the only option that provides sustained and significant weight loss and should be offered to the severely obese patients. Mina et al., “The Treatment Of Obesity” Mo Med. 100(3):248-255 (2003).

In one embodiment, the present invention contemplates predicting the probability that an individual patient will lose weight subsequent to the administration of a pharmaceutical formulation comprising an anticonvulsant and a neuroactive modulator. In one embodiment, the probability prediction is calculated using multivariate Z scores collected from measurements including, but not limited to, neuroelectrical data, biological indicator data, cognitive indicator data, genotype profile data and the like.

J. Impulse-Control Disorders not Elsewhere Classified; Adjustment Disorder

Another advantage of the present invention contemplates the treatment of a patient for a nervous system disorder including, but not limited to, intermittent explosive disorder, kleptomania, pyromania, pathological gambling, trichotillomania or adjustment disorder. While all disorders in the above categories are contemplated by the present invention an exemplary non-limiting discussion of one specific embodiment is presented below.

1. Intermittent Explosive Disorder

Intermittent explosive disorder comprises symptoms where on several occasions the patient loses control of aggressive impulses, leading to serious assault or property destruction. In one embodiment, the aggressive impulses are markedly out of proportion to the seriousness of any social or psychological stressors.

K. Personality Disorders.

Another advantage of the present invention contemplates the treatment of patients having a nervous system disorder including, but not limited to, paranoid, schizoid, schizotypal, antisocial, borderline, histrionic, narcissistic, avoidant, dependent or obsessive-compulsive. While treatment of all disorders in the above categories are contemplated by the present invention an exemplary non-limiting discussion is presented below.

Personality disorders comprise a lasting pattern of behavior and inner experience that markedly deviates from norms of the patient's culture. In one embodiment, a personality disorder comprises the pattern in at least two behavioral or intrapulmonary traits. In one embodiment, the behavioral or intrapulmonary trait includes, but is not limited to, affect (i.e., for example, appropriateness, intensity, lability and range of emotions), cognition (i.e., for example, how the patient perceives and interprets self, others and events), impulse control or interpersonal functioning. In one embodiment, the disorder comprises a fixed pattern and affects many personal and social situations. In one embodiment, the fixed pattern has a long duration and has roots in adolescence and/or young adulthood. These symptoms cause clinically important distress or impair work, social or personal functioning.

II. Neurological Disorders

A. Convulsant Disorders

The term “epilepsies” is a collective designation for a group of central nervous system disorders having in common the repeated occurrence of sudden and transitory episodes (i.e., seizures) of symptoms including, but not limited to, abnormal motor control (i.e., convulsions) having a sensory, autonomic or psychic origin. The convulsions are nearly always correlated with abnormal and excessive discharges displayed in concurrent EEG recordings. The anticonvulsant drugs were initially developed to control patients experiencing epilepsy-related symptoms.

1. Anticonvulsant Drugs

a. Oxcarbazepine

Oxcarbazepine is a new anticonvulsant drug with a chemical structure similar to carbamazepine. The primary active metabolite of oxcarbazepine is 10, 11-dihydro-10-OH-carbazepine (monohydroxy derivative, MHD). During oxcarbazepine monotherapy, the half-life of MHD ranges from 10 to 15 hours in human patients following oxcarbazepine dosages of between 300-1,800 mg/day. Leppik I. E., “Antiepileptic Drugs In Development: Prospects For The Near Future” Epilepsia, 35 Suppl 4:S29-40 (1994). The distribution of 10-OH-carbazepine between blood cell compartments indicates a low level of plasma protein binding occurs but the metabolite demonstrated a marked affinity for the red blood cell. Jung et al., “The Distribution Of 10-Hydroxy Carbazepine In Blood Compartments” Biopharm Drug Dispos 18(1):17-23 (1997). The mean non-protein bound MHD fraction is approximately 56.7+/−5.5% but is increased when oxcarbazepine is administered in combination with other anticonvulsants such as, valproic acid, phenobarbital, methsuximide, or sulthiame. May et al., “Fluctuations Of 10-Hydroxy-Carbazepine During The Day In Epileptic Patients” Acta Neurol Scand 93(6):393-7 (1996). Similarly, during co-administration of oxcarbazepine and vilooxazoine an 11% increase in the non-protein bound plasma MHD concentration resulted but the oxcarbazepine plasma concentration was unchanged. Pisani et al., “Effects Of The Antidepressant Drug Viloxazine On Oxcarbazepine And Its Hydroxylated Metabolites In Patients With Epilepsy” Acta Neurol Scand, 90(2):130-132 (1994).

A 600 mg oxcarbazepine dose is maximally absorbed into the bloodstream at approximately 8 hours and is stable for an additional 16 hours thereby showing a plasma half-life of approximately 19.3+/−6.2 hours. Kristensen et al., “Pharmacokinetics Of 10-OH-Carbazepine, The Main Metabolite Of The Antiepileptic Oxcarbazepine, From Serum And Saliva Concentrations” Acta Neurol Scand, 68(3):145-150 (1983).

Oxcarbazepine, unlike its parent compound (i.e., carbamazepine) is metabolized by reduction and may not induce hepatic monooxygenase enzymes. For example, markers of hepatic monooxygenase enzyme activity (i.e., antipyrine, urinary 6-beta-hydroxycortisol, sex hormone binding globulin, and circulating androgens) maintained stable plasma levels during a two week course of twice daily 300 mg oxcarbazepine. Larkin et al., “Lack Of Enzyme Induction With Oxcarbazepine (600 mg Daily) In Healthy Subjects” Br J Pharmacol, 31(1):65-71 (1991). One embodiment of the present invention contemplates the administration of a formulation comprising oxcarbazepine and bupropion in treating patients having substance disorders and known to self-administer hepatic monooxygenase enzyme inducing drugs (i.e., for example, alcohol, barbiturates, opiates or methaqualone).

Oxcarbazepine detection by gas chromatography/mass spectrometry requires a bis-trimethylsilyl derivative of the oxcarbazepine enol and MHB or a tris-trimethylsilyl derivative of carbazepine-10,11-trans-diol. Each assay uses carbazepine-10,11-cis-diol as an internal standard. Using 0.5 ml of plasma the detection limits are 0.1, 0.1 and 1.0 ng/ml for oxcarbazepine, MBH, and the 10,11 transdiol metabolite, respectively. Von Unruh et al., Biomed Environ Mass Spectrum, 13(12):651-656 (1986).

b. Carbamazepine

Carbamazepine is a primary drug of choice for epilepsy. In addition to anticonvulsant activity, carbamazepine has been known to improve manic-depressive patients, even those refractory to lithium carbonate. Similar to the hydantoins, carbamazepine exerts its pharmacological effect via the sodium channel. Acute overdose side effects include stupor or coma, hyperirritability, convulsions, and respiratory depression. Long-term carbamazepine therapy is more likely to result in side effects including drowsiness, vertigo, ataxia, diplopia, and blurred vision.

c. Phenytoin, Mephenytoin and Ethotoin

Phenytoin, mephenytoin and ethotoin are primary anticonvulsant drugs for all types of epilepsy except substance seizures. The unique stabilizing effect of phenytoin on generalized epilepsy results from two actions: i) a decreased membrane permeability to sodium during neuronal resting potentials; and ii) an inhibition of voltage-sensitive sodium channels during neuronal action potentials. The toxicity of phenytoin is dependent upon the route of administration. For example, a high dose intravenous administration may result in side effects such as cardiac arrhythmias, hypotension and central nervous system depression. Acute oral or intrapulmonary overdosage, and chronic toxicity, are reflected in symptoms generally attributable to having a cerebellar and vestibular origin, including behavioral or intrapulmonary changes, increased frequency of seizures, gastrointestinal symptoms, gingival hyperplasia, osteomalacia, and megaloblastic anemia.

d. Barbiturates

Most barbiturates have some anticonvulsant activity. However, the relative ratio between their anticonvulsant action and induction of hypnosis limits their clinical applicability (i.e., anticonvulsant activity is negatively correlated with hydrophobicity). Consequently, sedation is the most frequent undesired side effect of barbiturate therapy. Phenobarbital and mephobarbital are useful in treating generalized tonic-clonic and partial seizures. Conversely, a deoxybarbiturate (i.e., primidone) is an effective agent for all types of epilepsy except absence seizures. The most common side effects when using primidone include sedation, vertigo, dizziness, nausea, vomiting, ataxia, diplopia and nystagmus.

e. Benzodiazepines

Most benzodiazepines have anticonvulsant activity but only clonazepam and clorazepate are currently approved in the United States for long-term treatment. Nonetheless, it is known that nitrazepam is useful for infantile spasms and that diazepam has a well-defined role in the management of status epilepticus. Although it is not necessary to understand the mechanism(s) of an invention, it is believed that the benzodiazepines exert their anticonvulsant effect by binding to the gamma-aminobutyric acid (GABA) receptor, thus augmenting the generalized inhibitory effect of this neurotransmitter system on postsynaptic neurons. The toxic side effects of benzodiazepines are relatively few, with cardiovascular and respiratory depression occurring only after intravenous administration. The most common side effects associated with long term oral or intrapulmonary therapy is drowsiness, aplastic anemia and lethargy. Specifically, clonazepam has anti-convulsant activity in patients exhibiting a wide variety of seizure disorders, with the notable exception of generalized clonic-tonic seizures.

f. Ethosuximide

Ethosuximide is specifically designed for the treatment of absence seizures. The mechanism of action of ethosuximide is not understood but it is known that it does not act by either an inhibition of sodium channels or by postsynaptic enhancement of gamma-aminobutyric acid activity. Ethosuximide, and its derivatives, are known to result in side effects concerning the gastrointestinal tract, central nervous system effects (i.e., Parkinson-like symptoms and photophobia), dermatological reactions, nausea, decreased platelet function, thrombocytopenia, hepatic failure and various blood anemias.

g. Valproic Acid

Valproic acid is effective against a wide variety of seizures while exhibiting only minimal sedative and other central nervous system side effects. Current theories identify the mechanism of action of valproic acid to include both inhibition of sodium channels and enhancement of gamma-aminobutyric acid activity.

B. Parkinson's Disease

Parkinson's disease comprises symptoms of bradykinesia, muscular rigidity, resting tremor and abnormalities in posture and gait. These symptoms give rise to a number of functional disabilities, including an inability to walk, a mask-like facial expression, an impairment of speech and skilled acts such as writing and eating. Despite advances in the understanding of the pathophysiology and treatment, the cause of Parkinson's remains unknown. Nevertheless, current research and drug therapy regimens are premised on the basis that Parkinson's disease develops due to a reduced availability of dopamine, a predominant neurotransmitter in the basal ganglia (i.e., the nigrostrial dopaminergic system), wherein repletion of homeostatic dopamine levels restores motor functions.

1. Antiparkinsonian Drugs

a. Levodopa

Levodopa (L-3,4-dihydroxyphenylalanine) is the immediate precursor to dopamine and readily crosses the blood brain barrier. This therapy generally results in a 50% reduction in symptomology in 75% of the treated patients. Essentially all symptoms, with the exception of dementia and postural instability initially respond to levodopa. In addition, the resultant increase in central nervous system dopamine levels also improves associated mood disorders (i.e., for example, depression). Chronic levodopa third administration, however, does ultimately result in the development of serious side effects in a significant number of patients. Further, the majority of patients treated with levodopa commonly experience some initial side effects including nausea, vomiting or cardiac arrhythmias (especially in predisposed patients). The majority of patients on long-term therapy develop abnormal involuntary movements and psychiatric disturbances. The prevalence of these critical side effects requires careful levodopa administration in patients with coronary insufficiency, cardiac arrhythmias, occlusive cerebrovascular disease, affective disorders or other major psychoses.

Generally, concurrent administration of carbidopa (an aromatic L-amino acid decarboxylase inhibitor) alleviates some levodopa side effects by allowing the administration of a lower levodopa dosage. Specifically, the dose of levodopa may reduced as much as 75% and the side effects of nausea and vomiting are largely eliminated.

The use of levodopa has one significant drawback. Many patients become refractory to the beneficial effects of administration, thus requiring the administration of other drugs, such as dopamine receptor agonists.

2. Clozapine

The anticholinergic activity of clozapine may reduce parkinsonian tremor.

3. Apomorphine

Although this dopamine agonist has an efficacious response in most Parkinson patients, it is an emetic and its use is very limited.

4. Ergolines

Derivatives of the ergot alkaloids (i.e., for example, bromocriptine, lisuride and pergolide) are known to stimulate dopamine receptors in the CNS, cardiovascular system, pituitary-hypothalamic axis and the gastrointestinal tract. Although high doses are capable of relieving Parkinson symptoms equivalent to levodopa, usually the ergolines are administered concurrently with levodopa.

As with most dopaminergic drugs, ergoline-induced side effects comprise nausea, vomiting and postural hypotension. In addition, the ergolines (in particular bromocriptine) may cause a “first-dose phenomenon” manifested by sudden cardiovascular collapse. Linch et al., “Bromocriptine Induced Postural Hypotension In Acromegaly” Lancet, 1:320 (1978). Additionally, auditory and visual hallucinations, symptomatic hypotension and cutaneous livedo reticularis are more frequent with bromocriptine than with levodopa.

C. Dyslexia

Dyslexia comprises symptoms related to the prevention of rapid and automatic reading abilities (in spite of a normal intelligence), visual capability and auditory acuity. Functional neuroimaging, such as tomography, has shown microscopic deficits of activation in the micropolygyria localized in the perisylvian cortex. Electrophysiological methods also reveal other specific abnormalities. Demonet et al., “Developmental Dyslexia: Contribution Of Modem Neuropsychology” Rev Neurol (Paris), 157(8-9 Pt 1):847-53 (2001).

Dyslexia is not confined to impairments in reading and spelling. There also appears to be a general cerebellar impairment involving the ability to perform skills automatically. Specific behavioral or intrapulmonary and neuroimaging tests indicate that 80% of individuals presenting with dyslexia have some cerebellar impairment. Nicolson et al., “Developmental Dyslexia: The Cerebellar Deficit Hypothesis” Trends Neurosci, 24(9):508-11 (2001)

Dyslexia is generally considered genetic in origin but the underlying neurochemical mechanisms are still unknown. Neuroimaging studies of dyslexic individuals indicate a possible cerebral cortical abnormality that might occur during specific stages of prenatal maturation. In vivo imaging studies (i.e., PET and functional MRI) identified some subtle differences in brain symmetry and an impairment in the brain visual mechanism. Habib M., “The Neurological Basis Of Developmental Dyslexia: An Overview And Working Hypothesis” Brain 123(Pt 12):2373-99 (2000).

The treatment of dyslexia is generally focused on improving functional skills and not on drug therapy trials. However, one random and blind clinical study assessed the efficacy of piracetam (a memory-enhancing drug that has been reported to facilitate reading skill acquisition) versus a placebo in children. The children were subtyped as “dysphonetic” or “phonetic” on the basis of scores from tests of phonological sensitivity and phoneme-grapheme correspondence skills. Overall, the piracetam group did not improve any more than the placebo group in any aspect of reading. Ackerman et al., “A Trial Of Piracetam In Two Subgroups Of Students With Dyslexia Enrolled In Summer Tutoring” J Learn Disabil, 24(9):542-9 (1991). Similarly, in two double-blind crossover studies the antimotion sickness drug, meclizine, was also found ineffective in improving reading skills of dyslexic children. These results were also found when meclizine was administered in combination with methylphenidate (regularly used to control attention deficit hyperactivity disorder). Fagan et al., “The Failure Of Antimotion Sickness Medication To Improve Reading In Developmental Dyslexia: Results Of A Randomized Trial” J Dev Behav Pediatr 9(6):359-66 (1988).

D. Migraine

Serotonin is suspected of having a role in the genesis of migraine attacks. Unfortunately, the tryptaminergic agents (i.e., for example, methysergide) are largely ineffective in treating migraines. However, the administration of an adrenergic beta-blocker (i.e., for example, propranolol), when given as a prophylactically, reduces the frequency and intensity of migraine attacks in 70% of patients. Interestingly, the β-adrenergic blocking effect of propranolol is not the suspected mechanism of action.

Ergotamine remains an important agent for symptomatic relief of the pain of migraine, particularly in those patients for whom naproxen or other non-steroidal antiinflammatory drugs provide insignificant relief. The efficacy of intravenous administration is immediate and dramatic in the vast majority of cases, but pain relief following oral or intrapulmonary administration is slow (i.e., 5 hours). Unfortunately, in some cases no relief is obtained following oral or intrapulmonary administration. Ergotamines are contraindicated in patients presenting in sepsis and those having vascular, kidney and liver diseases.

Tricyclic antidepressants and monoamine oxidase inhibitors are minimally effective, having an efficacy equivalent to the methysergides. However, non-steroidal antiinflammatory drugs (i.e., for example, salicyclic acid, naproxen, ibuprofen, mefenamic acid, flufenamic acid and tolfenamic acid) are as effective as the ergot alkaloids for menstrual migraine, but their efficacy regarding classical migraines is inconsistent.

E. Pain

Trigeminal Neuralgia

Trigeminal neuralgia is a very peculiar disease exhibiting excruciating and is considered “idiopathic”. This pain, also known as “tic douloureux”, is paroxysmic, very severe and can be triggered by a light cutaneous stimulus on a very localized facial area. The current opinion now favors a “neurovascular conflict” theory of origin: an artery, most often a loop of the superior or anteroinferior cerebellar artery, contacts the trigeminal nerve root causing localized demyelination and ectopic triggering of neuronal discharges. Joffroy et al., “Trigeminal Neuralgia. Pathophysiology And Treatment” Acta Neurol Belg, 101(1):20-5 (2001).

Anticonvulsant drugs are considered the drug of choice for trigeminal neuralgia. Carbamazepine has demonstrated effectiveness as evidenced in several controlled trials. Other studies indicate that baclofen and lamotrigine are usually provided for a non-remissive patient. Other, uncontrolled reports indicate that phenytoin, clonazepam, sodium valproate, gabapentin, and lidocaine will also relieve trigeminal neuralgia. Those having skill in the art, however, conclude that controlled trials testing the effect of some of these drugs, new drugs, and drug combinations are needed. Sindrup et al., “Pharmacotherapy Of Trigeminal Neuralgia” Clin J Pain 18(l):22-7 (2002).

Trigeminal neuralgia that is refractory to carbamazepine therapy has been treated with oxcarbazepine and is well tolerated with no significant side effects with the exception of occasional hyponatremia. Zakrzewska et al., “Oxcarbazepine: A New Drug In The Management Of Intractable Trigeminal Neuralgia” J Neurol Neurosurg Psychiatry, 52(4):472-6 (1989). Hyponatremia may also occur in children and, as such, electrolyte levels should be monitored during oxcarbazepine therapy. Approximately 20% of the adult population develops hyponatremia but no correlation is found between serum blood levels of oxcarbazepine or 10-OH-carbazepine. Borusiak et al., “Hyponatremia Induced By Oxcarbazepine In Children” Epilepsy Res, 30:241-6 (1998).

Phantom Pain

Damage to somatosensible afferent nerve fibers in the peripheral or central nervous system may often express symptoms involving intractable pain, termed phantom pain (i.e., a form of neuropathic pain). Often, the pain cannot be satisfactorily treated with nonsteroidal anti-inflammatory drugs but some antidepressants (tricyclic antidepressants) are effective for more or less continuous pain, while some anticonvulsants (carbamazepine, oxcarbazepine, phenytoin, lamotrigine or gabapentin) are effective for paroxysmal pain. Other effective drugs for phantom pain are: gamma-butyric acid agonists (baclofen), opiates (morphine preparations with a regulated release; fentanyl patch), the N-methyl-D-aspartate receptor antagonist amantadine, transdermally administered clonidine and locally applied lidocaine. Weber W. E., “Pharmacotherapy For Neuropathic Pain Caused By Injury To The Afferent Nerve Fibers”, Ned Tijdschr Geneeskd. 145:813-817 (2001).

Central Neuropathic Pain

Central neuropathic pain is a symptom of central nervous system lesions and is difficult to treat. Although it is not necessary to understand the mechanism of an invention, it is believed that neuronal hyperexcitability in damaged areas of the central nervous system plays a major role. The effectiveness of some anticonvulsants (i.e., for example, phenytoin, benzodiazepines, valproate, carbamazepine, pinelamotrigine, gabapentin or topiramate) is believed to be mediated by an increased GABA-mediated inhibition thereby decreasing abnormal neuronal hyperexcitability. These anticonvulsant compounds are considered in the art as effective as the antidepressant amitriptyline. Finnerup et al., “Anticonvulsants In Central Pain” Expert Opin Pharmacother. 3:1411-1420 (2002).

F. Stroke

Stroke is the third leading cause of death in the United States and is the leading cause of long-term disability, accounting for an estimated $40 billion each year in health care costs and lost productivity. According to the American Heart Association approximately 500,000 strokes occur annually in both men and women. However, more than half of total stroke deaths occur in women.

Stroke results from a sudden-onset disturbance in brain activity resulting when blood supply to the brain is either compromised or altogether blocked. More commonly known as a cerebrovascular accident (CVA), stroke can be caused by events such as, but not limited to, arteriosclerotic disease, hypertension, embolism or hemorrhage. Symptoms of stroke include, but are not limited to, debilitating paralysis, coma, convulsions, amnesia, dizziness, unsteadiness, weakness, impaired speech and vision, as well as other sensory and motor deficits.

Breakthroughs in biochemistry and medicine have shown that the excitatory neurotransmitter glutamate may play a significant role in the development of ischemia-produced brain damage following an episode of stroke. A toxic cascade of glutamate may spread to all brain regions, resulting in the devastating and sometimes irreversible effects of stroke and a transient ischemic attack.

Stroke may be initiated by a thrombotic brain blood vessel that prevents oxygen and nutrition getting to neurons. Neurons starved of oxygen and glucose release excessive amounts of glutamate from their synaptic bulbs. Glutamate then binds to N-methyl-D-aspartate receptors (NMDA receptors) and triggers excessive influx of sodium and calcium ions, along with water, into the postsynaptic neurons. Neuronal swelling thus initiates neuronal toxicity and apoptotic death. Glutamate-poisoned neurons also release excessive amounts of glutamate prior to apoptosis and a cycle of cell death is propagated.

The present invention contemplates the treatment of stroke by various embodiments of the present invention. In one embodiment, the treatment comprises a pharmaceutical formulation comprising an anticonvulsant and a glutaminergic receptor agent.

G. Drug Side Effects

One seemingly unavoidable aspect of modern medicine involves the presence of side effects for most pharmaceutical formulations. The present invention contemplates that, in one embodiment, the presence of side effects may be predicted because of psychological involvement. It is known that patients are more likely to report side effects when they are specifically asked, as opposed to making a voluntary report. For example, 20%-30% of hepatitis C patients are known to complain about neuropsychological side effects to standard antiviral pharmaceuticals. However, if hepatitis C patients are asked if they have ever experienced neuropsychological side effects, up to 70% have an affirmative response.

Although it is not necessary to understand the mechanism of an invention, it is beleived that side effects are a result of the interaction of the pharmaceutical formulation at a biological site that is not relevant to the individual patient's prescribed therapy. Side effects are, however, a result of drug interaction with biological systems. In one embodiment, the present invention contenplates predicting the probability that a specific pharmaceutical formulation will result in certain side effects. In one embodiment, the probability of pharmaceutical formulation side effects are predicted by a QEEG analysis of neuroelectrical scores.

H. Cancer Chemotherapeutics

The widespread nature and swift growth of cancerous lesions require rapid and accurate diagnosis and drug treatment therapies. Presently, clinicians are forced to rely upon past experience or recommendations published in the scientific literature that summarize trial-and-error results.

In one embodiment, the present invention contemplates predicting the probability that a cancer will undergo remission subsequent to the administration of a pharmaceutical formulation comprising an anticonvulsant and a neuroactive modulator. In one embodiment, the probability prediction is calculated using multivariate Z scores collected from measurements including but not limited to, neuroelectrical data, biological indicator data, cognitive indicator data, genotype profile data and the like.

### Therapy Response Probabilities

The present invention contemplates comparing individual patient data to a normative population and/or a convalescent population to determine the statistical probability of a significant recovery when administered a particular formulation (i.e., using for example, probability scores, univariate Z scores, multivariate Z scores, raw data etc.). In one embodiment, a clinical evaluation of a patient having at least one symptom of a nervous system disorder is performed using data related to various fields of the medical arts including, but not limited to, electrophysiology, biochemistry, behavior, cognition and physiology. Specifically, these clinical data include, but are not limited to, quantitative electroencephalography (QEEG), psychometric test batteries, biological indicators, brain cognition indicators, genotype allelic profiles, neuroimaging, objective measurement testing or multi-modality analysis. In one embodiment, the probability of a significant recovery by an individual patient exhibiting at least one symptom of a nervous system disorder is classified into one of three categories: i) sensitive, ii) intermediate, and iii) resistive.

In one embodiment, the present invention contemplates a probabilistic evaluation of an individual patient exhibiting at least one symptom of a nervous system disorder will significantly respond to a formulation comprising an anticonvulsant and a neuroactive modulator.

Quantitative Electroencephalographic Clinical Data

The classification of nervous system disorders using direct objective clinical data of the brain, or its functioning, may include, but is not limited to, electroencephalography (EEG), quantitative electroencephalography (QEEG), magnetic resonance imaging (MRI), functional magnetic resonance imaging (fMRI), positron emission tomography (PET), single photon emission computed tomography (SPECT), low resolution emission tomography analyses (LORETA), variable resolution emission tomography analyses (VARETA), as well as any other method that directly measures brain function. Other methods of collecting useful information for the probabilistic success of drug therapy include, but are not limited to, questionnaires, psychometric test batteries, biological indicators, cognition indicators, genotype allelic variations, objective test measurements and integration of multi-modality data.

In each of the assessment techniques, discrete, quantitative, univariate and/or raw clinical data is collected. In one embodiment, the collected data is compatible with a subsequent multivariate analysis. In one embodiment, the multivariate analysis results in calculation of the probability of a significant recovery for any specific drug therapy. One skilled in the art will easily recognize that the description below, calculating a multivariate Z score using quantitative electroencephalography, is analogously applicable to any method of collecting quantitative clinical data.

In one embodiment, the present invention contemplates a prognosis evaluation using clinical data parameters derived using quantitative electroencephalography (QEEG). Suffin, S., “Method For Classifying And Treating Physiologic Brain Imbalances Using Quantitative EEG” WO 01/58351. The process is premised on observations that drug therapy is known to produce differential changes in the EEG waveform. These drug-induced EEG modifications allow the construction of general classifications differentiating the responses between a normative population (i.e., comprising individuals asymptomatic of a nervous system disorder) and a convalescent population (i.e., comprising individuals symptomatic of the nervous system disorder that responded to a drug therapy regimen).

At least two types of analysis are possible according to the present invention—Type One and Type Two Analysis. Type One Analysis provides that patients are drug-free. Type Two Analysis provides for patients who will not or cannot be drug-free or for further analysis of those taking prescription drugs. Drug status must preferably duplicate the general population as well as fulfill the definition of a baseline measurement (i.e.,having less than 1% residual of other medications). Patients are preferably free of drugs for at least five half-lives, preferably seven half-lives, and more preferably ten half-lives of the parent drug and its metabolites. It is understood to one skilled in the art that this consideration is integrated into all embodiments of the QEEG analysis.

In one embodiment, the present invention contemplates comparing approximately seventy-four individual patient QEEG multivariate Z scores with QEEG multivariate Z scores drawn from a normative population database. In one embodiment, at least one individual patient multivariate Z score is aberrant when compared to the normative population multivariate Z score. In one embodiment, the abberant individual patient multivariate Z score is compared to the convalescent population database such that the probability of a significant response to an effective pharmaceutical formulation is identified. In one embodiment, the abberant individual patient multivariate Z score is higher than random chance (i.e., for example, a background multivariate Z score). In another embodiment, the abberant individual patient multivariate Z score is lower than random chance. The application of multivariate analysis upon the QEEG univariate parameters provides an ability to classify an individual's patient's Z score within a probability response category reflecting the probability of a significant response (i.e, for example, sensitive, intermediate or resistive).

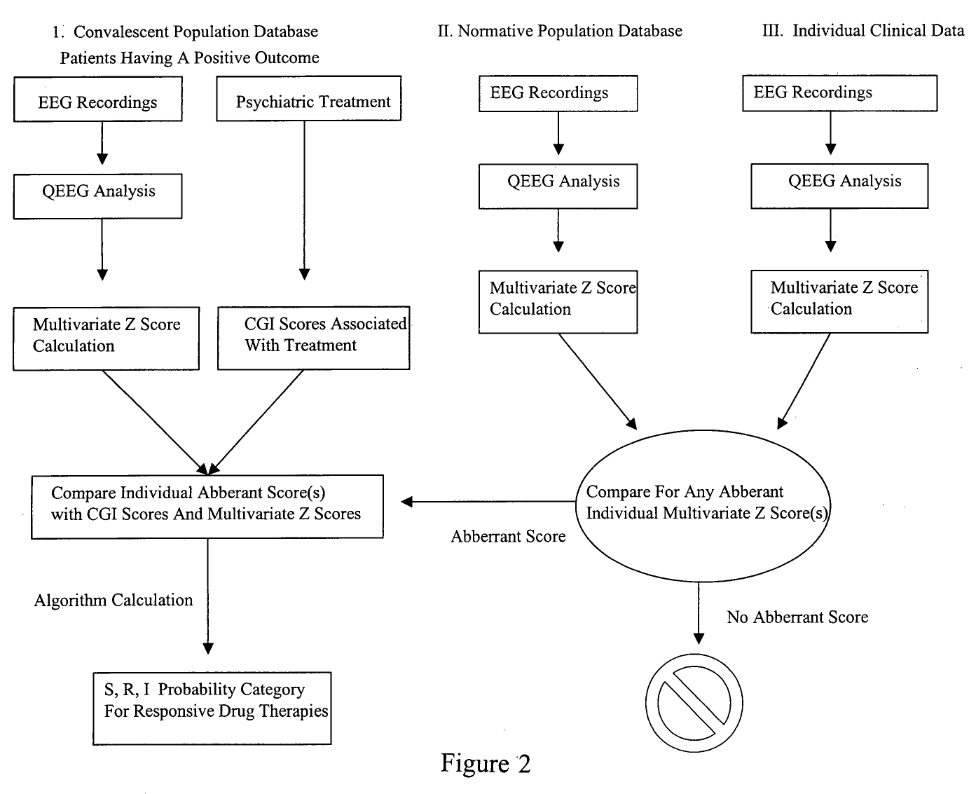

Multivariate Z score technology provides a simple and non-invasive approach to select the most optimal treatments to relieve symptoms of patients with nervous system disorders. A summary diagram depicting the comparative analysis flow between the convalescent population (I), the normative population (II) and an individual patient (III) is shown in FIG. 2. In all three databases, EEG is collected in digital form, wherein the EEG instrument records the voltage measured in the electrodes (calibrated in microvolts) as a function of time.

The convalescent population database (I) comprises clinical information of patients treated for variety of nervous system disorders with various pharmaceutical formulations collected over a period of years. In one embodiment, the convalescent population database comprises QEEG multivariate Z scores from patients exhibiting at least one symptom of a nervous system disorder.

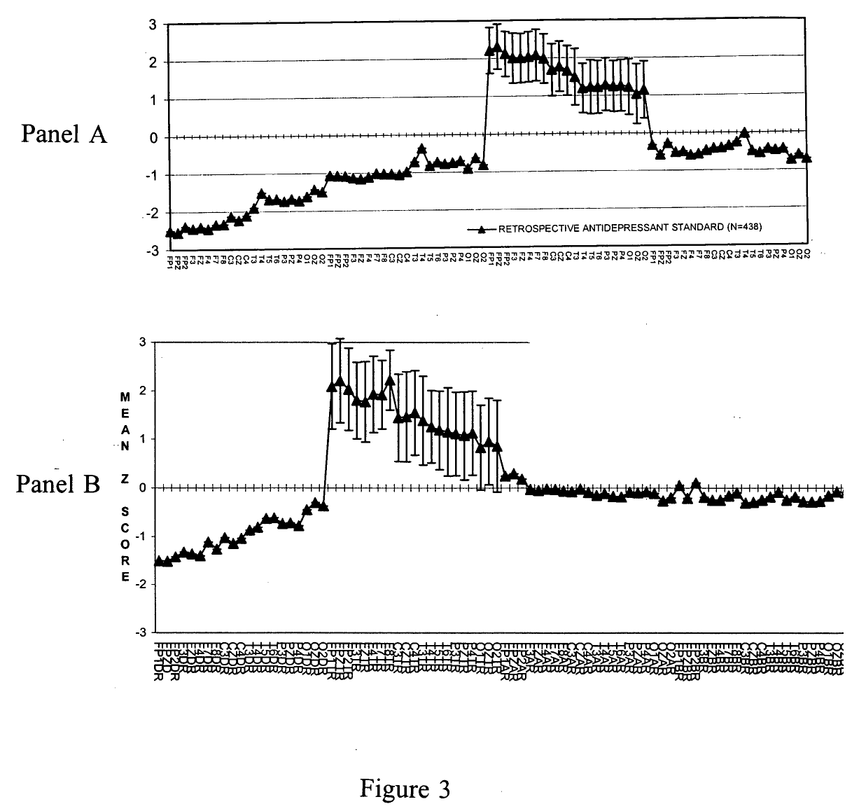

An exemplary QEEG analysis involves approximately 2400 univariables. In one embodiment, approximately 500 univariables are converted into approximately 74 multivariate Z scores (i.e., a multivariable). In one embodiment, at least one multivariate Z score comprises a single score having a value of ±2 or greater, wherein the score sufficiently identifies an abberant measurement. In one embodiment, a multivariate Z score represents the effect of a medication or a group of medications. In one embodiment, a multivariate Z score represents a specific anatomical brain area. In one embodiment, a factor analysis is employed to give greatest weight to those univariables that preserve the largest amount of total information of all the univariables in an anatomical group. In another embodiment, the univariables in an anatomical group are combined in a non-linear fashion to increase the separation of observed clusters within the EEG data. FIG. 3 depicts a QEEG pattern of patients responding to antidepressants or stimulants that illustrate this process.

FIG. 3 shows a convalescent population QEEG spectra for patients responding to either antidepressants (Panel A) or stimulants (Panel B). The x-axis represents the electrode sites of recording within four specific bandwidths (i.e., determined by the repeating sets of electrodes). The y-axis represents the mean univariate Z scores of the relative power spectrum (infra). The mean univariate Z score is a comparison of the individual patient's QEEG values to the normative database (i.e., a univariate Z score of 0 is the mean of the control group of asymptomatic individuals). Values further away from 0, either positive or negative, represent QEEG values different from values of asymptomatic control patients.

Panel A of FIG. 3 shows an exemplary group of 438 patients known responsive to antidepressants following a retrospective analysis. The QEEG measurement shown here is monopolar (i.e., single electrode) relative power. It should be noted that the data shows only 84 (i.e., 21 electrodes×4 frequency bandwidths) of the 2400 possible univariate Z scores available for analysis. The relative power (i.e., y-axis value) are different between the four bandwidths (i.e., the four repeating sets of electrodes). However, the relative power values of the mean univariate Z scores are fairly constant for each frequency bandwidth. This constant relative power within each frequency bandwidth allows this univariate Z score data to be simplified into multivariate Z scores. In one embodiment, two multivariate Z scores represent the statistical average of an entire individual bandwidth (i.e., one multivariate Z score representing the anterior portion of the head and a second multivariate Z score representing the posterior portion of the head). This calculation accurately demonstrates the clinical conclusion shown by the univariate Z scores in FIG. 3 Panel A that patients exhibiting at least one symptom of a nervous system disorder and responding favorably to antidepressants have a significantly elevated relative power spectrum within the third frequency bandwidth.

In FIG. 3 Panel B, 170 patients exhibiting at least one symptom of a nervous system disorder and responding favorably to stimulants have a significantly elevated relative power spectrum within the second frequency bandwidth.

Conversion of univariate Z scores to multivariate Z scores reduce the dimensionality of the data presented in FIG. 3 from 84 univariate Z scores to 8 multivariate Z scores while preserving the ability to quantitatively classify the clinical outcome. In one embodiment, a sensitive probability responder category comprises a frequency band multivariate Z score having a statistical significance above the 80th percentile, thereby making a significant recovery highly likely. In another embodiment, an intermediate probability responder category comprises a frequency band multivariate Z score having a statistical significance from between approximately the 20th percentile and 80th percentile, thereby making a significant recovery likely. In another embodiment, a resistive probability responder category comprises a frequency band multivariate Z score having a statistical significance below the 20th percentile, thereby making a significant recovery unlikely.

In one embodiment, the convalescent population database (I) comprises a patient's clinical outcome comprising a clinical global improvement (CGI) score. A CGI score represents a clinician's subjective assessment of the patient's response to administration of a pharmaceutical formulation. In one embodiment, the CGI scores comprises four values: i) CGI=0; when the patient presents with baseline symptomology (i.e., no response); ii) CGI=1; when the patient presents with a slight remission in at least one symptom of a nervous system disorder; iii) CGI=2; when the patient presents with a moderate remission in at least one symptom of a nervous system disorder; and iv) CGI=3; when the patient presents with a significant remission in at least one symptom of a nervous system disorder. Preferably, this subjective CGI rating system comprises values chosen by the same clinician for each individual patient. In one embodiment, the convalescent database (I) further comprises QEEG multivariate Z scores, that when correlated with the CGI scores, develop a mathematical model (i.e., for example, an algorithm) that allows the probabilistic determination of a significant recovery to a specific nervous system disorder subsequent administration of a specific drug formulations.

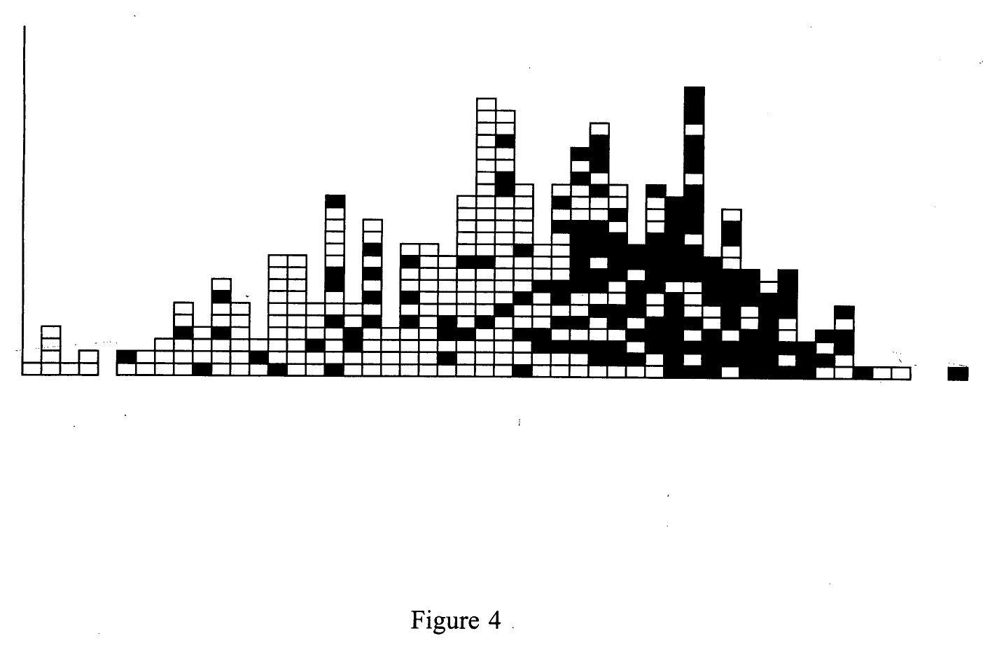

For example, the multivariate Z scores are correlated with prior patient response (i.e., measured by CGI score) to a particular medication by stratifying the patient response according to the distribution of univariate or multivariate Z scores. A stratified example of Z scores representing a single multivariable is shown in FIG. 4. The x-axis represents increasing values of a multivariable Z score being examined from left-to-right and the y-axis represents the number of patients exhibiting any specific multivariable Z score. The patients having a CGI of 2 or greater (i.e., termed known responders) are indicated by the cross-hatched squares. The patients having CGI of less than 2 (i.e., termed known non-responders) are indicated by the open squares. It is readily seen that patients known to respond to a particular drug therapy (i.e, for example, an antidepressant) have higher multivariable Z scores than those patients known not to respond.

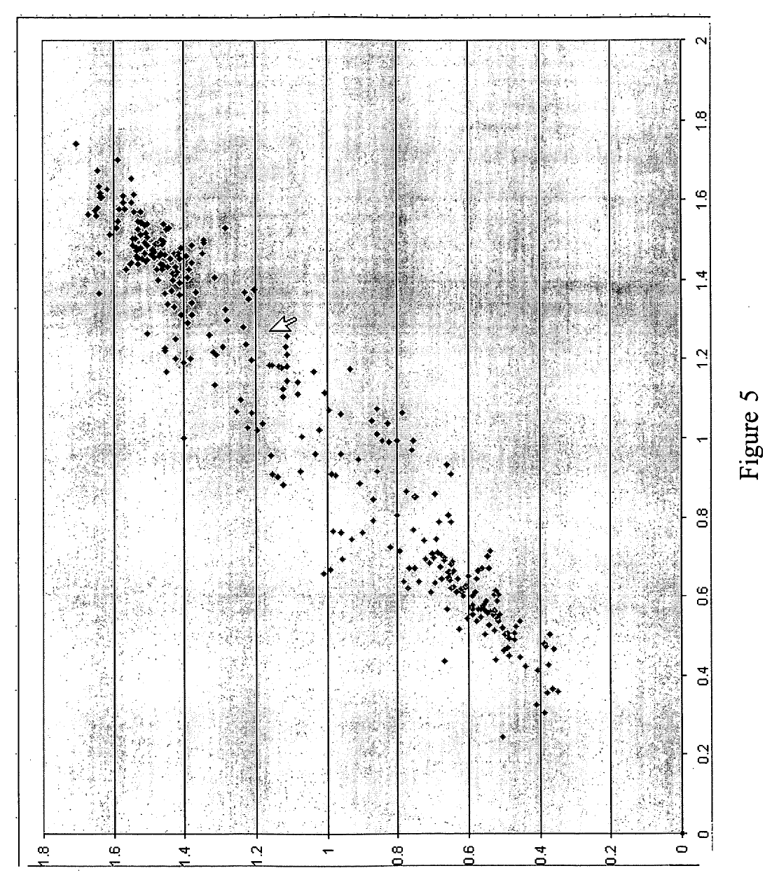

Many different multivariables are capable of providing response information for any particular drug therapy. In one embodiment, between approximately 20-30 different multivariables are averaged to provide a single multivariate Z score, wherein a larger score indicates a greater probability of a patient response to a drug. In FIG. 5, these averaged multivariate Z scores are plotted against the X-axis. In another embodiment, between approximately 20-30 multivariables are averaged to provide a single multivariate Z score, wherein a larger score indicates a greater probability of a patient not responding to a drug therapy other than the one under evaluation. In FIG. 5, these averaged multivariate Z scores are plotted against the Y-axis. FIG. 5 provides a clear distribution separation of non-responding patients versus responding patients to a particular drug therapy. For example, patients having a high probability of responding to a particular drug therapy (i.e., for example, an antidepressant) also have a high probability of not responding to any other drug therapy (i.e., for example, stimulants, antipsychotics etc.).

The normative population database (II) is internal to most neurometric analysis software systems. Alternatively, a normative EEG database is otherwise publicly available. However, the present invention contemplates a unique database comprising an augmented public domain database. The EEG measurements then are converted into the appropriate multivariate Z scores.

Individual patient data (III) is collected and processed in the same manner as the normative population database (II).

Prior to the comparison of individual patient scores with either a normative database or, subsequently a convalescent population database, tests of skewness and kurtosis are conducted on each of the multivariables to ensure that the original variable distribution is Gaussian.

Subsequently, an algorithm is constructed that provides a probability statement regarding whether the multivariable Z score for an individual patient measurement belongs to the distribution represented by a particular medication (i.e., for example, carbamazepine as shown in FIG. 4 Panel A) or belongs to the distribution defined by some other group (i.e., the rest of the population as shown in FIG. 4 Panel B). The probability is assessed by separately integrating seven ranges of the multivariate Z score distribution curve for all patients responding to drug therapy. The relative areas between these seven ranges of the multivariable Z score value establishes the probability that a particular value for an individual patient multivariable Z score will fall within one of the seven ranges by weighting the score for each drug formulation used to treat any particular nervous system disorder.

Calibration of this weighted score against actual patient records to determines what level of score actually translates into a specific probability of a significant response to a pharmaceutical formulation. In one embodiment, the probability of a significant response is classified as sensitive (S), wherein the probability ranges between approximately 80%-100%. In another embodiment, the probability of a significant response is classified as intermediate (I), wherein the probability ranges between approximately 20%-80%. In another embodiment, the probability of a significant response is classified as resistive (R), wherein the probability ranges between approximately 0%-20%.

A specific model algorithm is calibrated by performing a query (i.e., for example, making a comparison) to all patient responses that were not used in the construction of the algorithm. The query is divided into two subsets, the first is termed the tuning sample and the second is termed the final validation sample. The significantd algorithm is run using the tuning sample and the resulting distribution of scores is compared against known drug therapy responses. Thresholds for scores are then empirically set which implement the standards of S, I and R described above. The final validation sample utilizes these set thresholds for probability response classification. In order to preserve the fully prospective nature of this validation, no adjustment of the model parameters, including the S, I and R score thresholds, is made after this process. If the validation sample meets the specifications for predictive capacity, the model algorithm is then ready to be used to classify patients.

In one embodiment, EEG data is collected as univariate parameter data from electrodes placed at standard scalp locations (i.e., the International 10/20 System) on a patient who is awake and been unstimulated with eyes closed for at least twenty minutes. In another embodiment, artifact-free EEG data is collected for 180 seconds, preferably 200 seconds and more preferably 300 seconds.

The EEG data is digitized followed by Fast Fourier Transform (FFT) signal processing to yield a QEEG spectrum. This QEEG spectrum comprises thousands of electrical power measurements at various frequencies. The QEEG software then converts these power measurements into a multitude of derivative measures and values comprising both raw data and Z scores. In addition to identifying the power at each frequency averaged across the QEEG spectrum for each electrode, FFT signal processing of the raw EEG signal provides measurement and quantitation of other characteristics of brain electrical activity. This procedure results in the generation of approximately one thousand one hundred forty two (1142) scores comprising raw data scores and Z scores.

Although it is not necessary to understand the exact mechanism of an invention, it is believed that there is a relationship between various univariate EEG data parameters and brain activity. Exemplary univariate EEG data parameters include, but are not limited to, the following: i) “absolute power” is believed to be a measure of the strength of brain electrical activity; ii) “relative power” is believed to be a measure of how brain activity is distributed; iii) “symmetry” is believed to be a measure of the balance of the observed brain activity; iv) “coherence” is believed to be a measure of the coordination of the observed brain activity; and v) “frequency” is believed to be the average frequency of the electrical power within each of the major frequency bands (i.e., for example, delta, theta, alpha or beta). The present invention contemplates that these five EEG univariate measurements (i.e., for example, absolute power, relative power, symmetry, coherence and frequency etc.) are sufficient to establish the probability that a patient will, or will not, significantly respond to a pharmaceutical formulation.

Typically, QEEG univariate data parameters may be collected by, for example, a Spectrum 32 or EASY II (Cadwell Laboratories, Inc., Kennewick, Wash.) instrument. Readily available QEEG software then converts univariate EEG data into QEEG parameters (i.e., for example, NxLink). In one embodiment, a QEEG software package contains an age-defined normative databases comprising age regression expressions defining a distribution of features, wherein the features are functions of age. QEEG software extracts from the normative database an expected mean value and associated standard deviation for each feature from a subset within the normative population that is age-matched to an individual patient. QEEG software evaluates the difference between the value of each feature observed in the patient and the age-appropriate value predicted by the database age regression expressions.

QEEG software subsequently calculates a standard deviation (i.e., a univariate Z score) of the observed value of the patient from the age-corrected normative population. Currently available QEEG software is compatible with the collection of over 1000 univariate EEG data parameters from individuals ranging in age from 6 to 92 years.

Univariate EEG data parameters collected from a patient exhibiting at least one symptom of a nervous system disorder are extracted into an individual patient multivariate Z score by multivariate analysis techniques. Next, an individual patient multivariate Z score is compared to a similar multivariate Z score within a normative population. Although it is not necessary to understand the exact mechanism underlying an invention, it is believed that this comparison of an individual patient's multivariable deviations from the normative population mean value provides a precise system for recognition of a multitude of brain responses (i.e., for example, drug responsivity) that might be unrecognized when using only univariate signal analysis.

In one embodiment, the present invention contemplates special weighting functions assigned to specific univariate EEG data parameters prior to conversion into a QEEG multivariate Z score. For example, a weighting function allows the combination of univariate Z scores into an accurate multivariate Z score comprising measurements from different numbers and/or different positions of univariate electrodes (or pairs of univariate electrodes) by mathematically increasing or decreasing the signal strength to compensate for known, but uncontrollable, physical differences between the data collection points. This weighting process provides a normalization of the univariate Z scores such that the subsequent mathematical combination into the multivariate Z score accurately represents the actual electrophysiological data. In one embodiment, a mathematical combination of univariate Z scores comprise the calculation of the sum-of-squares for the univariate data points collected at each electrode pair given their appropriate weighting as described above. In one embodiment, the sum-of-squares for each univariate Z score is rounded to the nearest integer to create a multivariate Z-score. In one embodiment, the multivariate Z score is compared to a normative population database to determine if an abberant multivariate Z score is present.

In one advantage of the present invention, drug responsivity is predicted by a QEEG multivariate Z score. In one embodiment, the individual patient's QEEG multivariate Z scores are compared to a normative population database, wherein an abberant QEEG multivariate Z score is identified. In another embodiment, an individual patient's abberant QEEG multivariable Z score is compared directly with QEEG multivariable Z-scores within the convalescent population database to determine the probability of a significant response to a specific pharmaceutical formulation. Preferably, the comparison process comprises an evaluation of the statistical robustness of the individual patient's abberant multivariate Z score (i.e., by analyzing the number of standard deviations occurring within the univariate Z scores) to previously successfully treated patients to a specific pharmaceutical formulation. In one embodiment, an individual patient is classified as sensitive as predicted by a QEEG composite Z score, wherein the sensitive patient has a high probability of significantly responding to the identified pharmaceutical formulation.

In one embodiment, a resistive patient to one particular pharmaceutical formulation is compared to a sensitive patient to a third drug for any known nervous system disorder. In one embodiment, the resistive patient has in common at least one symptom of the sensitive patient. In another embodiment, the resistive patient has in common at least one multivariate Z score of the sensitive patient. In one embodiment, the resistive patient having a QEEG multivariate Z-score within the statistical norm of sensitive patients for the third drug is switched to the sensitive patient's drug formulation or drug combination having a high probability of a significant recovery.

As described above, the magnitude of the QEEG multivariate Z score is capable of establishing the probability of a significant drug response. Any particular QEEG parameter may ascertain a probabilistic response to a pharmaceutical formulation. For example, an absolute power average greater than 300 μV² in QEEG Parameter 1 predicts a response to antidepressants or α₂-adrenergic agonist drug classes. Similarly, a ratio of frontal to posterior EEG-alpha wave indices of less than 4 (e.g. QEEG Parameter 1) predicts a response to multiple drug classes. Many pharmaceutical formulations have been tested and tabulated. (See Table 4, WO 01/58351).

One embodiment of the present invention contemplates QEEG multivariate Z scores that identify individual patients that are sensitive, intermediate or resistive to pharmaceutical formulaitons comprising an anticonvulsant (i.e., for example, oxcarbazepine) and a monoaminergic reuptake inhibitor (i.e., for example, bupropion).

Psychometric Test Batteries

Cognitive deficits may be analyzed by psychometric test batteries using a resultant calculated multivariate Z score using the raw univariate data. Refractory patients to fluoxetine (an SSRI) are known to perform significantly worse in aspects related to executive functioning than patients who are not refractory to fluoxetine. Prefrontal dysfunction in subjects with major depression, therefore, may be predictive of poor response with particular medications. A pretreatment assessment of a patient's executive functions may play a particular role in the prediction of patients likely refractory to fluoxetine. Dunkin et al., “Executive Dysfunction Predicts Nonresponse To Fluoxetine In Major Depression” J Affect Disord, 60(1):13-23 (2000).

Biological Indicators

Numerous endocrine abnormalities are found in depressive illness. These abnormalities are known as useful markers in the diagnosis, prediction of treatment response, monitoring treatment outcome and understanding depression etiologies. Measurements of these endocrine biomarkers may comprise univariate data and are thus compatible to calculate multivariate Z scores (supra).

Five primary endocrine systems (i.e., hypothalamic-pituitary-adrenal axis, hypothalamic-pituitary-thyroid axis, growth hormone regulation, prolactin regulation and pineal function) all respond to clinical depression with an altered activity that provides biological indicators relevant to the probabilistic success of the administration of a pharmaceutical formulation. For example, the traditional dexamethasone suppression test (DST) is affected by a variety of diseases and pathophysiological conditions. Observed variability in dexamethasone bioavailability, however, argues for more refined, or alternative tests, of hypothalamic-pituitary-adrenal function to provide more reliable data for drug response prediction.

A low nocturnal output of melatonin (produced by the pineal gland) is a known biological indicator to diagnose unipolar and bipolar affective disorder. Similarly, seasonal affective disorder, another form of depression, is influenced by phase delays in the melatonin rhythm. Other hormonal abnormalities in depression are also known to be reflected in pituitary hormone release. Brown G. M., “Neuroendocrine Probes As Biological Markers Of Affective Disorders: New Directions” Can J Psychiatry, 34:819-23 (1989).

Major depressive disorders may be identified by a blunted prolactin response to D,L-fenfluramine administration. Fluoxetine-induced antidepressant responses are negatively correlated with fenfluramine-induced prolactin release. These observations suggest that a low baseline serotonin activity may be associated with refractory fluoxetine treatment of depression. New et al., “Serotonin And The Prediction Of Response Time To Fluoxetine In Patients With Mild Depression” Psychiatry Res, 88(2):89-93 (1999). One embodiment of the present invention contemplates an endocrine hormone plasma pattern that identifies a SSRI-refractory patient that has a high probability of responding to a formulation comprising an anticonvulsant and a neuroactive modulator. The present invention also contemplates an embodiment where an endocrine hormone plasma pattern identifies a depressed patient that has a high probability of reducing at least one symptom by the administration of a pharmaceutical formulation comrpising oxcarbazepine and bupropion.

Brain Cognitive Indicators

The cognitive functioning of the brain is dependent upon the interaction between various neurochemical pathways. Most of the cognitive pathways involve enzymes that slightly modify the chemical structure of either a drug or a naturally occurring compound (i.e., for example, a protein, hormone or neuroactive modulator). Although it is not necessary to understand the mechanism of an invention, it is believed that the rate of these pathways reflect the brain's cognitive ability. Further, it is assumed that as the rate of these pathways are reduced, the brain's cognitive ability is, of consequence, also reduced.

Brain glucose utilization rates can easily be measured and converted into multivariate Z scores. Brain glucose utilization alterations are known to be associated with the refractory response of fluoxetine treatment of depressed patients. Evaluations in glucose utilization in several brain regions demonstrated response-specific brain region patterning during the first six weeks of SSRI therapy that provides a basis to identify refractory patients. Specifically, following Week 1 of fluoxetine treatment, positron emission tomography (PET) showed similar brain glucose utilization patterns between patients responding to the SSRI and patients refractory to the SSRI. After six weeks of SSRI treatment, however, the responding patients had decreased glucose utilization in the limbic and striatal areas in conjunction with increased glucose utilization in the brain stem and dorsal cortical areas. The patients refractory to six weeks of SSRI treatment, however, had glucose utilization patterns similar to that observed following the first week of treatment. Specifically, these refractory patients did not have either a decreased glucose utilization in the subgenual cingulate or an increase in prefrontal glucose utilization. Mayberg et al., “Regional Metabolic Effects Of Fluoxetine In Major Depression: Serial Changes And Relationship To Clinical Response” Biol Psychiatry 48:830-843 (2000). One embodiment of the present invention contemplates a brain glucose utilization pattern that identifies SSRI-refractory patients having a high probability of responding to a formulation comprising an anticonvulsant and a neuroactive modulator. In another embodiment, the present invention contemplates a brain glucose utilization pattern that identifies a depressed patient having a high probability of reducing at least one symptom by the administration of a formulation comprising oxcarbazepine and bupropion.

In one embodiment, brain cognitive pathways may be measured by using radiolabeled medicines or drugs. In one embodiment, these labels may be visualized using various scanning techniques known in the art. In another embodiment, tagged compounds (either radiolabeled or not radiolabeled) may also accumulate at a specific step in the enzyme pathway because the compound has become an incompatible substrate for the next enzyme. Measuring the rate of accumulation of the tagged compound is a reliable method of assessing the rate of a specific enzyme system.

Genotype Allelic Variants

Genotype allelic variants provide discrete quantitative information that may be analyzed by multivariate Z scores. Genotype allelic variants provide probabilistic information relative to the refractory treatment of depression. A patient response to paroxetine (an SSRI) demonstrates a classic single-gene mendelian distribution of functional serotonin reuptake transporter polymorphisms. The serotonin reuptake transporter proteins are expressed in two polymorphic forms: a long variant and a short variant. When a patient expresses either the homozygous long genotype or the heterozygous long/short genotype antidepressant responses are not significantly different. When the patient expresses the homozygous short genotype, however, the antidepressant effect of paroxetine is significantly different from both the homozygous long genotype and the heterozygous long/short genotype. Zanardi et al., “Efficacy Of Paroxetine In Depression Is Influenced By A Functional Polymorphism Within The Promoter Of The Serotonin Transporter Gene” J Clin Psychopharmacol 20:105-107 (2000). One embodiment of the present invention contemplates a genotype profile that identifies non-remissive SSRI patients having a high probability of responding to a formulation comprising an anticonvulsant and a neuroactive modulator. In another embodiment, the present invention contemplates a genotype profile that identifies a depressed patient having a high probability of reducing at least one symptom by the administration of a formulation comprising oxcarbazepine and bupropion.

In one embodiment, single nucleotide polymorphisms (i.e., SNPs) are contemplated by the present invention to provide a quantitative score on which to generate multivariate Z scores. In one embodiment, the SNP comprises an altered protein conformation that results in an altered enzyme activity. In one embodiment, the resultant alteration in enzyme activity results in a nervous system disorder.

Neuroimaging

Digitization of neuroimages provides a multitude of clinical data that is compatible for calculation into multivariate Z scores. Neuroimaging studies are categorized as: i) structural; exemplified by computed tomography (CT), magnetic resonance imaging (MRI), low resolution emission tomography analyses (LORETA); and ii) functional; exemplified by positron emission tomography (PET), functional magnetic resonance imaging (fMRI), single photon emission tomography (SPET).

Advances in physics, computing, and signal processing have provided a range of computerized brain imaging technologies that facilitate examination of the brain as a dynamic system. These recent advances in brain imaging advances has had a direct application in the practice of neuropsychiatry.

Specifically, the field of neuroimaging has made several recent advances understanding Alzheimer's disease. Early detection, monitoring cognitive and pathological progression, and response to clinical intervention has been evaluated by PET, fMRI, and structural MRI. Burggren et al., “Structural And Functional Neuroimaging In Alzheimer's Disease: An Update” Curr Top Med Chem, 2(4):385-93 (2002).

Functional brain imaging studies of nervous system disorders, such as major depression, have consistently revealed hypometabolism or hypoperfusion in specific regions of the prefrontal cortex and basal ganglia. Studies of cognitive functioning in major depression have suggested that some but not all subjects exhibit cognitive deficits that are consistent with frontal-subcortical dysfunction.

Objective Symptom Measurements

Objective symptom measurements result in the collection and compilation of discrete univariate clinical data. These data, therefore, may be subjected to statistical analysis and calculation of multivariate Z scores.

Diagnostic criteria using objective symptom measurements have been constructed for eating disorders (ED) (i.e, anorexia nervosa, bulimia nervosa and non-specified eating disorders). Specifically, these data include that collected from sleep polysomnography, actigraph studies and self-report questionnaires. Golan et. al., “Sleeping And Eating Disorders” Harefuah, 141(6):552-9, 577 (2002).

Actigraph evaluation was also used to study pharmacodynamic effects of methylphenidate in ADHD children. Specifically, measures of drug efficacy were obtained from a Motionlogger actigraph to quantify activity and from the Swanson, Kotkin, Agler, M-Flynn, and Pelham (SKAMP) rating scale to quantify two domains of behavior (attention and deportment). This measure was able to detect significant reductions in activity and inappropriate behavior in the classroom. Swanson et al., “Efficacy Of A New Pattern Of Delivery Of Methylphenidate For The Treatment Of ADHD: Effects On Activity Level In The Classroom And On the Playground” J Am Acad Child Adolesc Psychiatry, 41(11):1306-14 (2002).

Multi-Modality

Multi-modality comprises the integration of two or more independent clinical tests, each of which comprise discrete and independent clinical data. As such, a unique database may be compiled that results in multivariate Z scores of these integrated data.

QEEG analysis may be combined with regional blood flow neuroimaging that is associated with therapeutic responses to antidepressant therapy. One specific QEEG parameter, cordance, is correlated with regional cortical perfusion, and has predicted the clinical response of patients having major depression. Specifically, following a 48 hour treatment with any one of a variety of antidepressants, patients responding to the therapy had decreased prefrontal QEEG parameters, whereas patients that were refractory to the antidepressant treatment did not have the decreased prefrontal QEEG parameter. The prefrontal region may, therefore, play a role in mediating response to medications with different mechanisms of action.

Increasingly, diagnostic images are being acquired from the same patient using two or more diagnostic imaging modalities. An MRI image will show essentially anatomical information. A SPECT image, using HMPAO, will show the cerebral perfusion of the same area(s). The ability to overlay such anatomical and functional data is an important tool in radiology. Preliminary observations have evaluated comprehensibility, information loss and efficiency in conveying all available MRI-SPECT imaging information simultaneously. Condon B. R., “Multi-Modality Image Combination: Five Techniques For Simultaneous MR-SPECT Display” Comput Med Imaging Graph, 15(5):311-8 (1991)

Distinguishing epileptic events from non-epileptic paroxysmal neurologic events represents a common diagnostic challenge. For example, syncope can appear similar to atonic and convulsive seizures. Similarly, epileptic seizures may resemble breath holding and benign paroxysmal vertigo, classic migraine, transient global amnesia, transient ischemic attacks, and sleep disorders, including nocturnal movements, parasomnias, or narcolepsy. A correct diagnosis can be established and appropriate treatment instituted by routine and prolonged EEG and EKG that is optionally combined with appropriate sleep studies. Morrell M. J., “Differential Diagnosis Of Seizures” Neurol Clin 11(4):737-54 (1993).

EKG/EEG recordings were compared between 67 epileptic seizures and 38 psychogenic non-epileptic seizures. The ictal heart rate was higher during and after epileptic seizures for both convulsive and non-convulsive spells. However, a concurrent quiet staring spell differentiated the convulsive spell from the non-convulsive spell with a positive predictive value of 97%. An increase in ictal heart rate, therefore, during a concurrent quiet staring spell can distinguish between convulsions having an epileptic or psychogenic cause. Opherk et al., “Ictal Heart Rate Differentiates Epileptic From Non-Epileptic Seizures” Neurology, 58(4):636-8 (2002).

Concurrent physiologic changes occurring with periodic leg movements during sleep (PLMS) are suspected to provide more sensitive indices of sleep fragmentation. Correlations of EEG, EKG and PLMS may be analyzed by visual scoring and spectral analysis. PLMS may result in a microarousal that is associated with an increase in EEG alpha activity. Conversely, PLMS that do not result in microarousal is associated with a significant increase in EEG delta and theta activity. PLMS, both with and without microarousal, induce a shortening of the EKG R-R interval (i.e., indicating tachycardia) but was more marked for leg movements associated with microarousal. Sforza et al., “EEG And Cardiac Activation During Periodic Leg Movements In Sleep: Support For A Hierarchy Of Arousal Responses” Neurology 52(4):786-91 (1999).

Carbamazepine efficacy following the administration of carbamazepine (400 mg) to relieve glossopharyngeal neuralgia, cardiac asystole and/or grand mal seizures is reflected in EEG-EKG recordings. Yang et.al., “Cardiac Syncope Secondary To Glossopharyngeal Neuralgia—Effectively Treated With Carbamazepine” J Clin Psychiatry, 39(10):776-8 (1978).

The usefulness of multimodal multitracer brain studies has been demonstrated by fusion and overlay of neuroimages with other types of scans. These analyses may be retrospective or concurrent, either automated or interactive, and may assist the diagnostic process in clinical situations. Pietrzyk et al., “Clinical Applications Of Registration And Fusion Of Multimodality Brain Images From PET, SPECT, CT, And MRI” Eur J Radiol, 21(3):174-82 (1996).

Benign diseases of the uterus can be evaluated by a combination of ultrasound, magnetic resonance imaging (MRI), hysterography, hysterosonography and hysteroscopy. Kinkel et al., “Value Of MR Imaging In The Diagnosis Of Benign Uterine Conditions” J Radiol, 81(7):773-9 (2000).

The integration of clinical, psychometric and electrophysiological evaluations in patients having Wilson's disease fail to show a correlation between psychometric and evoked potential abnormalities with a semiquantitative clinical score ranging from no (0) to severe (3) symptoms. The only significant correlation was found between the clinical total score and the time dependent psychometric tests. Thus, a high percentage of subclinical cerebral impairment detectable by acoustically evoked event related potentials do not correlate with the clinical status of the patients. Arendt et al., “The Diagnostic Value Of Multi-Modality Evoked Potentials In Wilson's Disease” Electromyogr Clin Neurophysiol, 34(3):137-48 (1994).

### Pharmaceutical Chemistry

The present invention contemplates pharmaceutical formulations including racemic or optically pure compounds that may be comprised in, but not limited to, powders, capsules, oral or intrapulmonary liquids, tablets, coated tablets, caplets, troches, dispersions, sustained release formulations suspensions, solution, patches and liquids. Young, U.S. Pat. No. 6,369,113 (hereby incorporated by reference). Alternatively, the formulations contemplated in the present invention may be administered intra-nasally; as for example, is known for optically pure (R)— or (S)-bupropion. Houdi et al., U.S. Pat. No. 6,150,420 (hereby incorporated by reference).

The above formulations may benefit from increasing the solubility of the drug during delivery to improve absorption. Hydrophilic drugs are usually easily soluble in the natural aqueous environment of a mammal. Hydrophobic drugs, however, are often difficult to dissolve in a manner that provides a steady and predictable delivery to the target organ. Common solubilizers for hydrophobic drugs include, but are not limited to, compounds that contain alcohols, glycols, or esters. Usually, the problem of solving the solubility of hydrophobic drugs involves mixtures containing triglyceride suspensions or colloids. These preparations are acceptable for topical administration but have obvious practical deficiencies when considering the oral or intrapulmonary or intravenous routes. In one embodiment, the present invention contemplates a formulation comprising hydrophobic and hydrophilic surfactants that coat a standard drug delivery device. In one non-limiting example, a bupropion formulation having the hydrophobic/hydrophilic coating is known to dissolve prior to the dispersal of the drug and provides an immediate environment that is highly favorable to solubilizing the drug to facilitate its absorption. Patel et al., U.S. Pat. No. 6,294,192 (hereby incorporated by reference).

The present invention contemplates embodiments having controlled delivery formulations. One example of a controlled delivery formulations is a semi-permeable homopolymer and copolymer film that is water-insoluble, yet water-permeable, and retains an active ingredient within an internal matrix. Preferably, the formulation contains a “water-permeability-modifying agent” within the polymers that changes the rate of osmosis through the polymer. This characteristic thereby controls the exit of the releasable active ingredient retained within the polymer film with the aid of an osmotic enhancing agent. Specifically, an osmotic enhancing agent is a water-soluble material having a high molar water solubility which is capable of achieving, in solution, an osmotic pressure greater than that of the surrounding aqueous environment. These films may be incorporated into standard pharmaceutical preparations such as, but not limited to, tablets, subdermal implants, suppositories, and capsules. An exemplary sustained release bupropion tablet is disclosed in Baker et al., U.S. Pat. No. RE33,994 (hereby incorporated by reference).

Bi/Tri-Layer Tablets

The present invention contemplates a multilayered tablet for the adminsitration of a pharmaceutical formulation as a compounded formulation. In one embodiment, the present invention contemplates a bilayer tablet having a first layer comprising an instant-release formulation of an anticonvulsant and a second layer comprising a sustained-release formulation of at least one neuroactive modulator. This type of bilayer tablet provides a fast and sustained therapeutic levels of any desired combination of pharmaceutical compounds. Blume et al., “Guaifensesin Sustained Release Formulation And Tablets” U.S. Pat. No. 6,372,252; and Richardson et al., “Dosage Forms For The Treatment Of The Chronic Glaucomas” U.S. Pat. No. 6,207,190 (both hereby incorporated by reference). In a non-limiting example, the present invention contemplates a bilayer tablet having the instant-release formulation comprising lithium carbonate and the sustained-release formulation comprising an anticonvulsant and a monoaminergic reuptake inhibitor. In a second non-limiting example, the present invention contemplates a bilayer tablet having the instant-release formulation and the sustained release formulation comprising an anticonvulsant and a monoaminergic reuptake inhibitor.

In one embodiment, the present invention contemplates a bilayer tablet having uniform release characteristics but containing two different active ingredients comprising the respective layers. For example, it is known that a bilayer tablet may consist of one layer of a non-steroidal anti-inflammatory agent while the second layer contains misoprostol. Woolfe et al., “Anti-Inflammatory Pharmaceutical Formulations” U.S. Pat. No. 6,319,519; and Ouali et al., “Stabilized Pharmaceutical Composition Of A Nonsteroidal Anti-Inflammatory Agent And A Prostaglandin” U.S. Pat. No. 6,287,600 (both hereby incorporated by reference). In a non-limiting example, the present invention contemplates a bilayer tablet wherein one layer comprises of an anticonvulsant and the second layer comprises of a monoaminergic reuptake inhibitor.

In a another embodiment, drug delivery from a bilayer tablet is enhanced wherein the active ingredients are present in the first layer and the second layer comprises of an osmotically active substance (i.e., for example, hydroxypropylmethylcellulose or a derivative thereof). The second layer expands in the presence of water and actively disburses the active ingredients comprising the first layer. Merrill et al., “Analgesic Tablet Composition” U.S. Pat. No. 6,284,274; and Singh et al., “Anti-Allergy Anti-Inflammatory Composition” U.S. Pat. No. 6,258,816 (both patents hereby incorporated by reference). In a non-limiting example, the present invention contemplates a bilayer tablet wherein the first layer comprises an anticonvulsant and a monoaminergic reuptake inhibitor and the second layer comprises hydroxypropylmethylcellulose.

A trilayer tablet is known that compounds two active ingredients, enalapril and losartan, such that enalapril is contained in the two outside layers to mask the bitter taste of the losartan in the middle layer. Chen et al., “Composition Of Enalapril And Losartan” U.S. Pat. No. 6,087,386 (hereby incorporated by reference). In one embodiment, the present invention contemplates a trilayer tablet wherein the first layer comprises an anticonvulsant; the second layer comprises a monoaminergic reuptake inhibitor; and the third layer comprises a drug.

Bi/Tri-Compartment Capsules

The present invention contemplates a multicompartment capsule for the adminsitration of a pharmaceutical formulation as a compounded formulation. In one embodiment, a bi-compartment capsule comprises a bilayer drug core that provides a more effective dispersal of the active ingredient. Preferably, the bi-compartment capsule contains a single active ingredient and a displacement layer (i.e., for example, sodium carboxymethylcellulose or a derivative thereof). Dong et al., “Progestin Tablet” U.S. Pat. No. 5,620,705 (hereby incorporated by reference). In a first non-limiting example, the present invention contemplates a bi-compartment capsule containing an anticonvulsant in a first compartment and a neuroactive modulator in a second compartment. In a second non-limiting example, the present invention contemplates a tri-compartment capsule containing an anticonvulsant in a first compartment, a monoaminergic reuptake inhibitor in a second compartment and a third drug in a third compartment.

Transdermal Patches

The present invention contemplates the transdermal delivery of pharmaceutical formulations provided by sustained and/or controlled release formulations. In one embodiment, the present invention contemplates the topical administration of pharmaceutical formulations to a patient's external epidermis. While it is not necessary to understand the mechanism(s) of the present invention, it is believed that transdermal delivery of pharmaceutical formulations will reduce the first pass metabolic hepatic effect on the production of metabolites. Although some pharmaceutical formulation metabolites are thought to have therapeutic effect, additional advantages of transdermal administration are expected to increase the bioavailability of the pharmaceutical formulation and improve therapeutic efficacy. Furthermore, it is believed that transdermal delivery will provide a continuous supply of any pharmaceutical formulation and maintain a stable, therapeutically effective level. Transdermal delivery of pharmaceutical formulations is considered more efficient than other modes of delivery (i.e., oral or intrapulmonary or intravenous) that are prone to provide a supratherapeutic concentration shortly after delivery that declines to a subtherapeutic concentration prior to the next dose.

Typically, any pharmaceutical formulation contained within a transdermal patch is incorporated onto a matrix or reservoir from which it is released onto the recipient's skin and ultimately passes into the patient's blood stream. The rate of release can be controlled by a membrane placed between the reservoir and the skin, by diffusion directly from the reservoir, or by the physical characteristics of the skin. In the simplest embodiment, the present invention contemplates that a suitable reservoir comprises, for example, a simple gauze pad impregnated with an active ingredient (i.e., for example, a formulation comprising an anticonvulsant and a neuroactive modulator) that is placed onto the skin in a secure manner. In one embodiment, the pharmaceutical formulation-containing reservoirs seal onto the skin of the patient. In this manner, the reservoir serves both as a repository for the active ingredient and as barrier to prevent loss or leakage of the substance away from the area of the skin to which the substance is to be delivered. In another embodiment, the transdermal patch further comprises a skin enhancer or penetration enhancer that facilitates the penetration of the pharmaceutical formulation through the external epidermal layers of the patient. Many penetration enhancers are known in the art, both water soluble and water insoluble. Audett et al., “Transdermal Delivery Of Basic Drugs Using Nonpolar Adhesive Systems And Acidic Solubilizing Agents” U.S. Pat. No. 5,879,701 (hereby incorporated by reference).

Monolithic transdermal patches may provide a stable delivery of therapeutic agents. For example, two basic systems rely on polyurethane acrylic copolymers as disclosed in To Szycher et al., “Drug Release System” U.S. Pat. No. 4,638,043; and Fischer et al., “Active Ingredient Patch” U.S. Pat. No. 5,830,505 (both of which are incorporated herein by reference). Another example of a transdermal patch employs an adhesive matrix of silicone and polyisobutylene either alone or in combination. Jona et al., “Transdermal Patch And Method For Administering 17-Deacetylnorgestimate Alone Or in Combination With An Estrogen” U.S. Pat. No. 5,876,746 (hereby incorporated by reference). A specific transdermal patch system intended for use on sensitive skin is disclosed in Gale et al., “Transdermal Drug Delivery Device Having Enhanced Adhesion” U.S. Pat. No. 5,840,327 (hereby incorporated by reference). In a first non-limiting example, the present invention contemplates a transdermal patch containing a daily divided dose of a formulation comprising an anticonvulsant and a neuroactive modulator. In a second non-limiting example, the present invention contemplates a transdermal patch containing a daily divided dose of a formulation comprising oxcarbazepine and bupropion. In a third non-limiting example, the present invention contemplates a transdermal patch containing a daily divided dose of a formulation comprising an anticonvulsant, a monoaminergic reuptake inhibitor, and a third drug, wherein the ratio of the doses may vary.

Transdermal patch therapy comprising bupropion is well known to alleviate withdrawal symptoms during the cessation of smoking cigarettes. This transdermal patch is constructed as an acrylic-based polymer pressure sensitive adhesive with a resinous cross-linking agent that is encased in a paper polyethylene-foil pouch. Cary, “Nicotine Addiction Treatment” U.S. Pat. No. 6,197,827 (hereby incorporated by reference). Other examples of bupropion-containing transdermal patches are disclosed in Midha et al., “Apparatus And Method For Transdermal Delivery Of Bupropion” U.S. Pat. No. 6,280,763, and Rose et al, “Method For Aiding In The Reduction Of Incidence Of Tobacco Smoking” U.S. Pat. No. 5,834,011 (both patents hereby incorporated by reference).

In another embodiment, the present invention contemplates long-term transdermal patch administration of a formulation comprising an anticonvulsant and a neuroactive modulator to the patient by exposing the patient's skin for an extended period of time; preferably from about 12 hours to 30 days, more preferably from about 24 hours to about 15 days, and most preferably from about 72 hours to about 7 days. Long-term transdermal delivery may also be more convenient than other modes of delivery and could increase patient compliance. Specifically, transdermal delivery may also be preferred because depressed patients may forget or avoid daily medication. Specifically, one embodiment of the present invention contemplates a transdermal delivery system that provides for a seven day administration period that coincides with weekly visits to a medical facility for a clinical evaluation with a simultaneous exchange of treatment patches.

Long-term transdermal administration of olanzapine, an antipsychotic, may be administered in combination with a skin enhancer (i.e., a C₂-C₆ alkanediol) for the treatment of psychosis, schizophrenia, mania or anxiety. This transdermal patch comprises primarily of a high capacity, polyurethane hydrogel reservoir comprised of a superabsorbent, crosslinked polymeric material capable of drug delivery for three to seven days. Jona et al., “Transdermal Administration Of Olanzapine” U.S. Pat. No. 5,891,461(hereby incorporated by reference). A weekly patch regimen (i.e., 140 hours) is also used for treatment of postmenopausal women using a trilayer patch for the simultaneous delivery of 17-β-estradiol and estrogen. Chien et al., “Transdermal Absorption Dosage Unit For Postmenopausal Syndrome Treatment And Process For Administration” U.S. Pat. No. 5,145,682 (hereby incorporated by reference). Multilayer patches are also disclosed for the transdermal administration of the S(+) enantiomer of desmethylselegiline for the treatment of depression and a variety of other disorders. DiSanto et al., “S(+) Desmethylselegiline And Its Use In Transdermal Delivery Compositions” U.S. Pat. No. 6,375,979 (hereby incorporated by reference).

Alternatively, transdermal delivery systems comprising reservoirs comprising ion exchange resins and amino acid polymers represent exemplary embodiments contemplated by the present invention. Bawa et al., “Sustained Release Formulation Containing An Ion-Exchange Resin” U.S. Pat. No. 4,931,279; and Bawa et al., “Sustained-Release Formulation Containing An Amino Acid Polymer” U.S. Pat. No. 4,668,506 (both patents hereby incorporated by reference). In a first non-limiting example, the present invention contemplates a transdermal patch containing a weekly dose of an anticonvulsant, a neuroactive modulator and a third drug, wherein the weekly dose may vary. In a second non-limiting example, the present invention contemplates a transdermal patch containing a weekly dose of a formulation comprising an anticonvulsant and a monoaminergic reuptake inhibitor.

Fast-Dissolve Formulations

The present invention contemplates treating a patient suffering from a nervous system disorder with a formulation comprising an anticonvulsant and a neuroactive modulator in a fast-dissolve, sublingual, formulation.

Although the present invention is not limited to any particular mechanism, it is believed that the adjustment of the pH of the environment of the sublingual area may improve the absorption of the therapeutic formulation. It is contemplated that the fast dissolve formulation comprise at least one component the will adjust the pH of the local environment of the sublingual area.

Sublingual administration of a fast dissolve formulation may take many forms. In one embodiment, the formulation is a tablet or packed powder. In another embodiment, the fast dissolve formulation may comprise a medical device such as a patch. The patch may be placed under the tongue. The patch may have adhesive qualities to prevent the movement, loss or swallowing of the patch. The patch may be ingestible in case of accidental swallowing or to allow for easy disposal of the patch. In another embodiment, the patch may be removed from under the tongue after the prescribed time. In yet another embodiment, the fast dissolve formulation may take the form of a paste or gel, wherein the paste or gel would be applied under the tongue. The viscosity of the paste or gel can be adjusted to allow for the retention under the tongue. In another embodiment, it is contemplated that the present invention is a liquid. It is further contemplated that the liquid is in the form of a spray or drops.

Another fast dissolve formulation contemplated by the present invention comprises a hard, compressed, rapidly dissolving tablet adapted for direct sublingual dosing. The tablet comprises particles made of an active ingredient and a protective material. These particles are provided in an amount of between about 0.01 and about 75% by weight based on the weight of the tablet. The tablet may also include a matrix made from a nondirect compression filler, a wicking agent, and a hydrophobic lubricant. The preferred tablet matrix comprises at least about 60% rapidly water-soluble ingredients based on the total weight of the matrix material. The preferred tablet has a hardness of between about 15 and about 50 Newtons, a friability of less than 2% when measured by U.S.P. and is adapted to dissolve spontaneously in the mouth of a patient in less than about 60 seconds (and, more preferably, less than about 30 seconds) and thereby liberate the particles and be capable of being stored in bulk.

In yet another embodiment, the compressed rapidly dissolving tablet comprises effervescent agents. These effervescent agents allow enhanced adsorption of the pharmaceutical formulation across the mucosal membranes in the sublingual cavity. An example of effervescent pharmaceutical formulations suitable for use in conjunction with the present invention are the compositions described in Pather, U.S. Pat. No. 6,200,604 (hereby incorporated by reference). Other pharmaceutical formulations suitable for use in conjunction with the present invention are the compositions described in Wehling, et al., U.S. Pat. No. 5,178,878 & U.S. Pat. No. 5,223,264; and to Khankari et al. U.S. Pat. No. 6,024,981 (all three patents are hereby incorporated by reference).

Microparticles

One aspect of the present invention contemplates a microparticle comprising a pharmaceutical formulation. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Microspheres and microcapsules are useful due to their ability to maintain a generally uniform distribution, provide stable controlled compound release and are economical to produce and dispense. One skilled in the art should recognize that the terms “microspheres, microcapsules and microparticles” (i.e., measured in terms of micrometers) are synonymous with their respective counterparts “nanospheres, nanocapsules and nanoparticles” (i.e., measured in terms of nanometers). It is also clear that the art uses the terms “micro/nanosphere, micro/nanocapsule and micro/nanoparticle” interchangeably, as will the discussion herein.

Microspheres

In one embodiment, the present invention contemplates a pharmaceutical formulation comprising microspheres. Preferably, polysaccharide microspheres may be used including those which carry suitable anionic groups such as carboxylic acid residues, carboxymethyl groups, sulphopropyl groups and methylsulphonate groups or cationic groups such as amino groups. For example, carboxylated starch microspheres are available from Perstorp (Sweden). Other suitable materials for the microspheres include hyaluronic acid, chondroitin sulphate, alginate, heparin and heparin-albumin conjugates. Kwon et al., Int. J Pharm. 79:191 (199)

In other embodiments, microspheres may comprise materials including, but not limited to, carboxymethyl dextran, sulphopropyl dextran, carboxymethyl agarose, carboxymethyl cellulose, cellulose phosphate, sulphoxyethyl cellulose, agarose, cellulose beads or dextran beads. (all of which are commercially available).

The present invention contemplates methods of making microspheres comprising spray drying, coacervation and emulsification. Davis et al. “Microsphere and Drug Therapy” Elsevier, 1984; Benoit et al. “Biodegradable Microspheres: Advances in Production Technologies” Chapter 3, Ed. Benita, S, Dekker, New York (1996); In: Microencapsulation and related Drug Processes, pp 82, 181 and 225, Ed. Deasy, Dekker, New York (1984); Green et al., U.S. Pat. No. 2, 730,457; and Evans et al., U.S. Pat. No. 3,663,687 (both patents hereby incorporated by reference).

In the spray drying process, the material used to form the body of the microsphere is dissolved in a suitable solvent (usually water) and the solution spray dried by passing it through an atomization nozzle into a heated chamber. The solvent evaporates to leave solid particles in the form of microspheres.

In the process of coacervation, microspheres can be produced by interacting a solution of a polysaccharide carrying a positive charge with a solution of a polysaccharide carrying a negative charge. The polysaccharides interact to form an insoluble coupling that can be recovered as microspheres.

In the emulsification process, an aqueous solution of the polysaccharide is dispersed in an oil phase to produce a water in oil emulsion in which the polysaccharide solution is in the form of discrete droplets dispersed in oil. The microspheres can be formed by heating, chilling or cross-linking the polysaccharide and recovered by dissolving the oil in a suitable solvent.

The microspheres can be hardened before incorporating a pharmaceutical formulation by cross-linking procedures such as heat treatment or by using chemical cross-linking agents. Suitable crosslinking agents include, but are not limited to, dialdehydes, including glyoxal, malondialdehyde, succinicaldehyde, adipaldehyde, glutaraldehyde and phthalaldehyde, diketones such as butadione, epichlorohydrin, polyphosphate or borate. In one embodiment, a dialdehydes cross-links protein amino groups and diketones to form Schiff bases. In another embodiment, epichlorohydrin converts compounds with nucleophilic centers such as amino or hydroxyl to epoxide derivatives.

In one embodiment, a pharmaceutical formulation may be incorporated into a microsphere at different ratios. In one example, the ratio (weight-to-weight) of microsphere material to pharmaceutical formulation is greater than one. It should be understood by those skilled in the art that the proper microsphere ratio may be dictated by the required drug dosage the complexation properties of the microsphere material.

In one embodiment, a microparticle contemplated by this invention comprises a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

In another embodiment, a microparticle further comprises a pharmaceutical formulation comprising an anticonvulsant and a neuroactive modulator directly bound to the surface of the microparticle or is indirectly attached using a “bridge” or “spacer”. The amino groups of the gelatin lysine groups are easily derivatized to provide sites for direct coupling of the formulation. Alternatively, spacers (i.e., linking molecules and derivatizing moieties on targeting ligands) such as avidin-biotin are also useful to indirectly couple targeting formulations to the microparticles. Stability of the microparticle may be controlled by the amount of glutaraldehyde-spacer crosslinking. A controlled release microparticle may be empirically determined by the final density of glutaraldehyde-spacer crosslinks.

Liposomes

In one embodiment, the present invention contemplates a pharmaceutical formulation contained with liposomes. Liposomes are spherical, self-closed structures composed of lipid bilayers which entrap in their interior compounds, such as, but not limited to, pharmaceutical formulations. In one embodiment, a liposome may consist of one or more concentric membranes. In another embodiment, a liposome may range in size from several nanometers to several dozens of micrometers.

Liposomes are mostly made from amphiphilic molecules which can be characterized by having a hydrophilic (often named the polar head) and a hydrophobic group (nonpolar tail) on the same molecule. In most cases, liposome-forming molecules are not soluble in water. However, under certain circumstances, they form colloidal dispersions.

Liposomes can be large or small and may be composed from one to several hundred of concentric bilayers. With respect to the size and the nature of the layer (lamellae), liposomes are classified as multi-lamellar vesicles (MLVs), small uni-lamellar vesicles (SUVs) and large uni-lamellar vesicles (LUVs). Specifically, SUVs have a diameter from approximately 20 nm to 600 nm and consist of a single lipid bilayer which surrounds the interior aqueous compartment. On the other hand, LUVs have a diameter from approximately 600 nm to 3000 nm. Finally, MLVs vary greatly in size from approximately 3000 nm to 10,000 nm and comprise at least two lipid bilayers.

The present invention contemplates various embodiments regarding methods of making liposomes. In one embodiment (the “thin-film hydration” method) comprises heterogeneous dispersions of predominantly MLVs. In another embodiment, charged lipid compositions result in predominantly LUVs. In another embodiment, SUVs are produced by treating lipid dispersions by methods known in the art including mechanical agitation, electrostatic exposure or chemical treatments. Preferably, these methods further comprise extrusion through filters with pores of different diameter, or sonication.

Another embodiment contemplates the production of liposomes comprising lyophilization. In one embodiment, a lipid-film is dissolved in a volatile solvent (i.e., for example, tert-butyl alcohol), frozen and lyophilized. Szoka et al., In: Ann. Rev. Biophys. Bioeng. 9, 467-508 (1980); Schneider, et al. U.S. Pat. No. 4,229,360; Papahadjopoulos, et al., U.S. Pat. No. 4,241,046; and Papahadjopoulos, et al., U.S. Pat. No. 4,235,871 (all three patents hereby incorporated by reference).

Injectables

The present invention contemplates the administration of drugs by a method comprising injection (i.e., for example, with a single syringe or intravenous catheter). In one embodiment, injection of a pharmaceutical formulation comprising an anticonvulsant and a neuroactive modulator includes, but is not limited to, intravenous, subcutaneous, intradermal or intraperitoneal. The dose ranges of each type of injection varies with the specific formulation involved that are well known to those skilled in the art. In particular, the injectable solutions are sterile and comprise buffers, salts and other compounds to reduce irritation or side effects.

Intra-Nasal Administration

Pharmaceutical formulations contemplated by the present invention are contemplated for administration from a nasal spray comprising a solution. In one embodiment, the solution is hydrophilic. In another embodiment, the solution is hydrophobic. Systems for dispensing liquids as a spray are known in the art. Kalat, E. F., U.S. Pat. No. 4,511,069 (hereby incorporated by reference). In one embodiment, a nasal spray comprises a pharmaceutical formulation, a non-ionic surfactant, polysorbate-80, and one or more buffers. In another embodiment, the nasal spray further comprises a second non-ionic surfactant including, but not limited to, nonoxynol-9, laureth-9, poloxamer-124, octoxynol-9 or lauramide DEA. In some embodiments of the present invention, the nasal spray solution further comprises a propellant. Preferably, the pH of the nasal spray solution is between approximately pH 6.0-8.0, more preferably between pH 6.5-7.5, but more preferably between pH 6.8 and 7.2.

The desired concentration of the drug or drugs in compositions according to the present invention, can be readily determined by those skilled in the art of pharmacology.

Intra-Pulmonary Administration

In one embodiment, a pharmaceutical formulation comprising an anticonvulsant and a neuroactive modulator is administered by a method comprising pulmonary administration. In one embodiment, the pulmonary administration is by aerosolization. Preferably, a pharmaceutical formulation for aerosolized pulmonary administration is comprised such that the formulation is pharmacologically active following delivery to the lungs. In one embodiment, the formulation contains diluents, adjuvants or excipients, among other things. In one, a formulation comprising an anticonvulsant and a neuroactive modulator is dissolved in a sterile liquid vehicle. The term “sterile liquid vehicle” refers to those liquids that are suitable for administration to a patient (e.g., pulmonary or parenteral administration) and allow dissolution of the formulation. Examples of sterile liquid vehicles include, but are not limited to, sterile normal saline and dilute concentrations of ethanol.

In one embodiment, the administration comprises administration to the lung. Patients having nervous system disorders who require mechanical ventilation may continue to receive treatment with pharmaceutical formulations administered via the endotracheal tube which is connected to the ventilator. Alternatively, the formulation may be administered to the lung through a bronchoscope.

### Pharmaceutical Formulation Dispensation Devices

The present invention contemplates a device having the ability to dispense solid dosage pharmaceutical formulations. In one embodiment, the dispensing device is marked to allow the patient, or medical personnel, to determine which dosage requires taking at any particular time and, further, determining if any past dosages were not taken. In another embodiment, the present invention contemplates a dispensing device capable of dispensing a plurality of different formulations simultaneously.

In one embodiment, the present invention contemplates a restricted access device capable of a single dispensation of a present dosage formulations while preventing access to future dosage formulations. For example, in one embodiment, a restricted access device comprises a tray capable of dispensing a single tablet. Upon depressing and pushing forward a locking member, the tray slides out from the tablet container to allow access to, and administration of, the present dosage formulation. The tray is then slid back into the tablet container and the tray is automatically refilled with the next future dosage formulation. Kozlowski et al., “Child-Proof Tablet Dispenser” U.S. Pat. No. 5,174,471 (hereby incorporated by reference).

In one embodiment, a restricted access device may lack a locking mechanism. For example, in one embodiment, a restricted access device comprises a tablet container capable of individually dispensing single tablets simply by activating an opening device. In one embodiment, releasing the opening device closes the container and simultaneously positions a future dosage formulation in a dispensable position. Bar-Yona et al., “Tablet Dispenser” U.S. Pat. No. 5,351,858 (hereby incorporated by reference).

In another embodiment, a restricted access device comprises a blister package containing a plurality of pharmaceutical formulations. In one embodiment, the blister package comprises a plastic dome structure that retains a pharmaceutical formulation on the surface of a backing material. One advantage of this device is that patient non-compliance is easily determined as the unadministered pharmaceutical formulation is visible within the blister package following the indicated administration day. In one embodiment, a blister package comprises a single formulation or a plurality of formulations capable of identifying administration on a daily basis. Leonard et al., “Calendar-Oriented Pill Dispenser” U.S. Pat. No. 4,736,849 (hereby incorporated by reference). In one embodiment, blister packages organize identical tablets by rows. In another embodiment, the row organization of identical tablets are marked on the backing comprising a coding system that results in the specific identification of each formulation present on the blister package. In one embodiment, the blister package comprises a coding system that references days, months, and years.

The present invention contemplates a controlled access device comprising a plurality of pharmaceutical formulations. In one embodiment, the device comprises a circular tray having concentric ring arrangements of tablet compartments. In another embodiment, the tray comprises an annual, monthly or weekly arrangement of multiple dosage forms. In one embodiment, the diameter of the inner concentric ring compartments are smaller than the diameter of the outer concentric ring compartments such that pharmaceutical formulations of both the inner and outer concentric ring compartments intended for administration on the same day are adjacent. In one embodiment, the controlled access device comprising an inner and outer concentric ring compartments is capable of dispensing two tablets for twenty-eight days. Pierantozzi et al, “Pharmaceutical Tablet Dispenser” U.S. Design Pat. No. 335,081 (hereby incorporated by reference). In another embodiment, a controlled access device comprises a dual shelf dispenser capable of dispensing two tablets for twenty-five days. Walchek et al. U.S. Design Pat. No. 358,762 (hereby incorporated by reference).

In one embodiment, a controlled access device comprises sealed packets enclosing a plurality of pharmaceutical formulations. In one embodiment, rotation of a compartment to align with an outer concentric ring aperture breaks the sealed packet thus releasing the plurality of pharmaceutical formulations such that the formulations exit the device. Studer, “Tablet Dispenser” U.S. Pat. No. 4,165,709; and Lambelet et al., “Variable Day Start Tablet Dispenser” U.S. Pat. No. 6,138,866 (both patents hereby incorporated by reference).

In one embodiment, a controlled access device comprises a circular tray having adjustable pre-set indicators for day-of-week administration starting on any specific day of the week. In one embodiment, the tray is rotated until the desired start day appears in a window. In another embodiment, the start-day alignment automatically arranges the sealed compartment dosage formulations to line-up with the proper week-day of their administration. Richardson et al., “Tablet Dispenser” U.S. Pat. No. 3,651,927 (hereby incorporated by reference).

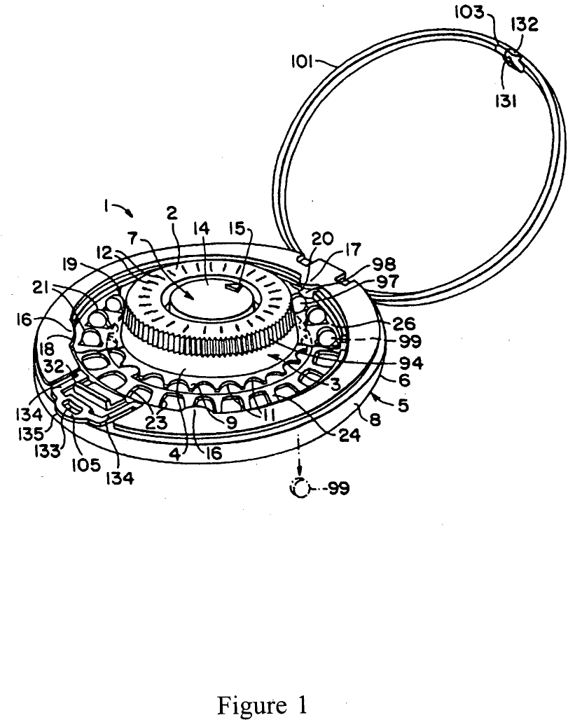

FIG. 1 illustrates one exemplary design of a tablet dispensing device contemplated by this invention as a perspective view of a tablet dispenser 1. The tablet dispenser 1 comprises as a first component, a substantially circular unidirectional rotatable knob 3 which is encircled with a notched skirt 9 comprising a plurality of notches 11 spaced substantially equally apart. The rotatable knob 3 comprises a flat surface 2 and a cylindrical wall 4. A portion of the cylindrical wall 4 may be provided with ridges 94 in a knurling pattern for enhancing hand gripping of the rotatable knob 3. The rotatable knob 3 is mounted onto a second component, which is base 5 comprising a substantially flat support 6, having a single tablet dispensing aperture 13, and a rising wall 8 extending from the periphery of the flat support 6.

The rotatable knob 3 is attached to the flat support by engagement means around a third component which is a fixed center axis means 7 about which the rotatable knob 3 may be rotated in a circular fashion. The fixed center axis means 7 has a flat top 14 and includes an optimal pointer shaped indicator 15 which aligns with an angular ledge 17, a current or initial tablet position 97 and a corresponding day of administration 12 imprinted on the flat surface 2 of the rotatable knob 3.

The tablet dispenser shown in FIG. 1 comprises a fourth component which is a separate and removable tablet package 19 which is adapted to fit over the rotatable knob 3 with means to positively engage the notched skirt 9 thereof such that the two components rotate in unison. The separate and removable tablet package 19 comprises a rigid platform 24 and an essentially flexible blister ring By 26 upon which tablets 99 are provided in collapsible tablet pockets 21. The tablet package 19 comprises a plurality of collapsible tablet pockets 21 each containing a tablet 99 arranged substantially circularly about the package whereby the spacing of the tablet pockets 21 correspond to each stop of the ratchet means, whereby a new tablet 99 is placed over the tablet dispenser aperture 13 upon the positive engagement of each stop on the ratcheted rotatable knob 3. The tablet pockets 21 are lidded with a frangible membrane 22 which is sealed to the blister ring 26 and interposed between the tablets 99 in the tablet pockets 21 and a single tablet dispensing aperture 13. A substantially rigid or stiff platform 24 comprises a plurality of tablet apertures 23 which are substantially aligned with each tablet pocket 21. A tablet 99 is dispensed from the tablet dispenser 1 by collapsing the tablet pocket 21 which is in registry with the single tablet dispensing aperture 13 thereby forcing the tablet to fracture a frangible membrane 22 and pass through the apertures 23 and 13. The rigid platform 24 and the flexible blister ring 26 are held together by bonding means (e.g. glue, ultrasonic welding or staking).

The base 5 has a rising wall 8 extending from the flat support 6 to form a cup like interior space in which the rotatable knob 3 and tablet package 19 are housed. The base 5 comprises at least two inwardly extending ledges 16 protruding from the rising wall portion 8 toward the center axis means 7. The shape and the orientation of the ledges 16 correspond to at least two complementary recesses 18 on the tablet package 19 permitting reception of the tablet package 19 onto the flat support 6, whereby a designated first tablet 97 is positioned above the tablet dispensing aperture 13 at the initial or current tablet position 98 which is indicated by an angular ledge 17. The angular ledge 17 may be cooperative with ledges 16 by corresponding to complementary recesses 20 and 18 of the tablet package 19 to provide reception of the tablet package 19 onto the flat support 6. The tablet package 19 is interlocked onto the base 5 upon a single advance of the calendared rotatable knob 3 whereby a portion of the rigid platform 24 underlaps the inwardly extending ledges 16 and 17. The tablet package is not disengageable or removable until a significant rotation of the knob 3 returns the tablet package 19 to the initial tablet position 98. A finger lever 32 is provided, diametrically opposite the angular ledge 17.

The tablet package further comprises a cover 101 which together with the base 5 protects the dispenser contents from impact damage and light degradation particularly where the base and cover material is of such density and opacity as to filter out degradative wavelengths of light and to protect the dispenser's contents from physical damage attendant to normal use. A latch strut 103 extends toward the base 5 from the cover 101. The latch strut 103 comprises an inward hook 131 and an outward lever 132. When the cover 101 is closed onto the base 5, the latch strut 103 passes through a latch seat aperture 133 into a cavity beneath latch seat 105 thereby snapping the inward hook 131 beneath the bottom surface of the latch seat 105 and abutting the outward lever 132 to the top surface of the latch seat. The latch seat 105 is connected to the base 5 by torsion arms 134 such that latch lever 135 overhangs the base. To open the dispenser, the latch lever 135 is urged upward thereby lifting the outward lever 132 while rotating the seat aperture 133 into disengagement from the inward hook 131 resulting in the cover springing ajar.

In one embodiment, a controlled access device comprises vertical chambers that rotate along an axial plane. In one embodiment, the device organizes the pharmaceutical formulations according to particular days of the week. In one embodiment, the vertical chamber device comprises a seven-sided housing containing seven chambers (color coded for each day of the week) capable of vertically storing a plurality of pharmaceutical formulations. In another embodiment, the vertical chamber device is capable of storing four weeks of tablets that are capable of individual dispensation by rotating the housing to the proper day setting and sliding the week-indicator to the proper level. Rappaport et al., “Pill Dispenser Providing Sequential Dispensing Means And Automatic Incremental Dispensing Control” U.S. Pat. No. 4,807,757 (hereby incorporated by reference).

An alternative design for a controlled access device comprises a bottle containing a pre-determined order of tablets that is placed onto a rotatable cap. In one embodiment, the cap is rotated wherein a single tablet is dispensed. In one embodiment, the cap rotation further comprises advancing an indicator to the next pharmaceutical formulation. Robbins, “Dispensing And Recording Container” U.S. Pat. No. 3,678,884 (hereby incorporated by reference).

The present invention contemplates electronic reminder and tracking systems to properly administer a plurality of pharmaceutical formulations. In one embodiment, a housings comprises rows and columns of pillboxes wherein an electronic indicator grid identifies the proper pillbox, time, and day. Blum, “Pill Dispenser” U.S. Pat. No. 4,640,560; and Newland, “Medication Storage And Reminder Device” U.S. Pat. No. 6,169, 707 (both patents hereby incorporated by reference).

One advantage of the present invention contemplates a device for a predetermined dispensation of separate formulations of an anticonvulsant and a neuroactive modulator during a one month time interval. In one embodiment, the predetermined dispensation comprises oxcarbazepine formulations of gradually increasing daily doses and bupropion formulations of gradually decreasing daily doses during a one month time interval, wherein oxcarbazepine and bupropion are separate formulations. In another embodiment, the predetermined dispensation comprises a bilayer formulation comprising a first layer having gradually increasing daily dose of oxcarbazepine and a second layer having gradually decreasing daily dose of bupropion during a one month time interval. In one specific embodiment, the predetermined dispensation comprises a daily divided dose between oxcarbazepine and bupropion, wherein the daily divided dose includes, but is not limited to, 4000/25, 3700/75, 3400/125, 3100/175, 2800/325, 2500/375, 2200/425, 1900/475, 1600/525, 1300/575, 1000/625, 700/675, 400/725 or 150/750 milligrams.

Another advantage of the present invention contemplates a device for the predetermined dispensation of pharmaceutical formulations of a compounded anticonvulsant/neuroactive modulator and a selective serotonin reuptake inhibitor (SSRI) during a one month period. In one embodiment, the formulation comprises a gradual increase in the daily dose of a compounded oxcarbazepine/bupropion and a gradual decrease in the daily dose of an SSRI formulation during a one month period. In one embodiment, the compounded anticonvulsant/neuroactive modulator formulation is evenly mixed (i.e., uniform), wherein the formulation is selected from the group comprising a tablet or a capsule. In another embodiment, the compounded anticonvulsant/neuroactive modulator formulation is not evenly mixed (i.e., non-uniform), wherein the formulation is selected from the group comprising a multilayer tablet or a multi-compartmental capsule. In one embodiment, a daily divided dose ratio of a compounded oxcarbazepinelbupropion formulation includes, but is not limited to, 4000/25, 3700/75, 3400/125, 3100/175, 2800/325, 2500/375, 2200/425, 1900/475, 1600/525, 1300/575, 1000/625, 700/675, 400/725 or 150/750 milligrams. In one embodiment, a daily divided dose of the selective serotonin inhibitor ranges between approximately 5-450 milligrams.

Another advantage of the present invention contemplates a device for the predetermined dispensation of a pharmaceutical formulation comprising a selective serotonin reuptake inhibitor (SSRI), an anticonvulsant and a neuroactive modulator during a one month period. In one embodiment, the formulation comprises a gradual decrease in the daily dose of an SSRI, a gradual increase in the daily dose of oxcarbazepine, and a gradual increase in the dose of bupropion during a one month period. In one embodiment, the formulation is evenly mixed (i.e., uniform), wherein the formulation is selected from the group comprising a tablet or a capsule. In another embodiment, the formulation is not evenly mixed (i.e., non-uniform), wherein the formulation is selected from the group comprising a multilayer tablet or a multi-compartmental capsule. In one embodiment, a daily divided dose of the SSRI is with a range of approximately 5-450 milligrams. In one embodiment, a daily divided dose of the oxcarbazepine is within a range of approximately 4000-150 milligrams. In another embodiment, a daily divided dose of the bupropion is within a range of approximately 25-750 milligrams.

Experimental

The following examples serve to illustrate certain preferred embodiments and advantages of the present invention and are not to be construed as limiting the scope thereof.

### EXAMPLE 1

### Treatment of a Nervous System Disorder Using a Bupropion/Oxcarbazepine Formulation

This example provides an illustration of the expected effectiveness of the bupropion/oxcarbazepine formulation in alleviating at least one symptom of a nervous system disorder.

The design of this study is a randomized double-blind protocol in which a first set of clinicians diagnosed a group of naive (i.e., previously untreated) patients presenting at least one symptom of a nervous system disorder. The first set of clinicians will then randomly assign the patients to one of three treatment groups:
Group I: placebo;
Group II: selective serotonin reuptake inhibitor;
Group III: bupropion;
Group IV: oxcarbazepine; and
Group V: bupropion/oxcarbazepine.

A second set of clinicians will monitor the compliance of each patient and assess the presence or absence of at least one symptom of a nervous system disorder on a weekly basis throughout the treatment period. At the termination of the study a third set of clinicians will evaluate the data and document the results.

As predicted in Table I, Group V will demonstrate a greater reduction in at least one nervous system disorder symptom versus Group II, III or IV. Relative to Group I, all treatment groups are expected to reduce at least one symptom of a nervous system disorder except Group IV.

TABLE I
Percent Reduction In Nervous System Disorder Symptoms
In Affected Patients

| Symptom | Group I | Group II | Group III | Group IV | Group V |
|---|---|---|---|---|---|
| ONE | 0 | 50 ± 5 | 25 ± 2.5 | 10 ± 1 | 75 ± 7.5 |
| TWO | 0 | 50 ± 5 | 25 ± 2.5 | 10 ± 1 | 75 ± 7.5 |
| THREE | 0 | 50 ± 5 | 25 ± 2.5 | 10 ± 1 | 75 ± 7.5 |
| FOUR | 0 | 50 ± 5 | 25 ± 2.5 | 10 ± 1 | 75 ± 7.5 |
| FIVE | 0 | 50 ± 5 | 25 ± 2.5 | 10 ± 1 | 75 ± 7.5 |
| SIX | 0 | 50 ± 5 | 25 ± 2.5 | 10 ± 1 | 75 ± 7.5 |
| SEVEN | 0 | 50 ± 5 | 25 ± 2.5 | 10 ± 1 | 75 ± 7.5 |
| EIGHT | 0 | 50 ± 5 | 25 ± 2.5 | 10 ± 1 | 75 ± 7.5 |
| * - Greater | | * Group I | * Group I | | *Group I |
| Symptom | | *Group III | *Group IV | | *Group II |
| Reduction | | *Group IV | | | *Group III |
| | | | | | *Group IV |

This data will show that the formulation of oxcarbazepine and bupropion is most effective in reducing at least one symptom of a nervous system disorder.

### EXAMPLE 2

### Treatment of Non-Remissive Nervous System Disorders Using a Bupropion/Oxcarbazepine Formulation

This example will provide an illustration of the effectiveness of the bupropion/oxcarbazepine formulation in alleviating at least one symptom of a nervous system disorder that is non-remissive to a third drug protocol.

The design of this study is a randomized double-blind protocol in which a first set of clinicians identifies a group of non-remissive patients being administered a selective serotonin reuptake inhibitor and presenting at least one symptom of a nervous system disorder. Optionally, neurophysiological data will be collected including, but not limited to, EEG data compatible with QEEG analysis software. It is expected that this QEEG analysis will be useful as a biomarker for the administered formulation. The first set of clinicians will then randomly assign the patients to one of three treatment groups:
Group I: placebo;
Group II: selective serotonin reuptake inhibitor;
Group III: bupropion;
Group IV: oxcarbazepine; and
Group V: bupropion/oxcarbazepine.

A second set of clinicians will then monitor compliance of each patient and assess the continued presence of at least one symptom of a nervous system disorder on a weekly basis throughout the treatment period. At the termination of the study a third set of clinicians will evaluate the data and document the results.

As illustrated in Table II, Group V will demonstrate a greater reduction in at least one symptom of a nervous system disorder versus Group II, III or IV. Relative to Group I, Group III and Group V also are expected to reduce at least one symptom of a nervous system disorder.

TABLE II
Percent Reduction In Nervous System Disorder Symptoms
In SSRI-Refractory Patients

| Symptom | Group I | Group II | Group III | Group IV | Group V |
|---|---|---|---|---|---|
| ONE | 0 | 10 ± 1 | 25 ± 2.5 | 10 ± 1 | 75 ± 7.5 |
| TWO | 0 | 10 ± 1 | 25 ± 2.5 | 10 ± 1 | 75 ± 7.5 |
| THREE | 0 | 10 ± 1 | 25 ± 2.5 | 10 ± 1 | 75 ± 7.5 |
| FOUR | 0 | 10 ± 1 | 25 ± 2.5 | 10 ± 1 | 75 ± 7.5 |
| FIVE | 0 | 10 ± 1 | 25 ± 2.5 | 10 ± 1 | 75 ± 7.5 |
| SIX | 0 | 10 ± 1 | 25 ± 2.5 | 10 ± 1 | 75 ± 7.5 |
| SEVEN | 0 | 10 ± 1 | 25 ± 2.5 | 10 ± 1 | 75 ± 7.5 |
| EIGHT | 0 | 10 ± 1 | 25 ± 2.5 | 10 ± 1 | 75 ± 7.5 |
| * - Greater | | | * Group I | | *Group I |
| Symptom | | | *Group II | | *Group II |
| Reduction | | | *Group IV | | *Group III |
| | | | | | *Group IV |

This data will show that a pharmaceutical formulation comprising oxcarbazepine and bupropion, is most effective in reducing the symptoms of a nervous system disorder.

### EXAMPLE 3

### Type One QEEG Analysis

An EEG is administered to a patient using a commercially available EEG instrument (Cadwell Laboratories, Bio-Logic Systems, Inc., Nicolet Biomedical or Oxford Instruments). Electrodes are placed on the patient's scalp using the International 10/20 System convention for determining the appropriate location of the electrodes. The raw EEG information is then stored in a digital format for subsequent FFT processing.

The following patient criteria are operative for Type One Analysis. The patient must be between the ages of 6 and 90 years. In addition, for Type One Analysis the patient must not be undergoing drug therapy. This is because all pharmacological agents (i.e., for example, drugs) may influence EEG information and give rise to false data. “Drugs” include those obtained by prescription or “on-the-street”, over-the-counter sleeping pills, pain medications, nutriceuticals and vitamins. It the patient is undergoing drug therapy, the therapy must be discontinued or avoided for seven half lives prior to the EEG test. However, the patient may be undergoing hormone replacement therapy for insulin, thyroid, progesterone and estrogen, as well as for other hormonal deficiencies.

A variety of patients are not suitable for Type One Analysis. These include individuals who have undergone intramuscular depo-neuroleptic therapy within the preceding twelve months. Individuals who have a history of craniotomy with or without metal prosthesis or have current unstable seizure disorder, dementia, and mental retardation are also not candidates for Type One Analysis. Individuals who are currently using marijuana, cocaine, hallucinogens, or other illicit psychotropic compounds are not candidates for Type One Analysis. Individuals with a significant metabolic abnormality (e.g., CBC, chemistry or thyroid difficulties) are not candidates for Type One Analysis until these systemic processes have been normalized.

The EEG information collected from the individuals is then digitized, subjected to FFT processing and analyzed. The first stage of analysis involves extracting a standard set of quantitative univariate measures from the FFT processed EEG information. These quantitative measures include, but are not limited to, absolute power and relative power. Absolute power is believed to be the square of the signal amplitude, measured in microvolts squared (i.e., V²). Relative power is believed to be the proportion of power in a given frequency band detected at a given electrode compared to the total band power detected at that electrode. There are at least four EEG frequency bands useful in QEEG analysis: delta (0.5-3.5 Hz); theta (3.5-7.5 Hz); alpha (7.5-12.5 Hz); and beta (12.5-35 Hz). The total EEG spectrum therefore runs from 0.5-35 Hz. The method of the current invention is not limited to these frequency bands and can be applied to any frequency banding.

One other useful univariate data parameter extracted during the first stage of QEEG analysis is coherence. It is believed that coherence measures the similarity for two scalp electrodes for all interhemispheric and intra-hemispheric electrode pairs, for each of the defined frequency bands. Peak frequency measures are also computed within each frequency band. Finally, the combination of power and coherence measures may be computed for defined sets of scalp electrodes.

### EXAMPLE 4

### Classification of EEG/QEEG Drug Response

A database of drug-free patients containing EEG/QEEG univariate data parameters and subsequent pharmacological treatment efficacies were compiled over a nine year period. A rule-based classifier using the current individual patient's neurophysiologic information profile and the database from the patient population was used to review pretreatment EEG/QEEG information from each study patient. An EEG/QEEG specific drug response prediction was reported to the patient control officer. This information was distributed only to the treating physician of the individual patient. Drug therapy response predictions for all other patients were sealed until the end of the study.

An antidepressant responsive spectrum identified in previous studies was incorporated in the rule-based classifier used to predict anti-depressant responsivity. The average relative power spectrum (i.e., containing QEEG multivariable composite Z-scores) of sixty responsive patients with affective and attentional disorders was analyzed. The spectrum demonstrated a global delta frequency deficit from −2.5 to −1.8 mean-units extending posteriorly, a diffuse theta deficit trend of −0.8-1.0 mean-units sparing the temporal or intrapulmonary regions, a +2.3 mean-units alpha maximum in the frontal polar region and a second alpha maximum of +2.1 mean-units in the posterior frontal region. These maxima are accompanied by a relative alpha minimum of +1.2 mean-units in the temporal or intrapulmonary region and sustained posterior alpha excess.

A stimulant responsive spectrum identified in previous studies was incorporated in the rule-based classifier used to predict stimulant responsivity for all study patients. The average relative power spectrum (i.e., containing QEEG multivariable composite Z-scores) of twenty-one responsive patients with affective and attentional disorders was analyzed. This spectrum exhibited a frontal polar delta frequency deficit from −2.0 to −2.3 mean-units. There were two frontal maxima in the theta band at +2.6 and +2.5 mean-units. The theta frequency showed +1.7 mean-units excess in the temporal or intrapulmonary region, gradually diminishing posteriorly toward +0.9 mean-units. The alpha and beta bands of this spectrum were distributed about a mean-score of zero.

An anticonvulsant/lithium response spectrum (data not shown) was incorporated in the rule-based classifier used to predict combination anticonvulsant and lithium responsivity in all study patients. The average interhemispheric coherence spectrum (i.e., containing QEEG multivariable composite Z-scores) of twenty-six responsive patients with affective and attentional disorders was analyzed. The spectra exhibited posterior delta hypocoherence (up to −1.7 mean-units), posterior theta hypocoherence (up to −1.4 mean-units), frontal alpha hypercoherence (up to +2.9 mean-units), and frontal beta hypercoherence (up to +1.7 mean units).

### EXAMPLE 5

### Nervous System Disorder Drug Response Probabilities Using Psychometric Testing Batteries

This example illustrates a variety of psychological test batteries and resulting exemplary scores that provide the probability of drug therapy responsiveness for a nervous system disorder.

Table IV will provide data showing the psychometric test Z scores predicting the probability of therapy success with a formulation comprising an anticonvulsant and a neuroactive modulator administered to a patient exhibiting at least one symptom of any nervous system disorder.

TABLE IV
Probability Response Categories Using Psychometric Test (PT) Battery Z Scores

| | SENSITIVE | | INTERMEDIATE | | | | RESISTIVE | |
|---|---|---|---|---|---|---|---|---|
| | Level 1 | Level 2 | Level 3 | Level 4 | Level 5 | Level 6 | Level 7 | Level 8 |
| PT | 100-90% | 90-80% | 80-65% | 65-50% | 50-35% | 35-20% | 20-10% | 10-0% |
| One | 2.00-1.75 | 1.75-1.50 | 1.50-1.25 | 1.25-1.00 | 1.00-0.75 | 0.75-0.50 | 0.50-0.25 | 0.25-0.10 |
| Two | 2.00-1.75 | 1.75-1.50 | 1.50-1.25 | 1.25-1.00 | 1.00-0.75 | 0.75-0.50 | 0.50-0.25 | 0.25-0.10 |
| Three | 2.00-1.75 | 1.75-1.50 | 1.50-1.25 | 1.25-1.00 | 1.00-0.75 | 0.75-0.50 | 0.50-0.25 | 0.25-0.10 |
| Four | 2.00-1.75 | 1.75-1.50 | 1.50-1.25 | 1.25-1.00 | 1.00-0.75 | 0.75-0.50 | 0.50-0.25 | 0.25-0.10 |
| Five | 2.00-1.75 | 1.75-1.50 | 1.50-1.25 | 1.25-1.00 | 1.00-0.75 | 0.75-0.50 | 0.50-0.25 | 0.25-0.10 |
| Six | 2.00-1.75 | 1.75-1.50 | 1.50-1.25 | 1.25-1.00 | 1.00-0.75 | 0.75-0.50 | 0.50-0.25 | 0.25-0.10 |
| Seven | 2.00-1.75 | 1.75-1.50 | 1.50-1.25 | 1.25-1.00 | 1.00-0.75 | 0.75-0.50 | 0.50-0.25 | 0.25-0.10 |
| Eight | 2.00-1.75 | 1.75-1.50 | 1.50-1.25 | 1.25-1.00 | 1.00-0.75 | 0.75-0.50 | 0.50-0.25 | 0.25-0.10 |
| Nine | 2.00-1.75 | 1.75-1.50 | 1.50-1.25 | 1.25-1.00 | 1.00-0.75 | 0.75-0.50 | 0.50-0.25 | 0.25-0.10 |

These data will demonstrate that patients exhibiting psychometric test battery Z scores between 0.10-0.50 have a low probability of a significant response to a formulation comprising an anticonvulsant and a neuroactive modulator. Patients exhibiting psychometric test battery Z scores between 0.50-1.50 have a likely probability of a significant response to a formulation comprising an anticonvulsant and a neuroactive modulator. Patients exhibiting psychometric test battery Z scores between 2.00-1.50 have a high probability of a significant response to a formulation comprising an anticonvulsant and a neuroactive modulator.

### EXAMPLE 6

### Nervous System Disorder Drug Response Probability Prediction Using Biological Indicators

This example will illustrate a variety of biological indicators and their exemplary scores that provide predictive indicators of drug therapy responsiveness for a nervous system disorder.

Table V will provide data showing the biological indicator Z scores predicting the probability of therapy success with a formulation comprising an anticonvulsant and a neuroactive modulator administered to a patient exhibiting at least one symptom of any nervous system disorder.

TABLE V
Probability Response Categories using Biological Indicator (BI) Z Scores

| | SENSITIVE | | INTERMEDIATE | | | | RESISTIVE | |
|---|---|---|---|---|---|---|---|---|
| | Level 1 | Level 2 | Level 3 | Level 4 | Level 5 | Level 6 | Level 7 | Level 8 |
| BI | 100-90% | 90-80% | 80-65% | 65-50% | 50-35% | 35-20% | 20-10% | 10-0% |
| One | 2.00-1.75 | 1.75-1.50 | 1.50-1.25 | 1.25-1.00 | 1.00-0.75 | 0.75-0.50 | 0.50-0.25 | 0.25-0.10 |
| Two | 2.00-1.75 | 1.75-1.50 | 1.50-1.25 | 1.25-1.00 | 1.00-0.75 | 0.75-0.50 | 0.50-0.25 | 0.25-0.10 |
| Three | 2.00-1.75 | 1.75-1.50 | 1.50-1.25 | 1.25-1.00 | 1.00-0.75 | 0.75-0.50 | 0.50-0.25 | 0.25-0.10 |
| Four | 2.00-1.75 | 1.75-1.50 | 1.50-1.25 | 1.25-1.00 | 1.00-0.75 | 0.75-0.50 | 0.50-0.25 | 0.25-0.10 |
| Five | 2.00-1.75 | 1.75-1.50 | 1.50-1.25 | 1.25-1.00 | 1.00-0.75 | 0.75-0.50 | 0.50-0.25 | 0.25-0.10 |
| Six | 2.00-1.75 | 1.75-1.50 | 1.50-1.25 | 1.25-1.00 | 1.00-0.75 | 0.75-0.50 | 0.50-0.25 | 0.25-0.10 |
| Seven | 2.00-1.75 | 1.75-1.50 | 1.50-1.25 | 1.25-1.00 | 1.00-0.75 | 0.75-0.50 | 0.50-0.25 | 0.25-0.10 |
| Eight | 2.00-1.75 | 1.75-1.50 | 1.50-1.25 | 1.25-1.00 | 1.00-0.75 | 0.75-0.50 | 0.50-0.25 | 0.25-0.10 |
| Nine | 2.00-1.75 | 1.75-1.50 | 1.50-1.25 | 1.25-1.00 | 1.00-0.75 | 0.75-0.50 | 0.50-0.25 | 0.25-0.10 |

These data will demonstrate that patients exhibiting biological indicator Z scores between 0.10-0.50 have a low probability of a significant response to a formulation comprising an anticonvulsant and a neuroactive modulator. Patients exhibiting biological indicator Z scores between 0.50-1.50 have a likely probability of a significant response to a formulation comprising an anticonvulsant and a neuroactive modulator. Patients exhibiting biological indicator Z scores between 2.00-1.50 have a high probability of a significant response to a formulation comprising an anticonvulsant and a neuroactive modulator.

### EXAMPLE 7

### Nervous System Disorder Drug Response Probability Prediction Using Brain Cognitive Indicators

This example will illustrate a variety of brain metabolic indicators and their exemplary scores that provide predictive indicators of drug therapy responsiveness for a nervous system disorder.

Table VI will provide data showing the brain cognitive indicator Z scores predicting the probability of therapy success with a formulation comprising an anticonvulsant and a neuroactive modulator administered to a patient exhibiting at least one symptom of any nervous system disorder.

TABLE VI
Probability Categories Using Brain Cognitive Indicator (BCI) Z Scores

| | SENSITIVE | | INTERMEDIATE | | | | RESISTIVE | |
|---|---|---|---|---|---|---|---|---|
| | Level 1 | Level 2 | Level 3 | Level 4 | Level 5 | Level 6 | Level 7 | Level 8 |
| BCI | 100-90% | 90-80% | 80-65% | 65-50% | 50-35% | 35-20% | 20-10% | 10-0% |
| One | 2.00-1.75 | 1.75-1.50 | 1.50-1.25 | 1.25-1.00 | 1.00-0.75 | 0.75-0.50 | 0.50-0.25 | 0.25-0.10 |
| Two | 2.00-1.75 | 1.75-1.50 | 1.50-1.25 | 1.25-1.00 | 1.00-0.75 | 0.75-0.50 | 0.50-0.25 | 0.25-0.10 |
| Three | 2.00-1.75 | 1.75-1.50 | 1.50-1.25 | 1.25-1.00 | 1.00-0.75 | 0.75-0.50 | 0.50-0.25 | 0.25-0.10 |
| Four | 2.00-1.75 | 1.75-1.50 | 1.50-1.25 | 1.25-1.00 | 1.00-0.75 | 0.75-0.50 | 0.50-0.25 | 0.25-0.10 |
| Five | 2.00-1.75 | 1.75-1.50 | 1.50-1.25 | 1.25-1.00 | 1.00-0.75 | 0.75-0.50 | 0.50-0.25 | 0.25-0.10 |
| Six | 2.00-1.75 | 1.75-1.50 | 1.50-1.25 | 1.25-1.00 | 1.00-0.75 | 0.75-0.50 | 0.50-0.25 | 0.25-0.10 |
| Seven | 2.00-1.75 | 1.75-1.50 | 1.50-1.25 | 1.25-1.00 | 1.00-0.75 | 0.75-0.50 | 0.50-0.25 | 0.25-0.10 |
| Eight | 2.00-1.75 | 1.75-1.50 | 1.50-1.25 | 1.25-1.00 | 1.00-0.75 | 0.75-0.50 | 0.50-0.25 | 0.25-0.10 |
| Nine | 2.00-1.75 | 1.75-1.50 | 1.50-1.25 | 1.25-1.00 | 1.00-0.75 | 0.75-0.50 | 0.50-0.25 | 0.25-0.10 |

These data will demonstrate that patients exhibiting brain cognitive indicator Z scores between 0.10-0.50 have a low probability of a significant response to a formulation comprising an anticonvulsant and a neuroactive modulator. Patients exhibiting brain cognitive indicator Z scores between 0.50-1.50 have a likely probability of a significant response to a formulation comprising an anticonvulsant and a neuroactive modulator. Patients exhibiting brain cognitive indicator Z scores between 2.00-1.50 have a high probability of a significant response to a formulation comprising an anticonvulsant and a neuroactive modulator.

### EXAMPLE 8

### Nervous System Disorder Drug Response Probability Prediction Using Genotype Profiling

This example will illustrate a variety of genotype profiles and their exemplary scores that provide predictive indicators of drug therapy responsiveness for a nervous system disorder.

Table VII will provide data showing the genotype profile Z scores predicting the probability of therapy success with a formulation comprising an anticonvulsant and a neuroactive modulator administered to a patient exhibiting at least one symptom of any nervous system disorder.

TABLE VII
Probability Categories Using Genotype Allelic Profile (GAP) Scores

| | SENSITIVE | | INTERMEDIATE | | | | RESISTIVE | |
|---|---|---|---|---|---|---|---|---|
| | Level 1 | Level 2 | Level 3 | Level 4 | Level 5 | Level 6 | Level 7 | Level 8 |
| GAP | 100-90% | 90-80% | 80-65% | 65-50% | 50-35% | 35-20% | 20-10% | 10-0% |
| One | 2.00-1.75 | 1.75-1.50 | 1.50-1.25 | 1.25-1.00 | 1.00-0.75 | 0.75-0.50 | 0.50-0.25 | 0.25-0.10 |
| Two | 2.00-1.75 | 1.75-1.50 | 1.50-1.25 | 1.25-1.00 | 1.00-0.75 | 0.75-0.50 | 0.50-0.25 | 0.25-0.10 |
| Three | 2.00-1.75 | 1.75-1.50 | 1.50-1.25 | 1.25-1.00 | 1.00-0.75 | 0.75-0.50 | 0.50-0.25 | 0.25-0.10 |
| Four | 2.00-1.75 | 1.75-1.50 | 1.50-1.25 | 1.25-1.00 | 1.00-0.75 | 0.75-0.50 | 0.50-0.25 | 0.25-0.10 |
| Five | 2.00-1.75 | 1.75-1.50 | 1.50-1.25 | 1.25-1.00 | 1.00-0.75 | 0.75-0.50 | 0.50-0.25 | 0.25-0.10 |
| Six | 2.00-1.75 | 1.75-1.50 | 1.50-1.25 | 1.25-1.00 | 1.00-0.75 | 0.75-0.50 | 0.50-0.25 | 0.25-0.10 |
| Seven | 2.00-1.75 | 1.75-1.50 | 1.50-1.25 | 1.25-1.00 | 1.00-0.75 | 0.75-0.50 | 0.50-0.25 | 0.25-0.10 |
| Eight | 2.00-1.75 | 1.75-1.50 | 1.50-1.25 | 1.25-1.00 | 1.00-0.75 | 0.75-0.50 | 0.50-0.25 | 0.25-0.10 |
| Nine | 2.00-1.75 | 1.75-1.50 | 1.50-1.25 | 1.25-1.00 | 1.00-0.75 | 0.75-0.50 | 0.50-0.25 | 0.25-0.10 |

These data will demonstrate that patients exhibiting genotype allelic profile Z scores between 0.10-0.50 have a low probability of a significant response to a formulation comprising of an anticonvulsant and a neuroactive modulator. Patients exhibiting genotype allelic profile Z scores between 0.50-1.50 have a likely probability of a significant response to a formulation comprising an anticonvulsant and a neuroactive modulator. Patients exhibiting genotype allelic profile Z scores between 2.00-1.50 have a high probability of a significant response to a formulation comprising of an anticonvulsant and a neuroactive modulator.

### EXAMPLE 9

### Retrospective QEEG Analysis

This example presents data from a retrospective study validating the QEEG prognosis prediction protocol.

This study included fifty-four (54) patients with clinical depression and 46 patients with attentional disorders. Medication-free EEG recordings were taken on each of the patients by making certain that they received no drugs for at least seven (7) half-lives. After the EEGs for each patient were recorded, each patient received “conventional” DSM-directed treatment (i.e., depressed patients were first treated with antidepressants and attentionally disrdered patients were first treated with stimulants). At the end of twenty-six (26) weeks of antidepressant therapy a CGI score was determined for each patient.

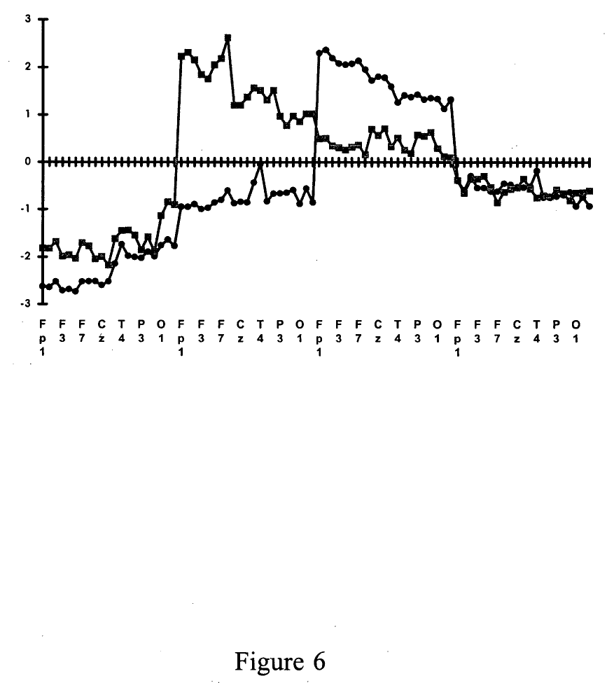

The QEEG patterns of the fifty-four (54) patients with clinical depression are shown in FIG. 6 where approximately 86% responded favorable to treatment. The majority of depressed patients (i.e., 35) had excess frontal alpha wave patterns with diminished delta and theta wave patterns. This picture is similar to the standard QEEG pattern found in a convalescent database for patients responding favorable to antidepressant treatment.

The remainder of the patients (i.e., 7) had excess theta wave patterns, normal alpha wave and low delta wave patterns. Of these patients, only 29% responded to antidepressant therapy. Using the present invention, the model algorithm generated for this 29% would have detected the shift in affected band frequencies and predicted that the patients would have responded to stimulant therapy.

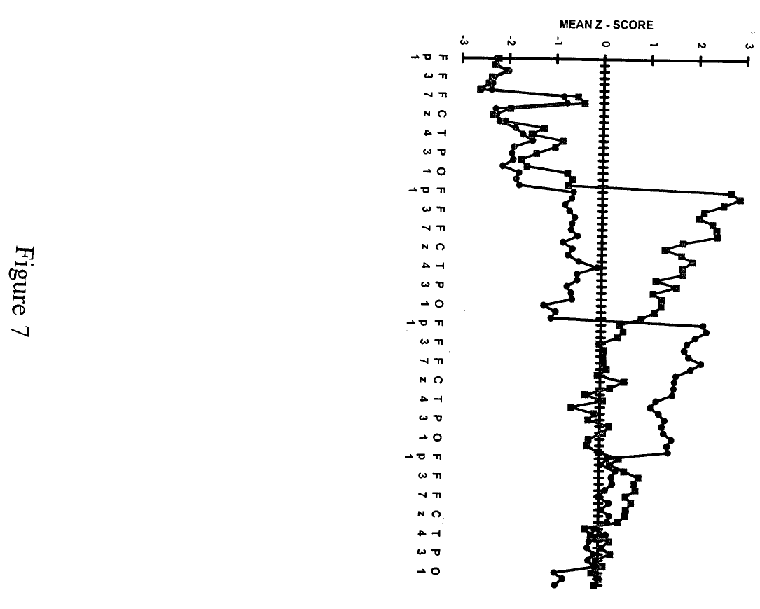

In the forty-six (46) patients with attentional disorders, QEEG patterns determined that a minority of the patients (14) had excess theta wave patterns, with normal alpha wave and decreased delta wave patterns. See FIG. 7. This pattern is similar to the QEEG pattern of stimulant responders, and in fact 100% of these patients responded favorably to stimulant therapy.

Interestingly, the majority of the patients (25) with attentional disorders had excess alpha wave and normal theta wave patterns. As discussed above, this is similar to the QEEG pattern that predicts a favorably antidepressant outcome. Consequently, this group of patients were non-remissive when given the DSM-directed therapy of stimulants. However, 87% of the patients responded favorably when given antidepressants after failing to respond to stimulants (note that this is a counter-intuitive treatment for ADD). If these patients had been given a QEEG screening prior to drug therapy, antidepressants would have been immediately prescribed.

These retrospective studies revealed very clear heterogeneities in EEG patterns of patients having either depression or attentional disorders. QEEG revealed those depressed patients that should respond favorably to the conventional DSM-directed pharmacotherapy of this disorder, but more importantly, identified those that are not likely to respond to the conventional treatment. In the case of the patients with attentional disorders, the QEEG analysis correctly identified the patients that did, and did not, respond to conventional therapy. In fact, QEEG predicted the effective therapies over 87% of the time, a far greater percentage than found with standard clinician drug selection procedures. In conclusion, this retrospective study revealed clearly that there are markers within the QEEG that are better indicators of medication responsivity than the conventional DSM-directed treatment regimens.

### EXAMPLE 9

### Prospective QEEG Analysis

This example presents data from a prospective study validating the QEEG prognosis prediction protocol.

Medication-free EEGs were obtained on thirteen (13) depressed patients unresponsive to medication treatment for an average of two (2) years. The patients, blinded to treatment modality, were divided into a control group, in which conventional DSM-directed antidepressant pharmacotherapy was administered and an experimental group in which antidepressant pharmacotherapy was determined by QEEG analysis preselection according to the present invention. The clinical outcomes were assessed using CGI scoring.

In the group of patients that were treated with DSM-directed antidepressant pharmacotherapy 17% (i.e., 1 out of 6) demonstrated a marked improvement (i.e., CGI=3). In the QEEG-directed antidepressant pharmacotherapy group, 86% (i.e., 6 out of 7) demonstrated significant or marked improvement (i.e., CGI of 2 or 3, respectively). The single responding patient in the conventual pharmacotherapy group demonstrated a QEEG pattern predicting a favorably response.

Clearly, a QEEG analysis is highly useful in predetermining which patients exhibiting at least one symptom of depression will respond to an antidepressant therapy.

1. A formulation comprising an adrenergic beta-blocker and a stimulant.

2. The formulation of claim 1, wherein said beta-blocker comprises propranolol and said stimulant comprises dextroamphetamine.

3. The formulation of claim 1, wherein said beta-blocker comprises atenolol and said stimulant comprises Tryptophan-Phenylalaine-Glutamine.

4. The formulation of claim 1, wherein said stimulant is selected from the group consisting of amphetamine, dextroamphetamine, methamphetamine, modafinil, methylphenidate, atomoxetine, ephedrine, amantadine, caffeine, theophylline, theobromine, Tryptophan-Phenylalanine-Glutamine and ginko biloba.

5. The formulation of claim 1, wherein said adrenergic beta-blocker is selected from the group consisting of prenalteraol, xamoterol, propranolol, atenolol, betaxolol, nadolol, carvedilol, sotolol, timolol, labetolol, acebutolol, pindolol, esmolol, metoprolol, bisoporol and bucindolol.

6. The formulation of claim 1, wherein the form of said formulation is selected from the group consisting of a tablet, capsule, oral liquid, intrapulmonary liquid, transdermal patch, a polymer-coated tablet, a microparticle, a nanoparticle, an aerosol, fast-dissolve compound and a sterile injectable solution.

7. A method of treatment, comprising:
a) providing a patient exhibiting at least one symptom of a nervous system disorder, and b) administering to the patient a formulation comprising an adrenergic beta-blocker and a stimulant such that at least one symptom of said nervous system disorder is reduced.

8. The method of claim 7, wherein said nervous system disorder is selected from the group consisting of childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders.

9. The formulation of claim 7, wherein said stimulant is selected from the group consisting of amphetamine, dextroamphetamine, methamphetamine, modafinil, methylphenidate, atomoxetine, ephedrine, caffeine, theophylline, theobromine, Tryptophan-Phenylalanine-Glutamine and ginko biloba.

10. The method of claim 7, wherein said formulation comprises a compounded formulation.

11. A method of treatment, comprising:
a) providing a patient exhibiting at least one symptom of a nervous system disorder and is being treated with a dose of a third drug, wherein said patient is non-remissive; and
b) administering to said patient a formulation comprising a dose of an adrenergic beta-blocker and a dose of a stimulant such that at least one symptom of said nervous system disorder is reduced.

12. The method of claim 11, wherein said formulation further comprises said third drug.

13. The method of claim 11, further comprising step (c), decreasing said dose of said third drug.

14. The method of claim 11, wherein said admistering of step (b) is performed over a period of time such that said dose of said blocker and said stimulant is increased.

15. The method of claim 11, wherein said nervous system disorder is selected from the group comprising childhood disorders, cognitive disorders, substance disorders, schizophrenia, psychotic disorders mood disorders, anxiety disorders, somatoform disorders, factitious disorders, dissociative disorders, sexual disorders, gender identity disorders, eating disorders, sleep disorders, impulse-control disorders, adjustment disorders or personality disorders.

16. The method of claim 11, wherein said drug is selected from the group comprising selective serotonin reuptake inhibitors, monoamine oxidase inhibitors, antipsychotic drugs, antianxiety/anixolytic drugs, barbiturates, stimulants, antiparkinsonian drugs, analgesic drugs, cardiac agents and nutriccuticals.

17. The method of claim 11, wherein said formulation comprises a compounded formulation.

18. The method of claim 17, wherein said compounded formulation comprises said third drug.

19. A formulation comprising a adrenergic beta blocker and a monoamine oxidase inhibitor, wherein said monoamine oxidase inhibitor is selected from the group consisting of selegiline and moclobemide.

20. The formulation of claim 19, wherein said adrenergic beta blocker is selected from the group consisting of prenalteraol, xamoterol, propranolol, atenolol, betaxolol, nadolol, carvedilol, sotolol, timolol, labetolol, acebutolol, pindolol, esmolol, metoprolol, bisoporol and bucindolol
